(19) United States
(12) Patent Application Publication
(10) Pub. No.: US 2021/0322511 A1
(43) Pub. Date: Oct. 21, 2021

(54) METHODS AND COMPOSITIONS FOR TREATING A RESPIRATORY DISEASE

(71) Applicant: PRESIDENT AND FELLOWS OF HARVARD COLLEGE, Cambridge, MA (US)

(72) Inventors: Tom A. RAPOPORT, Brookline, MA (US); Nicholas O. BODNAR, Cambridge, MA (US); Goran MILICIC, Cambridge, MA (US); Navdar SEVER, Cambridge, MA (US)

(73) Assignee: PRESIDENT AND FELLOWS OF HARVARD COLLEGE, Cambridge, MA (US)

(21) Appl. No.: 17/232,891

(22) Filed: Apr. 16, 2021

Related U.S. Application Data
(60) Provisional application No. 63/011,521, filed on Apr. 17, 2020.

(51) Int. Cl. A61K 38/16; C07K 14/47; A61K 47/24

(52) U.S. Cl. CPC A61K 38/16; C07K 2319/50; A61K 47/24; C07K 14/47

(57) ABSTRACT

Provided herein are methods and compositions related to treating a respiratory disease and a method of making a lung surfactant composition.

### CROSS-REFERENCE TO RELATED APPLICATIONS

This application claims benefit under 35 U.S.C. § 119(e) of U.S. Provisional Application No. 63/011,521 filed on Apr. 17, 2020, the contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 15, 2021, is named 002806-093130USPT_SL.txt and is 63,341 bytes in size.

### TECHNICAL FIELD

The technology described herein relates to methods of making a lung surfactant, treating a respiratory disease, and uses thereof.

### BACKGROUND

Respiration is a key component of human life. The lungs remove oxygen from air for transport via the blood stream to the entire body. surfactant B protein (SP-B) is a surfactant essential for breathing in mice and humans. It is made in alveolar type II cells as a precursor containing three domains related to the lysosome-localized saposins. The precursor is proteolytically processed into the individual domains, with the middle domain (SP-BM) currently considered to be the biologically important “mature” protein. SP-BM is initially stored in lamellar bodies (LB), lysosome-like organelles, in which membrane sheets are densely stacked on top of each other. LBs are eventually exocytosed to generate extracellular surfactant, a mixture of phospholipids and protein. While surfactants derived from animals are available commercially, the surfactant mixture has proven to be ineffective in adults that suffer from respiratory diseases. Furthermore, generating SP-BM recombinantly has been a challenge in the industry and improved methods of manufacturing surfactants are needed.

### SUMMARY

The methods and compositions provided herein are based in part, on the discovery that the domains of surfactant B protein (SP-B) each function in lamellar body (LB) formation and that the SP-BM domain alone can be expressed and purified to generate LB-like structures.

In one aspect, provided herein is a lung surfactant composition comprising:

a. a polypeptide comprising a surfactant B protein N-terminal domain (SP-BN); and

b. at least one phospholipid

In another aspect, provided herein is a lung surfactant composition comprising:

a. a polypeptide comprising a surfactant B protein middle domain (SP-BM); and

b. at least one phospholipid

In another aspect, provided herein is a method of treating a respiratory disease in a subject, the method comprising: administering to a subject in need thereof a lung surfactant composition provided herein.

In another aspect, provided herein is a method of treating a respiratory disease in a subject, the method comprises: administering through the trachea a composition or a vector encoding a polypeptide provided herein to a subject in need thereof.

In another aspect, provided herein is a vector comprising a nucleic acid sequence encoding SP-BM; SP-BN; or SP-BM and SP-BN.

In one embodiment of any of the aspects, the polypeptide provided herein further comprises a surfactant B protein N-terminal domain (SP-BN), and/or a surfactant B protein middle domain (SP-BM), and/or a surfactant B protein C-terminal domain (SP-BC), or any combination thereof. In another embodiment of any of the aspects, the polypeptide is a human, bovine, or mouse polypeptide. In another embodiment of any of the aspects, the SP-BN is human, bovine, or mouse SP-BN. In another embodiment of any of the aspects, the SP-BM is human, bovine, or mouse SP-BM.

In another embodiment of any of the aspects, the SP-BM comprises a cysteine (C, Cys) to alanine (A, Ala) amino acid substitution at position 48 (C48A). In another embodiment of any of the aspects, the SP-BN comprises a lysine to glutamic acid to glutamine (K46E) amino acid substitution at position 46, a proline to cysteine (P50C) amino acid substitution at position 50, an arginine to glutamine (R51E) amino acid substitution at position 51, a tyrosine to alanine (Y59A) amino acid substitution at position 59, and/or a histidine to alanine (H79A) amino acid substitution, or any combination thereof.

In another embodiment of any of the aspects, the phospholipid is a glycerophospholipid. In another embodiment of any of the aspects, the phospholipid is selected from the group consisting of: 1,2-dipalmitoyl-sn-glycero-3-phosphocholine (DPPC); phosphatidylglycerol (PG); phosphatidylcholine (PC), phosphatidylethanolamine (PE), phosphatidylserine (PS), and phosphatidylinositol (PI). In another embodiment of any of the aspects, the lung surfactant composition further comprises DPPC, PG, or a combination thereof.

In another embodiment of any of the aspects, the composition is formulated with a pharmaceutically acceptable carrier. In another embodiment of any of the aspects, the lung surfactant composition is formulated for intratracheal delivery.

In another embodiment of any of the aspects, the lung surfactant composition further comprises a Surfactant Protein C (SP-C) or a functional fragment thereof. In another embodiment of any of the aspects, the lung surfactant composition further comprises an additional lung surfactant.

In another embodiment of any of the aspects, the subject is a mammal. In another embodiment of any of the aspects, the subject is a human. In another embodiment of any of the aspects, the subject has or is suspected of having a respiratory disease.

In another embodiment of any of the aspects, the respiratory disease is selected from the group consisting of: acute respiratory distress syndrome (ARDS), neonatal respiratory distress syndrome (NRDS), pneumonia, asthma, meconium aspiration syndrome, respiratory failure, chronic obstructive pulmonary disease (COPD), and a lung infection.

In another aspect, provided herein is a method for a preparing a polypeptide comprising a surfactant B protein middle domain (SP-BM), the method comprising:

a. expressing a fusion protein in a cell, wherein the fusion protein comprises:
i. a peptide tag;
ii. a surfactant B protein N-terminal domain (SP-BN);
iii. a linker; and
iv. a SP-BM domain; and

b. cleaving the linker in the expressed protein; and

c. isolating or purifying the polypeptide comprising the SP-BM domain.

In one embodiment of any of the aspects, the linker comprises a cleavage site. In another embodiment of any of the aspects, the cleavage site is a protease cleavage site or a chemical cleavage site. In another embodiment of any of the aspects, the linker is a thrombin cleavage linker. In another embodiment of any of the aspects, the cleavage site is selected from the group consisting of: a tobacco mosaic virus (TEV) protease cleavage site, a thrombin cleavage site, a factor Xa cleavage site, a 3C protease site, and a specific nickel-assisted cleavage (SNAC)-tag.

In another embodiment of any of the aspects, the isolating or purification in step (c) comprises chromatography. In another embodiment of any of the aspects, the isolating or purification in step (c) comprises size exclusion chromatography (SEC).

In another embodiment of any of the aspects, the method further comprises solubilizing the expressed protein prior to the cleaving step. In another embodiment of any of the aspects, the solubilizing comprises solubilizing the expressed protein in a detergent. In another embodiment of any of the aspects, the detergent is sarkosyl. In another embodiment of any of the aspects, the solubilizing is in octylglucoside.

In another embodiment of any of the aspects, the method further comprises a step of preisolating/prepurifying the cleaved polypeptide after the cleaving step. In another embodiment of any of the aspects, the preisolating/prepurifying step comprises contacting the cleaved polypeptide with a hydrophobic resin. In another embodiment of any of the aspects, the method further comprises solubilizing the cleaved polypeptide after the cleaving step and prior to the isolating/purifying step.

In another embodiment of any of the aspects, the method further comprises extracting the cleaved polypeptide after the cleaving step. In another embodiment of any of the aspects, the extraction is in chloroform/methanol.

In another embodiment of any of the aspects, the cell is a bacterial cell, a yeast cell, an insect cell, an amphibian cell, or a mammalian cell. In another embodiment of any of the aspects, the cell is an Escherichia coli cell.

In another embodiment of any of the aspects, the fusion protein lacks a surfactant B protein C-terminal domain (SP-BC). In another embodiment of any of the aspects, the fusion protein further comprises a saposin polypeptide between the peptide tag and the SP-BN domain. In another embodiment of any of the aspects, the saposin polypeptide comprises a saposin A domain (SapA). In another embodiment of any of the aspects, the fusion protein comprises an amino acid sequence of SEQ ID NO: 4 or SEQ ID NO: 16.

In another embodiment of any of the aspects, the peptide tag is selected from the group consisting of: a glutathione S-transferase (GST) tag, a FLAG-tag, a Myc-tag, HALO, a maltose-binding peptide, and a hexa-his peptide (6×His).

In another aspect, provided herein is a polypeptide prepared by the methods provided herein.

In another aspect, provided herein is a fusion protein comprising:

i. a peptide tag;

ii. a surfactant B protein N-terminal domain (SP-BN);

iii. a linker; and

iv. a surfactant B protein middle domain (SP-BM) domain.

In another aspect, provided herein is a nucleic acid encoding the fusion protein.

### Definitions

For convenience, the meanings of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in cellular and molecular biology, and biochemistry can be found in The Merck Manual of Diagnosis and Therapy, 20th Edition, published by Merck Sharp & Dohme Corp., 2018 (ISBN 9780911910421, 0911910425); Robert S. Porter et al. (eds.), The Encyclopedia of Molecular Cell Biology and Molecular Medicine, published by Blackwell Science Ltd., 2008 (ISBN 3527305424, 9783527305421); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2016 (ISBN 9780815345510, 0815345518); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Laboratory Methods in Enzymology: RNA, Jon Lorsch (ed.) Elsevier, 2013 (ISBN: 9780124200371, 0124200370); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), Immunological Methods, Ivan Lefkovits, Benvenuto Pernis, (eds.) Elsevier Science, 2014 (ISBN: 9781483269993, 148326999X), the contents of which are all incorporated by reference herein in their entireties.

As used herein, the terms “treat,” “treatment,” “treating,” or “amelioration” refers to therapeutic treatments, wherein the object is to reverse, alleviate, ameliorate, inhibit, slow down or stop the progression or severity of a condition associated with a respiratory disease, e.g. adult respiratory distress syndrome (ARDS). The term “treating” includes reducing or alleviating at least one adverse effect or symptom of respiratory disease, for example, difficulty breathing. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the terms “administering,” and “injecting” are used interchangeably in the context of the placement of an agent (e.g., a surfactant described herein), into a subject, by a method or route which results in at least partial localization of the agent at a desired site, such as the trachea or a region thereof, such that a desired effect(s) is produced (e.g., increase in lung surfactant level or activity). The agent described herein can be administered by any appropriate route which results in delivery to a desired location in the subject. The half-life of the agent after administration to a subject can be as short as a few minutes, hours, or days, e.g., twenty-four hours, to a few days, to as long as several years, i.e., long-term. In some embodiments of any of the aspects, the term “administering” refers to the administration of a lung surfactant composition provided herein. The administering can be done by local application, direct injection (e.g., directly administered to the lungs), a nebulizer, or nasal delivery to the subject in need thereof. Administering can be local or systemic.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a respiratory disease, or has never received treatment for a respiratory disease. A subject can have previously been diagnosed with having a respiratory disease, or has never been diagnosed with a respiratory disease.

Preferably, the subject is a mammal. The mammal can be a human, non-human primate, mouse, rat, dog, cat, horse, or cow, but are not limited to these examples. Mammals other than humans can be advantageously used as subjects that represent animal models of a respiratory disease (e.g. lung injury or ARDS). In addition, the methods described herein can be used to treat domesticated animals and/or pets. A subject can be male or female. A subject can be one who has been previously diagnosed with or identified as suffering from a respiratory disease, but need not have already undergone treatment.

The term “healthy subject” as used herein refers to a subject that, at a minimum, lacks markers or symptoms of the disease or disorder to be treated (e.g., a respiratory disease).

As used herein, the term “polypeptide” is intended to encompass a singular “polypeptide” as well as plural “polypeptides,” and includes any chain or chains of two or more amino acids. Thus, as used herein, terms including, but not limited to “peptide,” “dipeptide,” “tripeptide,” “protein,” “enzyme,” “amino acid chain,” and “contiguous amino acid sequence” are all encompassed within the definition of a “polypeptide,” and the term “polypeptide” can be used instead of, or interchangeably with, any of these terms. The term further includes polypeptides that have undergone one or more post-translational modification(s), including for example, but not limited to, glycosylation, acetylation, phosphorylation, amidation, derivatization, proteolytic cleavage, post-translation processing, or modification by inclusion of one or more non-naturally occurring amino acids. Conventional nomenclature exists in the art for polynucleotide and polypeptide structures. For example, one-letter and three-letter abbreviations are widely employed to describe amino acids: Alanine (A; Ala), Arginine (R; Arg), Asparagine (N; Asn), Aspartic Acid (D; Asp), Cysteine (C; Cys), Glutamine (Q; Gln), Glutamic Acid (E; Glu), Glycine (G; Gly), Histidine (H; His), Isoleucine (I; Ile), Leucine (L; Leu), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val), and Lysine (K; Lys). Amino acid residues provided herein are preferred to be in the “L” isomeric form. However, residues in the “D” isomeric form may be substituted for any L-amino acid residue provided the desired properties of the polypeptide are retained.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. For example, increasing activity can refer to activating surfactant production or increasing levels of surfactant B protein directly or indirectly.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a respiratory disease, or a biological sample that has not been contacted with a composition disclosed herein).

As used herein, an “appropriate control” refers to an untreated, otherwise identical cell or population (e.g., a biological sample that was not contacted by a composition described herein, or not contacted in the same manner, e.g., for a different duration, as compared to a non-control cell).

The term “pharmaceutically acceptable” can refer to compounds and compositions which can be administered to a subject (e.g., a mammal or a human) without undue toxicity.

As used herein, the term “pharmaceutical composition” can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture media. Non-limiting examples of pharmaceutical carriers include particle or polymer-based vehicles such as nanoparticles, microparticles, polymer microspheres, or polymer-drug conjugates. For example, the pharmaceutical compositions provided herein comprise a surfactant B protein domain provided herein (e.g., SP-BM, SP-BN, or a combination thereof) and a pharmaceutically acceptable carrier for administration of the surfactant to the lungs.

As used herein the term “respiratory disease” refers to any disease that affects the respiratory system, lungs, or a subject's ability to breathe. The respiratory disease can cause at least one symptom of the disease. These symptoms can include but are not limited to, difficulty breathing, too much or too little of surfactant in the lungs, wheezing, tightness in the chest, or any other symptom associated with a respiratory disease in a subject. Non-limiting examples of respiratory diseases include acute respiratory distress syndrome (ARDS), neonatal respiratory distress syndrome (NRDS), pneumonia, asthma, meconium aspiration syndrome, respiratory failure, chronic obstructive pulmonary disease (COPD), and lung infections.

As used herein, the terms “lung surfactant” or “lung surfactant composition” or “pulmonary surfactant” or “composition” are used interchangeably to refer to a composition comprising of a polypeptide provided herein and at least one phospholipid. A lung surfactant can be a synthetic lung surfactant made by the methods provided herein. Alternatively, the lung surfactant can be naturally occurring, e.g., as made by type II alveolar cells, or can isolated and purified from a lung (e.g., bovine lung) by methods known in the art. At a minimum, the lung surfactant compositions provided herein will comprise at least one N-terminal domain of SP-B (SP-BN), and/or at least one middle domain of SP-B (SP-BM), a combination thereof; and at least one phospholipid (e.g., a glycerophospholipid). The SP-B polypeptide domains provided herein can be isolated or derived from any organism, e.g., a mammal.

As used herein, the term “surfactant B protein” or “SP-B” refers to a lipid-associated protein found in pulmonary surfactant. SP-B is a surfactant protein essential for breathing. Sequences for SP-B are known in the art. For example, the nucleic acid NCBI reference sequences for human surfactant protein B (encoded by SFTPB) is Gene ID: 6439 and for mouse SP-B is Gene ID: ID: 20388. The human polypeptide sequence of SP-B is provided herein as SEQ ID NO: 1. The term “SP-B” can refer to the full length precursor polypeptide comprising all the domains (e.g., SP-BN, SP-BM, and SP-BC) or can refer to the proteolytically processed SP-B.

As used herein, the term “surfactant B protein N-terminal domain” or “SP-BN” or “N-terminal saposin-like domain” refer to the N-terminal region of the full length SP-B protein. SP—BN is a polypeptide that generally contains about 86 amino acids, including 6 cysteines that form 3 disulfide bridges. The human polypeptide sequence of SP-BN is provided herein as SEQ ID NO: 2.

As used herein, the term “surfactant B protein middle domain” or “SP-BM” or “middle saposin-like domain” refers to the middle region of the full length SP-B protein. SP-BM is a polypeptide that generally contains about 79 amino acids that is considered to be the mature protein important for LB formation and surfactant function. The human polypeptide sequence of SP-BM is provided herein as SEQ ID NO: 3.

As used herein, the term “surfactant B protein C-terminal domain” or “SP-BC” refers to the C-terminal region of the full length SP-B protein. The human polypeptide sequence of SP-BC is provided herein as SEQ ID NO: 6.

As used herein, the term “surfactant protein C” or “SP-C” or “SP-BC” refers to another pulmonary surfactant protein. Generally, SP-C is made as a single-spanning membrane protein with a short cytosolic N-terminus and a luminal C-terminal segment containing a folded BRICHOS domain. Humans and animals born lacking SP-C can develop progressive interstitial lung disease. The human polypeptide sequences of SP-C and SP-C isoforms are provided herein as SEQ ID NO: 13 and SEQ ID NO: 14.

As used herein, the term “fusion protein” refers to an engineered polypeptide or protein that is linked to another polypeptide. For example, the term can refer to, but is not limited to, a fusion of at least one SP-BN polypeptide with at least one SP-BM polypeptide domains and substantially lacks an SP-B C terminal domain (SP-BC).

As used herein, a “functional fragment” is a fragment or segment of a peptide which retains at least 50% of the wild type reference polypeptide's activity according to an assay known in the art or described below herein. A functional fragment can comprise conservative substitutions of the sequences disclosed herein. For example, the functional fragment of the polypeptides provided herein can permit the formation of lamellar bodies or increase respiratory function in a subject.

As used herein, the term “linker” means a molecular moiety that connects two parts of a composition. The linker can be, for example, a polypeptide linker or a nucleic acid linker.

The term “lipid” is used in the conventional sense and includes compounds of varying chain length, from as short as about 2 carbon atoms to as long as about 28 carbon atoms. Additionally, the compounds may be saturated or unsaturated and in the form of straight- or branched-chains or in the form of unfused or fused ring structures. Exemplary lipids include, but are not limited to, fats, waxes, sterols, steroids, bile acids, fat-soluble vitamins (such as A, D, E, and K), monoglycerides, diglycerides, phospholipids, glycolipids, sulpholipids, aminolipids, chromolipids (lipochromes), glycerophospholipids, sphingolipids, prenol lipids, saccharolipids, polyketides, and fatty acids.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., SP-B, SP-BN, and/or SP-BM) from nucleic acid sequences contained therein linked to transcriptional regulatory sequences on the vector.

As used herein, the term “viral vector” refers to a nucleic acid vector construct that includes at least one element of viral origin and has the capacity to be packaged into a viral vector particle.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

### BRIEF DESCRIPTION OF THE DRAWINGS

This patent or application file contains at least one drawing executed in color. Copies of this patent or patent application publication with color drawing (s) will be provided by the Office upon request and payment of the necessary fee.

FIGS. 1A-1D show EM images of lamellar bodies (LBs) and tubular myelin. (FIG. 1A) Thin-section EM of a LB⁸⁷. (FIG. 1B) Tubular myelin. The inset shows a magnification of the regular membrane array. The arrow points to an apparent 4-way junction⁸⁷. (FIG. 1C) A human LB with projection core (PC). MC indicates the matrix core. The inset shows the PC with a hexagonal arrangement of protein molecules⁵⁶. (FIG. 1D) Immuno-EM staining for SP-BM. Staining is mostly seen in PCs⁵⁷.

FIGS. 2A-2B show sequence comparison of surfactant protein B (SP-B) domains and saposins. (FIG. 2A) Domain structures of SP-B and the related saposins. (FIG. 2B) Sequence alignment of SP-B domains and saposins. Note that all proteins have 6 conserved cysteines (shown in green).

FIG. 3 shows predicted localization of SP-BM at sheet edges. SP-BM is shown in red. The localization of SP-BM would be similar to that of detergent molecules in bicelles (right).

FIG. 4 shows a model for the generation of lamellar bodies (LBs). LBs are assumed to originate from multi-vesicular bodies (MVBs), which contain small vesicles. Both SP-BN and SP-BM are assumed to be transported into MVBs from other precursor organelles by vesicular transport. The ABC transporter ABCA3 moves phospholipid (PL) molecules from the outer to the inner leaflet of the limiting membrane, from where they are transferred by SP-BN to the vesicles along the concentration gradient. The vesicles grow into sheets that contain SP-BM at the edges. The sheets bend and SP-BM self-associates, forming the projection core (PC).

FIG. 5. Gel filtration of SP-BN. The peak fractions were subjected to SDS-PAGE and Coomassie blue staining. The band at the bottom is lipid.

FIGS. 6A-6C show the crystal structure of SP-BN. (FIG. 6A) The two monomers in the dimer are shown in red and grey, the two lateral PL molecules in green in stick presentation, and the central PL in grey. The “roof” is facing the viewer. (FIG. 6B) View from the side, with the “floor” on the bottom and the “roof” on the top. (FIG. 6C) View of PL molecules only. The two ends of the groove are left and right. Note that the hydrocarbon chains of the green and blue lateral PL molecules are curved, whereas the central PL has chains that extend throughout the groove.

FIG. 7 shows lipid transfer by SP-BN shown with a fluorescence dequenching assay. Liposomes containing two fluorescent PLs at quenching concentrations are incubated with SP-BN with or without unlabeled acceptor vesicles. The curves show the increase of fluorescence with different SP-BN concentrations.

FIG. 8 shows lipid transfer by SP-BN between lipid bilayers. Liposomes containing fluorescent lipids were incubated with SP-BN and then subjected to flotation in a Nycodenz gradient. Fractions were collected and analyzed for fluorescence (top panel). The bottom fractions, containing PL bound to SP-BN, were mixed with excess unlabeled liposomes and again subjected to centrifugation in a Nycodenz gradient. Fractions were analyzed for fluorescence (bottom).

FIG. 9 shows lipid transfer from commercial surfactant (CUROSURF®) to SP-BN. CUROSURF® was incubated with SP-BN for different time periods. The mixture was centrifuged through a 100K filter and the filtrate was subjected to thin-layer chromatography. Lipids were stained with Primuline.

FIGS. 10A-10B show a SP-BN mutant defective in PL binding and transfer. (FIG. 10A) Wild-type (WT) and mutant (L36K/L45E/V80K) SP-BN were analyzed for bound PL by thin-layer chromatography. The bracket indicates bound PL (PE and PG) in the WT. (FIG. 10B) Mutant SP-BN is inactive in the dequenching assay.

FIG. 11 shows crystal structures of SP-BC, obtained at high and low pH. The high pH structure is a dimer with both monomers arranged in parallel. The low pH structure is probably a tetramer. The monomers are anti-parallel in each of the two dimers that form the tetramer. A hydrophobic pocket is at the interface between the dimers.

FIG. 12 shows SP-BN is a soluble protein in rat lungs. A homogenate was subjected to sucrose gradient centrifugation and fractions were collected from the top. Note that SP-BN and SP-BM peak in fractions 1 and 4, respectively. The membranes in each fraction were subsequently pelleted. All samples were analyzed by SDS-PAGE and immunoblotting for SP-BN and SP-BM.

FIG. 13 shows pulse-chase experiments with alveolospheres. Cells were incubated for different time periods with 35S-methionine (pulse; left) or incubated after the pulse with unlabeled methionine for different time points. The samples were solubilized in detergent and subjected to immunoprecipitation with SP-BN antibodies, followed by autoradiography.

FIG. 14 shows AAV9 infection allows expression of EGFP through the lung. AAV9 virus was administered by oral instillation. Lung slices were analyzed by immuno-staining for proSP-C, a marker of alveolar type II cells, and for virally expressed EGFP.

FIG. 15 shows SP-BM purified from bovine lung and refolded after denaturation. The native dimer consists of two monomers disulfide-linked through Cys48 (upper arrow). Purified SP-BM was denatured in 8M urea and 10 mM DTT at 25° C., and refolded by dialysis against buffer containing 5 mM GSH and 0.5 mM GSSG (reduced and oxidized glutathione). Shown is the soluble fraction after centrifugation (lower arrow). All samples were analyzed by SDS-PAGE and Coomassie staining.

FIG. 16A-16D show that reconstituted SP-BM forms LB-like structures. (FIG. 16A) Purified SP-BM was mixed with DPPC/PG and the detergent was removed by dialysis. The membranes were pelleted and analyzed by thin-section EM and osmium staining. Membrane lamellae are connected to protein-rich areas. (FIG. 16B) Magnified view of the boxed area in (FIG. 16A). (FIG. 16C) As in (FIG. 16B), but 5 mM CaCl2) was added after dialysis. (FIG. 16D) As in (FIG. 16C), but different example.

FIG. 17 shows the beneficial effect of a mixture of liposomes and SP-BN in LPS-treated mice. Shown are three parameters for which statistically significant effects were seen compared to controls receiving liposomes without protein. The decrease of dead neutrophils can point to a role of SP-BN in reducing inflammation.

FIGS. 18A-18G show that the reconstituted SP-BM generates LB-like structures. (FIG. 18A) Domain structure of the SP-B precursor. Conserved cysteines (C) and disulfide bridges are indicated. (FIG. 18B) Thin-section EM of a human LB (reproduced from (Stratton, 1978)). PC, projection core; MC, matrix core. (FIG. 18C) Purification of SP-BM from bovine lungs. Fractions of the last SEC were analyzed by SDS-PAGE and Coomassie-blue staining. The position of SP-B is indicated by an arrow. (FIG. 18D) Liposomes reconstituted with bovine SP-BM (10% by weight) were analyzed by thin-section EM. The boxed area is magnified on the right. (FIG. 18E) As in (FIG. 18D) but in the presence of 5 mM CaCl2). Shown are two examples. (FIG. 18F) Purified recombinant SP-BM analyzed by reducing SDS-PAGE and Coomassie-blue staining. (FIG. 18G) Liposomes reconstituted with recombinant SP-BM (10% by weight) in the presence of 5 mM CaCl2) were analyzed by thin-section EM. Different examples are shown.

FIGS. 19A-19G show the crystal structures of SP-BN. (FIG. 19A) Side and top views of the structure of wild-type SP-BN. The two monomers are shown as cartoons in grey and red. The four helices typical for saposin-like proteins are numbered H1 to H4. The three bound PLs were modeled as PE molecules and are shown in stick representation. (FIG. 19B) As in (FIG. 19A), but with the protein in white (left) or omitted (right). (FIG. 19C) Mutated residues at the “roof”. (FIG. 19D) Mutated residues in the hydrophobic interior. (FIG. 19E) Mutated residues at the two ends of the hollow. (FIG. 19F) Superposition of the crystal structures of wild-type SP-BN (red) and the K46E/R51E mutant (blue). (FIG. 19G) As in (FIG. 19F), but for the PL molecules only.

FIGS. 20A-20F show phospholipid transfer by SP-BN. (FIG. 20A) Liposomes with an ER-like PL composition and fluorescent NBD-PE were incubated at pH 5.4 with or without purified SP-BN and floated in a discontinuous Nycodenz gradient. Fractions were analyzed by their fluorescence and by SDS-PAGE and Coomassie-blue staining (lower panel). (FIG. 20B) The two bottom fractions of the gradient in (FIG. 20A) were combined, incubated with excess unlabeled liposomes, and re-run in a discontinuous Nycodenz gradient. (FIG. 20C) Liposomes with an LB-like PL composition and either NBD-PE (donor fluorophore) or rhodamine-PE (acceptor fluorophore) were incubated with SP-BN at the indicated pH values. NBD fluorescence was followed over time and normalized with respect to fluorescence measured after detergent addition. (FIG. 20D) Liposomes with ER- or LB-like PL compositions were incubated with SP-BN for different time periods. The samples were centrifuged through a 100 kDa cut-off filter, extracted with chloroform/methanol, and analyzed by TLC. Lipids were visualized by Primulin staining. Standards were run in parallel. (FIG. 20E) As in (FIG. 20C), but with wild-type SP-BN and K46E/R51E mutant and liposomes containing either negatively charged (DPPC/PG) or neutral (DPPC/DOPE) PLs. (FIG. 20F) As in (FIG. 20C), but with SP-BN (P50C) containing or lacking a disulfide bridge that locks the “roof”. The reactions were performed at pH 5.6 with or without DTT.

FIGS. 21A-21F show lipid-binding and -transfer by SP-BN is required for proSP-B trafficking in alveolar cells. (FIG. 21A) Alveolar ILE-15 wild-type (WT) or SP-B knock-out (KO) cells were transfected with plasmids coding for mouse (m) or human (h) SP-B. mSP-B carried C-terminal Myc and Flag tags (mSP-B-Myc-Flag). Where indicated, hSP-B had three mutations (L96K/L105E/L140K) abrogating PL binding to the SP-BN domain. Cells and medium were analyzed by SDS-PAGE and immunoblotting with antibodies to mouse SP-BN (upper gel), human SP-B (middle gel), or SP-BM (lower gel). (FIG. 21B) Cells and medium of hSP-B transfected cells were treated with endoglycosidase H (Endo H; upper panel. The samples in the lower panel were extracted with 1% Triton X-100, and pellets and supernatants were analyzed by immunoblotting with human SP-B antibodies. (FIG. 21C) Cells were transfected with wild-type or triple-mutant hSP-B and analyzed by immunostaining with antibodies recognizing human proSP-B or SP-BM. Merged images are shown on the bottom. (FIG. 21D) mSP-B or the indicated mutants were expressed in KO cells. Cells and medium were analyzed by immunoblotting for proSP-B and mature SP-BM. Identical aliquots were treated with 0.1 M Na2CO3 pH 11 or 1% Triton-100 (TX-100) and the pellets were analyzed. (FIG. 21E) SP-BΔMC constructs, lacking the SP-BN and SP-BC domains, which were otherwise wild-type or carried the indicated mutations, were expressed in KO cells. Full-length hSP-B was expressed as a control. The cells and medium were analyzed by immunoblotting with human SP-B antibodies. (FIG. 21F) Full-length hSP-B or HALO-tagged hSP-B, or constructs lacking SP-BC (SP-BAC and HALO-SP-BAC, respectively), were expressed in KO cells. Control cells expressed YFP. Cells and medium were analyzed by immunoblotting for pro-SP-B and SP-BM.

FIGS. 22A-22C show ProSP-B has enhanced lipid transfer activity. (FIG. 22A) Purified proSP-B or SP-BN were tested at pH 5.4 for lipid transfer with the fluorescence de-quenching assay. The PL composition of donor and acceptor vesicles resembled that of LBs and the molar protein to lipid ratio (P/L) was varied, as indicated. The data were normalized to fluorescence measured after addition of detergent. (FIG. 22B) As in (FIG. 22A), but at pH 7.4. (FIG. 22C) As in (FIG. 22A), but with liposomes that have an ER-like lipid composition.

FIGS. 23A-23E show ProSP-B forms lipoprotein particles. (FIG. 23A) Liposomes containing a LB-like lipid composition and fluorescent NBD-PE were incubated with purified proSP-B at 370C and pH 5.6. Controls were done with liposomes and protein alone. The samples were subjected to SEC and analyzed by absorbance at 280 nm and, for fluorescent lipid, at 560 nm. Fractions of the runs with protein were analyzed by SDS-PAGE followed by immunoblotting for proSP-B (lower panels). (FIG. 23B) Liposomes were incubated with proSP-B at either 37° C. and pH 7.5 or at 0° C. and pH 5.6. The samples were subjected to SEC and fractions were analyzed by SDS-PAGE and immunoblotting. (FIG. 23C) Negative-stain EM images of the lipoprotein particle fraction indicated in (FIG. 23A). The inset shows a magnified view. (FIG. 23D) As in FIG. 23C, but with liposomes lacking protein. (FIG. 23D) As in (FIG. 23C), but with the dimer fraction of proSP-B in the absence of liposomes. The inset has the same magnification as the one in (FIG. 23C).

FIGS. 24A-24F demonstrate the phenotype of mice with mutations in the SP-BN domain. (FIG. 24A) Scheme for the generation of CRISPR knock-in mice. The exons of the SP-B gene are numbered. The yellow arrowheads indicate the positions of the guide RNAs. The donor DNAs carrying the desired mutations are shown above and below the SP-B gene, with the mutations indicated as stars. An intron was deleted to generate the triple-mutant. (FIG. 24B) CRISPR knock-in with the triple-mutant L95K/L104E/V139K. The genotype of embryos was determined and proteins were analyzed by SDS-PAGE and immunoblotting with SP-BN and SP-BM antibodies. INDEL, insertion/deletions in both alleles. (FIG. 24C) The knock-in of the triple-mutant was repeated and newborn mice were genotyped and assessed for viability. (FIG. 24D) As in (FIG. 24B), but for the double mutant (K105E/R110E). The middle panel shows a Coomassie-stained gel to demonstrate equal loading. The lowest panel shows a titration of the wild-type (WT) sample, which was used to estimate that the mutant makes ˜10% of wild-type levels of SP-BM. (FIG. 24E) As in (FIG. 24C), but for the double mutant. (FIG. 24F) Hypothetical mechanism of LB formation by SP-B.

FIGS. 25A-25D (related to FIGS. 18A-18G). show that reconstituted, purified SP-BM forms LB-like structures. (FIG. 25A) SP-BM purified from bovine lung was reconstituted into liposomes containing a LB-like lipid composition at different weight percentages of protein. The samples were analyzed by thin-section EM. The insets show magnified views of the boxed areas. (FIG. 25B) As in (FIG. 25A), but with 10% (w/w) SP-BM and liposomes that contain an LB-like or ER-like lipid composition. The latter does not show projection cores with emanating membrane bubbles. (FIG. 25C) As in (FIG. 25B), but with high- or low-molecular weight (MW) fractions obtained by SEC of recombinant SP-BM (these are controls for the dimer fraction analyzed in FIG. 18G). (FIG. 25D) As in (FIG. 25B), but with purified proSP-B. No projection cores with emanating membrane bubbles are visible.

FIGS. 26A-26D show purification and characterization of recombinant SP-BM. (FIG. 26A) Scheme of the construct used to generate recombinant SP-BM. The construct contains GST at the N-terminus and lacks SP-BC and the signal sequence. A thrombin cleavage site precedes the SP-BM domain. (FIG. 26B) Construct was expressed in E. coli and formed inclusion bodies. After cell lysis, the extract was centrifuged to generate a pellet (P) and supernatant (S). The pellet was washed twice and solubilized in sarkosyl (sarkosyl extr.). After reduction of the sarkosyl concentration, the protein was cleaved with thrombin. All samples were analyzed by SDS-PAGE and Coomassie-blue staining. The arrow indicates the position of SP-BM. (FIG. 26C) After thrombin cleavage, the sample was extracted with chloroform/methanol and subjected to chromatography on Sephadex LH20. The indicated fractions were pooled. (FIG. 26D) After removal of the organic solvent, the sample was solubilized in octylglucoside and subjected to SEC on a Superdex 200 Increase column. Fractions were analyzed by non-reducing and reducing SDS-PAGE, followed by Coomassie staining. The star and arrow indicate the positions of the dimer of SP-BM, containing an inter-molecular disulfide bond, and of the monomer, respectively. The right panel shows purified SP-BM analyzed by reducing SDS-PAGE the Superdex 200 separation of SP-BM. For comparison, SP-BN was analyzed. Note that all samples contain a high percentage of α-helical structure. (FIGS. 26E-26F) Purified SP-BM was subjected to mass spectrometry (sequence in red). The confirmed sequence covers ˜71% of SP-BM.

FIGS. 27A-27D (related to FIGS. 19-20) show lipid-binding and -transfer by SP-BN. (FIG. 27A) Wild-type (WT) SP-BN or the indicated mutants were purified after expression in E. coli. The bound PLs were extracted with chloroform/methanol and analyzed by TLC. PE and PG standards were run in parallel. The star indicates an unidentified species. (FIG. 27B) Lipid transfer by SP-BN using fluorescent donor (NBD-PE) and acceptor (rhodamine-PE) vesicles of different sizes. The vesicles were extruded through filters of the indicated diameters. The increase of NBD fluorescence was followed over time and normalized to that observed after addition of detergent. (FIG. 27C) SP-BN was tested for lipid transfer in the fluorescence de-quenching assay using donor and acceptor liposomes of the indicated lipid composition. The ratio of PC to the other PLs in the mix was 3:1. (FIG. 27D) Wild-type (WT) SP-BN or the indicated mutants were tested for lipid transfer using the fluorescence de-quenching assay. (FIG. 27E) Lipid was removed from SP-BN with 6% octylglucoside. The sample was dialyzed and subjected to lipid extraction and TLC (inset) or SEC. As a control, the original sample was analyzed. (FIG. 27F) As in (FIG. 27C) but with the Y59A/H79A mutant of SP-BN. (FIG. 27G) SP-BN was tested for lipid transfer at the indicated molar ratios of protein to lipid (P/L).

FIGS. 28A-28B (related to FIGS. 19-20) show the crystal structure of the Y59A/H79A mutant of SP-BN. (FIG. 28A) The middle panel shows the four dimers in the asymmetric unit in cartoon representation of different colors. The panels on the sides highlight the position of the PLs. One dimer copy is in the back and thus not visible. Magnified views show PL molecules located at the interface between dimers. (FIG. 28B) PL molecules in the four dimer copies. For each dimer copy, a top view is shown (roof in the foreground) with PL molecules in color and the protein in a white cartoon model. In addition, a side view of the PLs is shown without protein.

FIGS. 29A-29C (related to FIG. 20) shows the crystal structures of SP-BC. (FIG. 29A) Crystal structure of SP-BC obtained at pH 8.5. The asymmetric unit contains a dimer. The two monomers in the dimer are shown as cartoons in different colors and the N- and C-termini are labeled. (FIG. 29B) Crystal structure obtained at pH ˜4.5. The asymmetric unit contains two tetramers, each consisting of two dimers, which are colored differently. (FIG. 29C) Cavity at the interface between the two dimers, calculated with the program CavityPlus (Xu et al., 2018). The dimer interface in the tetramer is indicated by a line. The right panel shows a slice through the space-filling model of the region containing the cavity. Residues are colored according to their hydrophobicity (scale on the right).

FIGS. 30A-30E (related to FIG. 21) show the expression of wild-type and mutant SP-B in wild-type and knock-out MLE-15 cells. (FIG. 30A) Scheme for the generation of a CRISPR knock-out of SP-B in MLE-15 cells. Shown is the SP-B gene with exons numbered. The yellow arrowheads indicate the positions of the guide RNAs. The indicated region was deleted. The lower panel shows the sequence across the exon 4/5 junction, with the guide RNA region underlined. PAM, proto-spacer adjacent motif (FIG. 30B) Membranes from wild-type (WT) ILE-15 and SP-B knock-out (KO) cells were subjected to flotation in a sucrose gradient. Fractions were analyzed by SDS-PAGE followed by immunoblotting with SP-BM antibodies (upper panel) and Coomassie-blue staining (lower panel). (FIG. 30C) Human SP-B (hSP-B) was expressed in WT cells. The membrane fraction and a chloroform/methanol extract of the membranes were analyzed as in (FIG. 30B). (FIG. 30D) Full-length proSP-B, proSP-B with a HALO-tag at its N-terminus (HALO-full-length), or constructs lacking the region following SP-BM (AC and HALO-AC, respectively), or triple-mutant proSP-B (L95K/L104E/V139K) were expressed in KO cells. A control was performed with YFP-expressing cells. Aliquots of the medium and cell pellet were treated with endoH, as indicated, and analyzed by SDS-PAGE followed by immunoblotting with proSP-B antibodies (upper panel). Other aliquots were treated with Triton X-100, and the soluble and insoluble fractions were analyzed by SDS-PAGE and immunoblotting (lower panel). The TX-100 soluble fraction probed with proSP-B antibodies is also shown in FIG. 21F. (FIG. 30E) Mouse SP-B (mSP-B) carrying the K105/R110E mutations, which affect lipid transfer by the SP-BN domain, was co-expressed with human SP-B (hSP-B) or hSP-B lacking the SP-BM and SP-BC domains (SP-ΔMC), as indicated. The medium and cell pellet were analyzed by SDS-PAGE and immunoblotting with antibodies to mouse SP-BN, human SP-B, or SP-BM.

FIGS. 31A-31G (related to FIGS. 22-24) show the purification of proSP-B and thin-section EM of alveolar type II cells expressing wild-type or mutant proSP-B. (FIG. 31A) ProSP-B was secreted as a HALO-tagged fusion protein from HEK293 cells. The protein was purified with a HALO resin and the tag was proteolytically removed. The purified protein was analyzed by SDS-PAGE and Coomassie-blue staining. (FIG. 31B) Lipids bound to purified proSP-B were extracted with chloroform/methanol and separated by TLC. A mock sample was treated analogously. The question mark indicates unidentified, relatively hydrophilic, lipids. (FIG. 31C) A lung of a wild-type mouse embryo was analyzed by thin-section EM. The inset shows a magnified view of the boxed area. (FIG. 31D) As in (FIG. 31C), but for an embryo with a triple mutation in the SP-BN domain. Note the absence of LBs. (FIG. 31E) As in (FIG. 31C), but for an embryo that carries insertion/deletions (INDEL) in both alleles. Note the absence of LBs. (FIG. 31F) As in (FIG. 31C), but for an embryo with a double mutation in the SP-BN domain. Normal LBs are visible. (FIG. 31G) As in (FIG. 31C), but for an embryo with a single mutation in the SP-BN domain. Normal LBs are visible.

FIGS. 32A-32F show additional views of the crystal structures of SP-BN. See also FIG. 19A-19G. (FIG. 32A) Structure of wild-type SP-BN in two different views. The two monomers are shown as cartoons in different shades of blue. The bound lipids are in white. The four helices typical for saposin-like proteins are numbered. Disulfides are labeled in yellow, and the positions of the “floor” and “roof” are indicated. (FIG. 32B) Two different views of the three PLs (in color) bound to SP-BN (white). The PLs were modeled as the most abundant PE species, i.e. PE with acyl chains of 16 and 18 carbon atoms, each containing one double-bond (Table 2). Because the composition of bound PLs is heterogeneous, the density for the PL head groups is ill-defined. (FIG. 32C) Mutated residues at the “roof”. (FIG. 32D) Mutated residues in the hydrophobic interior and at the two ends of the hollow. Shown is a cut-away view, with labels on one monomer. (FIG. 32F) Superposition of the crystal structures of wild-type SP-BN (blue) and the K46E/R51E mutant (yellow). (FIG. 32G) As in (FIG. 32F), but for the PL molecules only

FIGS. 33A-33C show the controls for SP-BM reconstitutions and predicted structure of SP-BM, Related to FIG. 25. (FIG. 33A) High- or low-molecular weight (MW) fractions obtained by SEC of recombinant SP-BM were reconstituted into liposomes at 10% weight in the presence of 5 mM CaCl2), and analyzed by thin-section EM. These images show no projection cores with emanating membrane layers and serve as control for the dimer fraction analyzed. (FIG. 33B) As in (FIG. 33A), but with purified proSP-B. Again, no projection cores with emanating membrane bubbles are visible. (FIG. 33C) Predicted structure of SP-BM according the program Swiss-model (Waterhouse et al., 2018). The left panel shows a cartoon model and the right panel a space filling model, with residues colored according to their hydrophobicity (scale on the right). It is contemplated that the surface facing the viewer can interact with the lipid bilayer edge.

### DETAILED DESCRIPTION

The compositions and methods provided herein are related, in part, to the discovery that N-terminal and middle domains of surfactant protein B or SP-B (SP-BN and SP-BM) function together and separately in LB formation in alveolar cells. Thus, provided herein are methods of making SP-BN and SP-BM for the treatment of a respiratory disease in a subject in need thereof. In particular, it was discovered that SP-M can be made by expressing a fusion protein comprising both SP-BN, a cleavage linker, and SP-BM in a cell, solubilizing the admixture, renaturing the fusion protein, and purifying the SP-BM protein.

Prior to the methods provided herein the expression and purification of recombinant SP-BM was technically challenging. It was contemplated that the extreme hydrophobicity of SP-BM made the protein difficult to isolate and predict its structure by standard crystallization techniques. The methods provided herein have made it possible to express and purify the two SP-B domains, SP-BN and SP-BM recombinantly. The methods provided herein permit the purification and isolation of each individual SP-B domain, including a SP-BN domain, a SP-BM, domain, a SP-BC domain or any combination thereof.

### Lung Surfactants:

Pulmonary surfactant is a mixture of proteins and phospholipids secreted by alveolar type II epithelial cells in the lung. Surfactant forms a thin extracellular layer, which drastically reduces surface tension, thus facilitating breathing and preventing alveolar collapse. The air-liquid interface is thought to be lined by a monolayer of phospholipids, which is derived from a lipid bilayer that is initially stored inside the cells in lamellar bodies (LBs). LBs are lysosome-like organelles, in which membrane sheets are densely stacked on top of each other like onion layers. Upon stimulation of exocytosis, the limiting membranes of LBs fuse with the plasma membrane, releasing the internal membrane sheets to the extracellular space.

The methods and working examples provided herein address the mechanism by which mammalian surfactant protein B (SP-B) domains function in respiration. Surfactant B protein or “SP-B” is a lipid-associated protein found in pulmonary surfactant that is essential for breathing in mammals such as mic and humans. SP-B is a conserved protein that is found in all animal species with lungs. In humans, there are rare homozygous cases in which the gene for SP-B is mutated and the protein is not made, resulting in severe respiratory problems that generally require lung transplantation within a few months after birth. SP-B is not only involved in reducing the surface tension as a component of the extracellular surfactant, but is also essential for the formation of lamellar bodies in these cells.

SP-B is made in alveolar type II cells as a precursor containing three domains related to the lysosome-localized saposins. The SP-B polypeptide precursor domains are the N-terminal domain (SP-BN), the middle domain (SP-BM), and the C-terminal domain (SP-BC). The human amino acid and nucleic acid sequences for each domain are known in the art and non-limiting examples of the amino acid and nucleic acid sequences are provided herein. The full-length amino acid sequence of SP-B is provided herein as SEQ ID NO: 1. Non-limiting examples of the amino acid sequences of each SP-B domain are provided herein as SEQ ID NO: 2 (SP-BN), SEQ ID NO: 3 (SP-BM), and SEQ ID NO: 6 (SP-BC). The human nucleic acid sequences encoding the SP-B protein can be found on NCBI as NCBI Reference Gene ID: 6439 (SEQ ID NO: 7). SP-B mRNA transcripts are provided herein as SEQ ID NO: 8, SEQ ID NO: 9, and SEQ ID NO: 10. Non-limiting examples of additional isoforms of the SP-B polypeptide are also known in the art and include the amino acid sequences of SEQ ID NO: 11 and SEQ ID NO: 12.

The precursor of SP-B is proteolytically processed into the individual domains, with the middle domain (SP-BM) currently considered to be the biologically important “mature” protein responsible for surfactant function. SP-BM is initially stored in lamellar bodies (LB), lysosome-like organelles, in which membrane sheets are densely stacked on top of each other. LBs are eventually exocytosed to generate extracellular surfactant, a mixture of phospholipids and protein. The exported lipid bilayers are then transformed into a lipid monolayer at the air-water interface, which reduces surface tension and facilitates breathing. Provided herein are biochemical, structural, and cell biological methods and compositions used to express, purify, and characterize the N-terminal and middle domains of SP-B (SP-BN and SP-BM, respectively). The molecular mechanisms by which these domains function together in LB formation are also elucidated and demonstrated in the working examples.

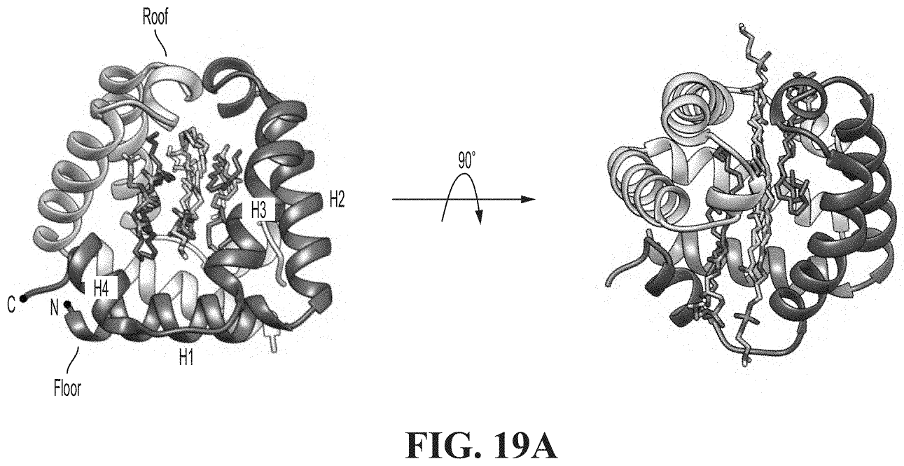

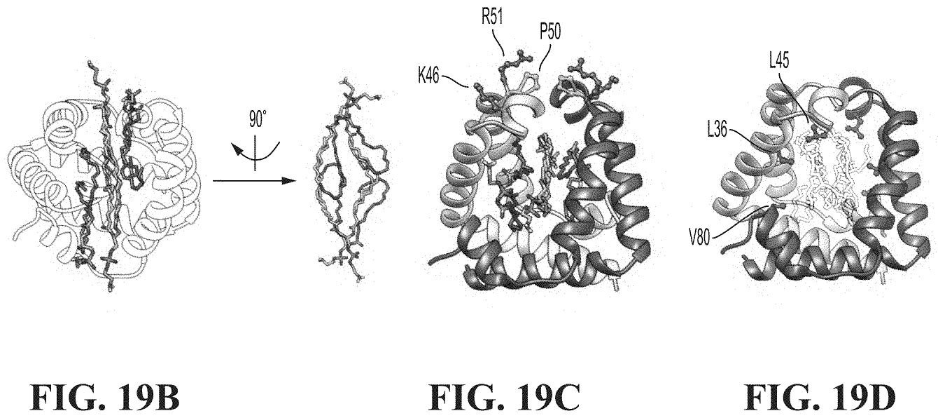

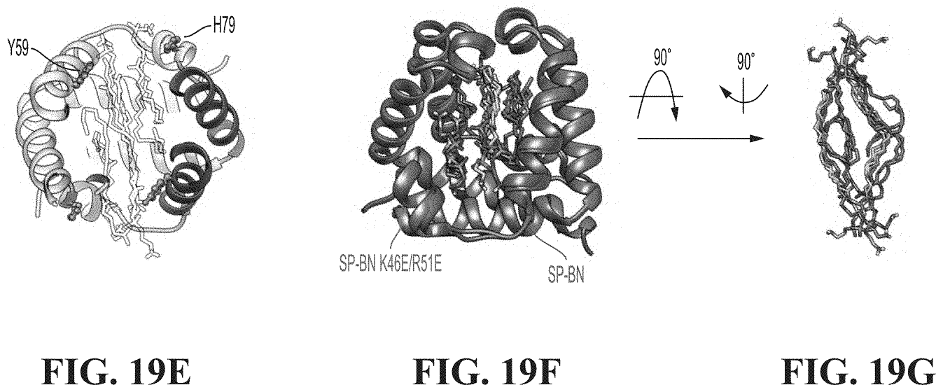

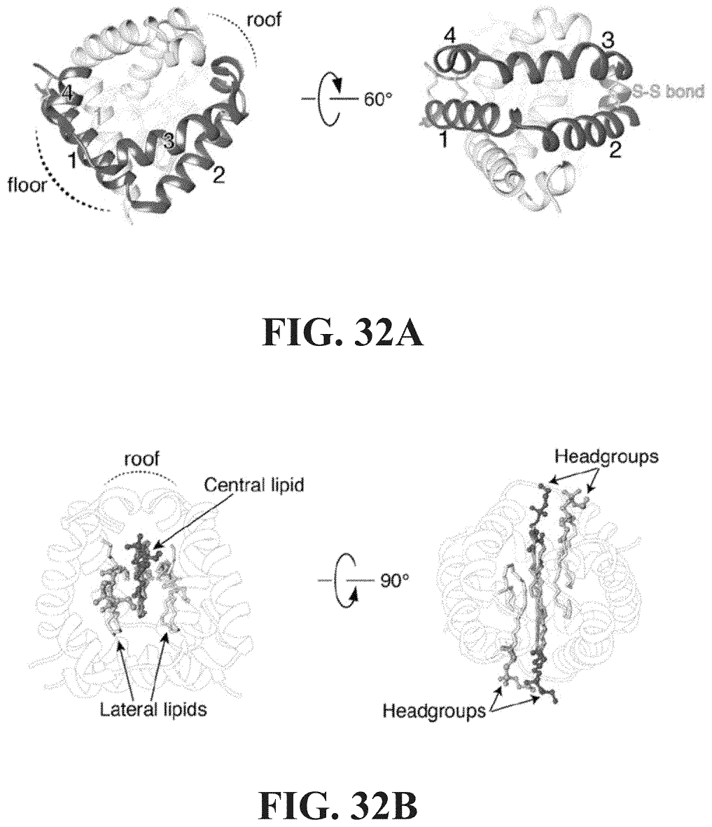

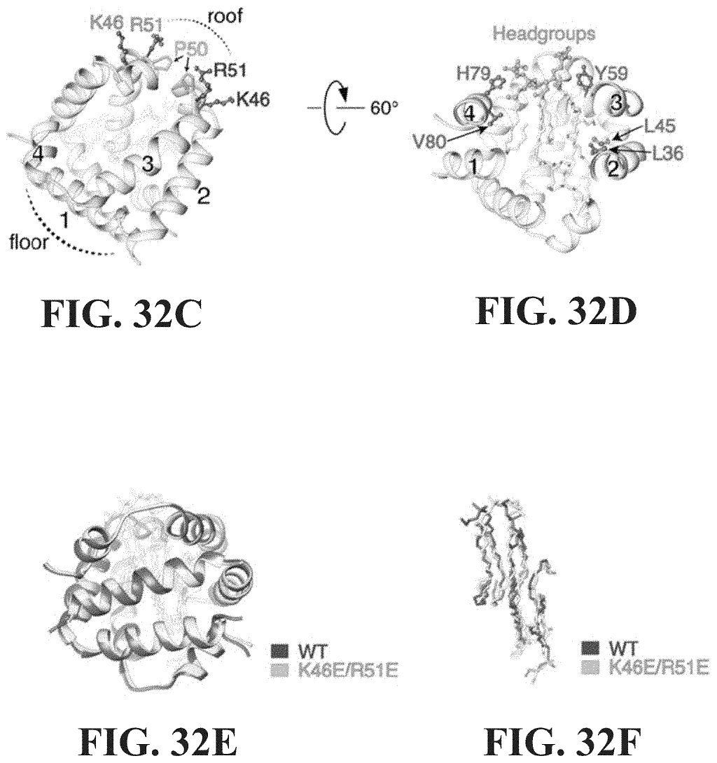

As provided herein in the working examples, the SP-BN monomer consists of four helices with conserved intramolecular disulfide bridges and a loop between helices 2 and 3 (FIG. 19A, FIG. 32A). In the SP-BN, two monomers in an open conformation form a dimer with a hydrophobic hollow in between. The monomers are associated through their helices 1 at the “floor”. The two loops between helices 2 and 3 contain additional short helices and form a “roof”. Inside the hydrophobic hollow are three phospholipid molecules that originate from the heterologous cells in which the protein was expressed. Thus, the lung surfactant compositions provided herein can comprise one or more domains of the SP-B polypeptide or the polypeptide domains provided herein can form dimers or macromolecules.

In one aspect, provided herein is a lung surfactant composition for use in the treatment of a respiratory disease. In another aspect, the lung surfactant composition comprises an SP-BN domain; and a phospholipid. In another aspect, the lung surfactant composition comprises at least one SP-BN domain; at least one SP-BM domain; and a phospholipid. In yet another aspect, the lung surfactant composition comprises at least one SP-BM domain; and a phospholipid.

In some embodiments of any of the aspects, the lung surfactant composition, polypeptides, and fusion proteins provided herein further comprise a SP-BN, a SP-BM, and/or a SP-BC domain. The lung surfactant composition can comprise the SP-B domains as separate polypeptides or as a single polypeptide. In some embodiments, the polypeptide is recombinant. For example, the lung surfactant composition comprises: (a) a polypeptide comprising in an N-terminal to C terminal direction: (1) an SP-BN domain and (2) a SP-BM domain; and (b) at least one phospholipid. As another example, the lung surfactant composition can comprise: (a) a polypeptide comprising in an N-terminal to C-terminal direction: (1) a SP-BM domain and (2) a SP-BN domain; and (b) at least one phospholipid.

In some embodiments, the lung surfactant composition comprises separate SP-B domains. For example, the lung surfactant comprises: (a) a polypeptide comprising a SP-BM domain; (b) at least one phospholipid; and (c) a polypeptide comprising a SP-BN domain.

In some embodiments of any of the aspects, the composition comprises one or more, two or more, three or more, or four or more SP-BN domains. In some embodiments of any of the aspects, the composition comprises, one or more, two or more, three or more, or four or more SP-BM domains. In some embodiments of any of the aspects, the composition comprises one or more, two or more, three or more, or four or more SP-BN domains and one or more, two or more, three or more, or four or more SP-BM domains. In some embodiments of any of the aspects, the composition comprises one or more polypeptide linkers.

In some embodiments of any of the aspects, a “peptide” or “polypeptide” or “domain” as described herein (or a nucleic acid encoding such a polypeptide or domain) can be a functional fragment of one or more portions of the SP-B polypeptides, domains, or amino acid sequences provided herein. The functional fragment can be assessed by any method known in the art. For example, by lamellar body formation or increase respiratory function when administered to an animal model (e.g., a rodent or primate, or a model of a respiratory disease).

In some embodiments of any of the aspects, the composition comprises a mutation in one or more SP-B domains.

In some embodiments of any of the aspects, the composition provided herein is a fusion protein. For example, the composition can comprise a fusion of at least one SP-BN polypeptide domain with at least one SP-BM polypeptide domain, wherein the fusion protein lacks a C-terminal domain (SP-BC).

The fusion protein can be engineered with a polypeptide tag for purification as provided herein. The fusion protein can further be engineered to have a protease cleavage site for engineering a protein recombinantly in a facile manner. The fusion protein provided herein can comprise, for example, a His-tag, FLAG, HALO, GST, SP-BN, and/or SP-BM for expression in a heterologous cell (e.g. bacterium or mammalian cell). A non-limiting example of a fusion protein sequence is provided in SEQ ID NO: 4 and SEQ ID NO: 16.

In some embodiments of any of the aspects, the composition comprises a protease cleavage site. In another embodiment of any of the aspects, the protease cleavage site is a tobacco etch virus (TEV) protease or thrombin site (SEQ ID NO: 5). In another embodiment of any of the aspects, the composition comprises SEQ ID NO: 4.

It is contemplated that other cleavable linkers can be used. A linker can include a cleavable linking group that is cleavable by a particular enzyme. The type of cleavable linking group incorporated into a linker can depend on the cell to be targeted.

In some embodiments, the composition provided herein further comprises a peptide linker. Peptide linkers may affect folding of a given fusion protein, and may also react/bind with other proteins, and these properties can be screened for by known techniques. Example linkers, in addition to those described herein, include is a string of histidine residues, e.g., His6; sequences made up of Ala and Pro, varying the number of Ala-Pro pairs to modulate the flexibility of the linker; and sequences made up of charged amino acid residues e.g., mixing Glu and Lys. Flexibility can be controlled by the types and numbers of residues in the linker. See, e.g., Perham, 30 Biochem. 8501 (1991); Wriggers et al., 80 Biopolymers 736 (2005). Chemical linkers may comprise a direct bond or an atom such as oxygen or sulfur, a unit such as NH, C(O), C(O)NH, SO, SO₂, SO₂NH, or a chain of atoms, such as substituted or unsubstituted C₁-C₆ alkyl, substituted or unsubstituted C₂-C₆ alkenyl, substituted or unsubstituted C₂-C₆ alkynyl, substituted or unsubstituted C₆-C₁₂ aryl, substituted or unsubstituted C₅-C₁₂ heteroaryl, substituted or unsubstituted C₅-C₁₂ heterocyclyl, substituted or unsubstituted C₃-C₁₂ cycloalkyl, where one or more methylenes can be interrupted or terminated by O, S, S(O), SO₂, NH, or C(O). The linker domain can be 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond.

The compositions provided herein can comprise a tag, label, or bead for purification or visualization in a cellular system. In some embodiments, the tag provided herein is conjugated to the C-terminus of the protein. In some embodiments, the polypeptide tag is conjugated to the N-terminus of the protein. In some embodiments, SP-BN or SP-BM is further conjugated to a bead for purification. In some embodiments of any of the aspects, the polypeptides provided herein are conjugated to a fluorescent label.

Such proteins can further facilitate purification, tracking and/or visualization of the SP-BN and SP-BM proteins. Non-limiting examples of heterologous protein tags that can be used include Histidine (HIS), sequence motif DYKDDDDK (where D=aspartic acid, Y=tyrosine, and K=lysine) or a FLAG tag, β-galactosidase, human influenza hemagglutinin (HA), OLLAS, c-Myc, paramyxovirus of simian virus 5 epitope (V5), HALO, streptavidin-binding peptide (SBP), maltose binding protein (MBP), SUMO, or any other protein epitope tag known in the art.

In the various embodiments of any of the aspects, it is contemplated that variants (naturally occurring or otherwise), alleles, homologs, conservatively modified variants, and/or conservative substitution variants of any of the particular polypeptides described are encompassed. As to amino acid sequences or fragments thereof (e.g. SEQ ID NO: 1, SEQ ID NO: 11, and SEQ ID NO: 12), one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

A variant amino acid or DNA sequence can be at least 80%, at least 90%, at least 91%, at least 92%, at least 93%, at least 94%, at least 95%, at least 96%, at least 97%, at least 98%, at least 99%, identical to a native or reference sequence (e.g. NCBI Reference sequence Gene ID: 6439). The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence (e.g. of SP-B, SP-BM, or SP-BN) can be accomplished by any of a number of techniques known in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

As provided herein, the lung surfactant composition comprises a phospholipid (also abbreviated herein as PL). A phospholipid is a class of lipid that consists of hydrophilic head with a phosphate group and two hydrophobic fatty acid tails. The structure of the phospholipid molecule permits the formation of lipid bilayers due to these amphipathic characteristics.

In some embodiments of any of the aspects, the phospholipid is a glycerophospholipid. A glycerophospholipid is a phospholipid comprising any derivative of glycerophosphoric acid. For example, the glycerophospholipid can comprise at least one O-acyl, or O-alkyl, or O-alk-1′-enyl residue attached to the glycerol moiety.

In some embodiments of any of the aspects, the phospholipid is selected from the group consisting of: 1,2-dipalmitoyl-sn-glycero-3-phosphocholine (DPPC); phosphatidylglycerol (PG); phosphatidylcholine (PC), phosphatidylethanolamine (PE), phosphatidylserine (PS), and phosphatidylinositol (PI), or any combination thereof.

In another embodiment of any of the aspects, the composition provided herein comprises DPPC and/or PG. In another embodiment of any of the aspects, the composition provided herein comprises a 1:1 ratio, a 2:1 ratio, a 3:1 ratio, a 4:1 ratio, a 5:1 ratio or more of DPPC to PG. In another embodiment of any of the aspects, the composition provided herein comprises a 1:1 ratio, a 2:1 ratio, a 3:1 ratio, a 4:1 ratio, a 5:1 ratio or more of PG to DPPC.

In another embodiment of any of the aspects, the composition provided herein comprises PE and PG. In another embodiment of any of the aspects, the composition provided herein comprises a 1:1 ratio, a 2:1 ratio, a 3:1 ratio, a 4:1 ratio, a 5:1 ratio or more of PE to PG. In another embodiment of any of the aspects, the composition provided herein comprises a 1:1 ratio, a 2:1 ratio, a 3:1 ratio, a 4:1 ratio, a 5:1 ratio or more of PG to PE.

In another embodiment of any of the aspects, the composition comprises one or more phospholipids that are lipase resistant. A lipase is any enzyme that catalyzes the hydrolysis of a lipid. Thus, it is contemplated herein that any phospholipid that is resistant to hydrolysis by lipases can be used in the compositions provided herein.

### Methods of Preparing Lung Surfactant Compositions

Provided herein are methods of preparing and purifying the middle domain of SP-B, SP-BM polypeptide and the N-terminal domain of SP-B, SP-BN polypeptide.

The polypeptides provided herein can be isolated directly from an organism or produced in an organism. When the polypeptides provided herein are isolated directly from an organism, the polypeptides will be obtained from a sample of biological tissue or from a plurality of organisms. In some embodiments, the biological tissue is a lung tissue from a mammal. The lung tissue can be homogenized and then solubilized in a buffer that permits separation of surfactant proteins and lipids. The source of the biological tissue can be any organism or subject that produces SP-B polypeptide or a fragment thereof. Non-limiting examples of sources include but are not limited to, mammals, humans, primates, cows, rodents (e.g., mice or rats), dogs, cats, guinea pigs, hamsters, rabbits, horses, and invertebrates (e.g. worms).

The biological sample comprising a surfactant can be homogenized and solubilized for phase separation. The organic phase can be collected after phase separation and passed through a column (e.g., gel filtration column) to separate protein from lipids. The protein fractions can be dried and dissolved in a buffer, e.g., octylglucoside (OG). This step is different from previous purification protocols, e.g., those described in Tao et al. BioTechniques 48, 61-64 (2010), which is incorporated herein by reference in its entirety).

The methods provided herein are based on characterization of SP-BM and SP-BN from animal models (e.g., rat lungs) provided herein in the working examples. The solubilized material (e.g, solubilized in OG) can then be subjected to an addition step of phase separation (e.g., gel filtration), resulting in fractions that contain essentially only SP-BM, SP-BN, or any other polypeptide provided herein.

The SP-BM and/or SP-BN polypeptides provided herein can be engineered and expressed in a cell by the methods provided herein.

In one aspect, provided herein is a method of preparing a SP-BM polypeptide, the method comprising:

a. expressing a fusion protein in a cell, the fusion protein comprising:
i. a peptide tag;
ii. a SP-BN domain or fragment thereof;
iii. a linker; and
iv. a SP-BM domain or a fragment thereof;

b. cleaving the linker; and

c. purifying the SP-BM.

In another aspect, provided herein is a polypeptide prepared by the methods provided herein.

The method of making the SP-BM polypeptide comprises preparing and expressing a fusion protein. Methods of generating fusion proteins are known in the art. The fusion protein provided herein comprises: (1) a peptide tag; (2) a SP-BN domain or fragment thereof; (3) a linker; and (4) a SP-BM domain or a fragment thereof. The amino acid sequences of the SP-B domains are provided herein.

In some embodiments of any of the aspects, the fusion protein further comprises a saposin polypeptide or a fragment thereof. In some embodiments of any of the aspects, the fusion protein further comprises a saposin polypeptide between the peptide tag and the SP-BN domain. In some embodiments, the saposin polypeptide is Saposin A (SapA).

In some embodiments of any of the aspects, the fusion protein lacks a surfactant B protein C-terminal domain (SP-BC).

In some embodiments of any of the aspects, the linker comprises a cleavage site. In some embodiments of any of the aspects, the cleavage site is a protease cleavage site. In some embodiments of any of the aspects, the cleavage site is selected from the group consisting of: a tobacco mosaic virus (TEV) protease cleavage site, a thrombin cleavage site, a factor Xa cleavage site, a 3C protease site, and a specific nickel-assisted cleavage (SNAC)-tag.

The linker (e.g., a protease cleavage site) provided herein can be introduced between SP-BN and SP-BM to allow for subsequent purification of the cleaved-off SP-BM.

The fusion protein provided herein comprises a peptide tag for purification of the SP-BM and/or the SP-BN polypeptide. In some embodiments of any of the aspects, the peptide tag is selected from the group consisting of: a glutathione S-transferase (GST) tag, a FLAG-tag, a Myc-tag, HALO, a maltose-binding peptide, and a hexa-his peptide (6×His).

In some embodiments of any of the aspects, the fusion protein comprises an amino acid sequence of SEQ ID NO: 4 or a fragment thereof. In some embodiments of any of the aspects, the fusion protein comprises an amino acid sequence of SEQ ID NO: 16 or a fragment thereof.

In another aspect, provided herein is a nucleic acid encoding the fusion protein or a nucleic acid sequence encoding the amino acid sequence of SEQ ID NO: 4 or SEQ ID NO: 16.

In some embodiments of any of the aspects, the fusion protein provided herein or the lung surfactant composition is expressed in a vector. In some embodiments, the vector is an expression vector. The nucleic acid sequences expressed will often, but not necessarily, be heterologous to a cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free host cells. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

In some embodiments of any of the aspects, the composition is comprised or encoded in a viral vector. A viral vector is a nucleic acid construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In some embodiments, the fusion protein is heterologous. As used herein, “heterologous” refers to a genetic element or polypeptide that is not naturally found or expressed in a given cell, tissue or organism. A genetic element that encodes a gene or polypeptide that is naturally found in a given cell, but that places the expression of the polypeptide under control of different regulatory elements, e.g., to make it inducible or to overexpress it, is also considered heterologous as the term is used herein.

In some embodiments, the nucleic acid constructs encoding the fusion polypeptide provided herein are encoded by one vector. In some embodiments, the nucleic acids provided herein are encoded by multiple vectors (e.g., SP-BN or SP-BM in separate vectors). Without limitation, multiple expression vectors can be used and expressed to make the polypeptides provided herein comprising the SP-B polypeptide domains as provided herein.

The method of making SP-BM comprises a step of expressing the fusion protein in a cell. Thus, the cell can be contacted with a nucleic acid or expression vector encoding the fusion protein provided herein. Non-limiting examples of cells that can be used include a bacterial cell, yeast cell, insect cell, amphibian cell, or a mammalian cell. In particular, a heterologous cell can be derived from Escherichia coli, Streptomyces, Saccharomyces cerevisiae, Spodoptera frugiperda, Xenopus laevis oocytes, human embryonic kidney (HEK) 293 cells, mouse alveolar epithelial cell line (MILE-15), COS-7 cells, Chinese hamster ovary (CHO) cells, mammalian fibroblasts, mammalian endothelial cells, mammalian epithelial cells, and the like.

Methods and compositions for administering, delivering, or contacting a cell with a nucleic acid are known in the art, e.g., liposomes, nanoparticles, exosomes, nanocapsules, conjugates, alcohols, polylysine-rich compounds, arginine-rich compounds, calcium phosphate, microvesicles, microinjection and electroporation. An “agent that increases cellular uptake” is a molecule that facilitates transport of a molecule, e.g., nucleic acid, or peptide or polypeptide, or other molecule that does not otherwise efficiently transit the cell membrane across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), or a polyamine (e.g., spermine). Further examples of agents that increase cellular uptake are disclosed, for example, in Winkler (2013). Oligonucleotide conjugates for therapeutic applications. Ther. Deliv. 4(7); 791-809. Assays known in the art can be used to test the efficiency of nucleic acid delivery. Efficiency of introduction of the polypeptides provided herein can be assessed by one skilled in the art by measuring mRNA and/or protein levels of the desired transgene (e.g., via reverse transcription PCR, western blot analysis, mass spectrometry, and enzyme-linked immunosorbent assay (ELISA)). In some embodiments, a vector or fusion protein provided herein comprises a reporter protein that can be used to assess the expression of the desired transgene, for example by examining the expression of the reporter protein by fluorescence microscopy or a luminescence plate reader.

The purification of the SP-BM can be performed by methods known in the art. The methods provided herein comprise isolating and purifying the polypeptides provided herein. By way of example only, after expression of the fusion protein provided herein, the cells can be collected by centrifugation and proteins can then be purified by methods known in the art (e.g., column purification). Generally, the SP-BM can be purified using affinity chromatography.

Affinity chromatography (also called affinity purification) is a method of separating protein fractions by specific binding interactions between molecules. A particular molecule can be chemically immobilized or conjugated to a tag that forms an affinity pair with a solid support.

As used herein, the term “affinity pair” refers to a pair of moieties that specifically bind each other with high affinity, generally in the low micromolar to picomolar range. When one member of an affinity pair is conjugated to a first element and the other member of the pair is conjugated to a second element, the first and second elements will be brought together by the interaction of the members of the affinity pair.

As used herein, the term “conjugated to” encompasses association of fusion protein with a solid support, or a member of an affinity pair by covalent bonding, including but not limited to cross-linking via a cross-linking agent, or by a strong non-covalent interaction that is maintained under conditions in which the conjugate is to be used.

In certain embodiments, the fusion protein provided herein is bound directly or indirectly to a solid support. As described herein, a “solid support” is a structure upon which one or more polypeptides (e.g., a fusion polypeptide or a fragment thereof) can be bound for cleavage of the desired SP-B domain (e.g., SP-BM). A solid support provides a ready means for isolating or removing bound portions of the fusion protein provided herein (e.g., GST-SapA-SP-BN) and isolating the desired SP-B domain fraction (e.g., SP-BM). A solid support can be in the form, for example, of a particle, bead, filter or sheet, membrane, resin, scaffold, matrix, or column.

Another example of chromatography used to purify the polypeptides provided herein, includes size exclusion chromatography. Size exclusion chromatography (SEC) or gel filtration is a method in which molecules such as polypeptide are separated by size and/or molecular weight. The chromatography columns use in SEC are generally packed with porous beads composed of dextran, agarose, or polyacrylamide. For the methods provided herein, SEC is used to purify the smaller SP-BM domain (e.g., with a 3.5 kDa membrane) by separating the SP-BM from the larger portion of the fusion protein comprising the peptide tag and the SP-BN.

The fusion proteins and polypeptides provided herein can be admixed with a detergent to form inclusion bodies. The polypeptide can be solubilized in a detergent (e.g., sarkosyl) that can be replaced by second detergent (e.g. Triton X-100 and CHAPS), which allows refolding of the polypeptide tag (e.g. GST) and purification of the desired polypeptide on a resin (e.g., SP-BM). For example, a heterologous cell system expressing the fusion protein provided herein can be isolated by centrifugation, solubilized, and renatured.

To reconstitute SP-BM and/or SP-BN into liposomes, phospholipids (e.g., DPPC and/or PG) can be dried and then added to SP-BM and/or SP-BN in an admixture (e.g., supplemented with octylglucoside (OG)). The mixture can be diluted in buffer without detergent and dialyzed against buffer for several days (e.g., 1-5 days). The admixture can also be supplemented with calcium chloride (CaCl₂)) and/or chelators such as ethylenediaminetetraacetic acid (EDTA). The sample can also be centrifuged and the pellet used for thin-section electron microscopy (EM) for analysis and the formation of lamellar bodies.

The amount of phospholipids used in the methods and compositions provided herein can depend on the yield, amount, or concentration of the protein isolated. For example, for each 1 milligram (mg) of polypeptide, 10 mg of one or more phospholipids can be added to the admixture, leaving the ratio of about 1:10 polypeptide to phospholid(s). In some embodiments, the lung surfactant composition provided herein comprises 10 micrograms (μg) or more; 20 micrograms (μg) or more; 30 micrograms (μg) or more; 40 micrograms (μg) or more; 50 micrograms (μg) or more; 1 milligram (mg) or more; 1.5 mg or more; 2.0 mg or more; 2.5 mg or more; 3.0 mg or more; 3.5 mg or more; 4.0 mg or more; 4.5 mg or more; 5 mg or more; 5.5 mg or more; 6.0 mg or more; 6.5 mg or more; 7 mg or more; 7.5 mg or more; 8.0 mg or more; 8.5 mg or more; 9.0 mg or more; 9.5 mg or more; 10 mg or more; 15 mg or more; 20 mg or more; 25 mg or more; 30 mg or more; 35 mg or more; 40 mg or more; 45 mg or more; 50 mg or more; 55 mg or more; 60 mg or more; 65 mg or more; 70 mg or more; 75 mg or more; 80 mg or more; 85 mg or more; 90 mg or more; 95 mg or more; or 100 mg or more; of one or more phospholipids provided herein. For example, for 15 micrograms (μg) of isolated SP-BM, 100 μg DPPC and 50 μg PG can be added (total of 150 μg total phospholipids) to the admixture.

The structure and function of the prepared lung surfactant compositions provided herein (e.g., SP-BN and/or SP-BM) can be assessed by a number of methods known in the art. For example, by lamellar body enrichment, immunostaining, electron microscopy, circular dichroism (CD) spectroscopy, administration of the SP-BM and/or SP-BN to an animal model and monitoring the animal model for a modulation in respiratory function, mass spectrometry, X-ray crystallography, etc. As provided herein in the working examples, reconstitution experiments, CD spectroscopy, and electron microscopy images show that the presence of concentric membrane stacks are a marker for the formation of lamellar bodies and surfactant function.

As discussed above, conservative substitutions in the amino acid sequence or nucleic acid sequence encoding the polypeptides provided herein that permit the formation of the lamellar body structures can be used. For example, directed evolution can be used to subject the polypeptides described herein to random mutagenesis and the resulting polypeptides are screened for desired qualities (e.g, using circular dichroism, thin-section EM, or binding assays). These methods are known in the art. See e.g., Wang et al. Cell, Volume 160, Issue 4, 2015, Pages 785-797; or Daugherty et al. Protein Engineering, Design and Selection, Volume 11, Issue 9, 1998, Pages 825-832.

### Pharmaceutically-Acceptable Carriers and Compositions

The lung surfactant compositions and polypeptides provided herein can be formulated for use in the treatment of a respiratory disease or disorder. In some embodiments of any of the aspects, the composition or nucleic acids encoding such compositions and polypeptides provided herein are formulated with a pharmaceutically acceptable carrier. In some embodiments of any of the aspects, the formulation is administered to a subject with a respiratory disease.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (S) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyllaurate; (13) agar; (14) buffering agents, such as magnesium hydroxide and aluminum hydroxide; (15) alginic acid; (16) pyrogen-free water; (17) isotonic saline; (IS) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

In one embodiment of any of the aspects, the lung surfactant composition further comprises a lipid vehicle. Exemplary lipid vehicles include, but are not limited to, liposomes, phospholipids provided herein, micelles, exosomes, lipid emulsions, and lipid-drug complex.

In another embodiment of any of the aspects, the lung surfactant composition provided herein further comprises a second or additional lung surfactant in an admixture or administered in combination. Non-limiting examples of lung surfactants that can be used in admixture with the compositions provided herein include colfosceril palmitate (EXOSURF®); pumactant (Artificial Lung Expanding Compound or ALEC); venticute; lucinactant (SURFAXIN®); beractant (e.g., Alveofact; Survanta; Beraksurf); calfactant (INFASURF®); poractant alfa (CUROSURF®); KL4 surfactant; recombinant SP-C formulations; or any combination thereof. For example, it is contemplated herein that an admixture of the lung surfactant compositions provided herein and the additional lung surfactant would increase the levels of SP-BM, SP-BN, and SP-BC in the lungs when administered to a subject with ARDS.

In another embodiment of any of the aspects, the lung surfactant composition further comprises a particle or polymer-based vehicle. Exemplary particle or polymer-based vehicles include, but are not limited to, nanoparticles, microparticles, polymer microspheres, or polymer-drug conjugates.

For use as aerosols, the compositions provided herein can be prepared in a solution or suspension and may be packaged in a pressurized aerosol container together with suitable propellants, for example, hydrocarbon propellants like propane, butane, or isobutane with conventional adjuvants.

The lung surfactant compositions provided herein can also be administered in a non-pressurized form such as in a nebulizer or atomizer. The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST™ pneumatic nebulizer available from Inhalation Plastic, Inc. of Niles, Ill. As yet another example, AEROSURF® is a combination of aerosolized KL4 surfactant and an aersol delivery system (ADS) that is less invasive than intubation and mechanical ventilation.

When the active ingredients are adapted to be administered, either together or individually, via nebulizer(s) they can be in the form of a nebulized aqueous suspension or solution, with or without a suitable pH or tonicity adjustment, either as a unit dose or multi-dose device.

Furthermore, any suitable gas can be used to apply pressure during the nebulization, with preferred gases to date being those which are chemically inert. Exemplary gases including, but are not limited to, nitrogen, argon, or helium can be used to high advantage.

In some embodiments, the polypeptides and lung surfactant compositions thereof can also be administered directly to the airways in the form of a dry powder. For use as a dry powder, a SP-BN polypeptide, a SP-BM polypeptide, or lung surfactant compositions thereof can be administered by use of an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The correct dosage of the composition is delivered to the patient.

A dry powder inhaler (i.e., Turbuhaler™ (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of a SP-BN polypeptide, a SP-BM polypeptide, or lung surfactant compositions thereof can be thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), the contents of each of which are incorporated herein by reference in their entirety.

### Respiratory Diseases

In one aspect, provided herein is a method of treating a respiratory disease in a subject, the method comprises: administering the lung surfactant composition provided herein or vector encoding the polypeptides provided herein to a subject in need thereof.

In some embodiments of any of the aspects, the respiratory disease is selected from the group consisting of: acute respiratory distress syndrome (ARDS), neonatal respiratory distress syndrome (NRDS), pneumonia, asthma, meconium aspiration syndrome, respiratory failure, chronic obstructive pulmonary disease (COPD), and a lung infection.

In some embodiments, the respiratory disease is Acute Respiratory Distress Syndrome (ARDS).

ARDS is characterized by shortness of breath, fast breathing, and low oxygen levels in the blood due to abnormal ventilation. Other symptoms of ARDS can include but are not limited to muscle fatigue, weakness, low blood pressure, coughing, blue skin coloration, unresponsiveness, and fever. ARDS can be diagnosed by a skilled practitioner by methods known in the art, e.g., respiratory function tests, chest X rays, CT scan, and blood tests.

By way of example only, the fraction of inspired oxygen (FiO₂) can be used to determine if a subject has ARDS and determine the severity of the disease. The ratio between partial pressure of oxygen in arterial blood (PaO₂) and FiO₂ are used to indicate the level of hypoxemia. A PaO₂/FiO₂ ratio less than or equal to 300 mm Hg indicates a positive diagnosis of ARDS.

ARDS is generally caused by a critical illness. Causes of ARDS include but are not limited to a lung infection, sepsis, trauma, burns, inflammation, drowning or aspiration, drug reactions, or inhalation injuries. In some embodiments, the lung infection is a viral infection (e.g., a coronavirus) or a bacterial infection (e.g., Streptococcus). However, there are some genetic polymorphisms associated with ARDS that are implicated in conferring susceptibility and morbidity in lung injury. For example, see e.g., Reddy and Kleeberger. Pharmacogenomics, (2009) 10(9): 1527-1539, which is incorporated herein by reference in its entirety.

The most common medical interventions for ARDS include intubation, sedation, and ventilation of the subject, along with therapeutic treatments that will improve respiration and reduce inflammation in the lungs. Therapeutics currently used to treat or prevent a respiratory disease include, but are not limited to, antibiotics (e.g., aminosalicylic acid, norflaxacin, penicillin, cephalosporin, azithromycin), antivirals (e.g., zanamivir, oseltamivir), vaccines, corticosteroids (e.g., hydrocortisone, prednisone, prednisolone, budesonide), vasoconstrictors, anti-hypertensive agents, inhalers and bronchodilators (e.g. albuterol, formoterol, salmeterol, tiotropium), anti-inflammatory agents, surfactants (e.g., CUROSURF®), oxygen therapy, ventilation therapy, and any other treatments for respiratory disease are known in the art.

### Administration, Dosing, Efficacy

The lung surfactant compositions provided herein (e.g., SP-BN) can be administered to a subject suspected of having or that has a respiratory disease. A composition comprising the SP-BN polypeptide and/or the SP-BM polypeptide, or lung surfactant compositions thereof can be administered directly to the airways of a subject by aspiration, airway instillation, and/or in the form of an aerosol or by nebulization. The compositions provided herein can be delivered directly to the airway (e.g., trachea) by a skilled practitioner by an endotracheal tube.

In some embodiments of any of the aspects, the methods described herein comprise administering an effective amount of a composition to a subject in order to alleviate at least one symptom of the respiratory disease. In some embodiments, the administering is intratracheal administration.

As used herein, “alleviating at least one symptom of the respiratory disease” is ameliorating any condition or symptom associated with the respiratory disease (e.g., difficulty breathing, too much or too little mucus, coughing, wheezing, gasping for air, inability to breath without a support device, e.g., a ventilator). As compared with an equivalent untreated control, such reduction is by at least 5%, 10%, 20%, 40%, 50%, 60%, 80%, 90%, 95%, 99% or more as measured by any standard technique. A variety of means for administering the agents and compositions described herein to subjects are known to those of skill in the art. In one embodiment of any of the aspects, the agent is administered directly into the trachea or locally (e.g., to the lungs). In one embodiment of any of the aspects, the agent is administered continuously, in intervals, or sporadically. The route of administration of the composition will be optimized for the type of composition being delivered (e.g., a polypeptide or a lung surfactant composition provided herein), and can be determined by a skilled practitioner.

The term “effective amount” as used herein refers to the amount of an agent or composition described herein can be administered to a subject having or diagnosed as having a respiratory disease needed to alleviate at least one or more symptom of the disease. The term “therapeutically effective amount” therefore refers to an amount of a composition or composition that is sufficient to provide a particular anti-respiratory disease effect when administered to a typical subject. An effective amount as used herein, in various contexts, would also include an amount of a composition sufficient to delay the development of a symptom of the disease, alter the course of a symptom of the disease (e.g., slowing the progression of the respiratory disease), or reverse a symptom of the disease (e.g., correcting or halting symptoms of the respiratory disease). Thus, it is not generally practicable to specify an exact “effective amount”. However, for any given case, an appropriate “effective amount” can be determined by one of ordinary skill in the art using only routine experimentation.

In one embodiment of any of the aspects, the composition or composition is administered continuously (e.g., at constant levels over a period of time). Continuous administration of a composition can be achieved, e.g., by continuous release formulations.

In one embodiment of any of the aspects, the composition is administered in intervals (e.g., at various levels over a given period of time).

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a concentration range that includes the IC50 or EC50 (i.e., the concentration of the composition, which achieves a half-maximal inhibition of symptoms) as determined in cell culture, or in an appropriate animal model. Levels in the lung can be measured, for example, by high performance liquid chromatography of a biological sample (e.g., bronchoalveolar lavage fluid). The effects of any particular dosage can be monitored by a suitable bioassay, e.g., measuring respiratory function, or blood work, among others. The dosage can be determined by a physician and adjusted, as necessary, to suit observed effects of the treatment.

The dosage of the composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further compositions, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

In some embodiments of any of the aspects, the composition described herein is used as a monotherapy.

In another embodiment of any of the aspects, the compositions described herein can be used in combination with other known compositions and therapies for a respiratory disease. In another embodiment, of any of the aspects, the compositions provided herein are used in combination with another lung surfactant individually or the compositions provided herein are in admixture with another lung surfactant provided herein.

Administered “in combination,” as used herein, means that two (or more) different treatments are delivered to the subject during the course of the subject's affliction with the disorder, e.g., the two or more treatments are delivered after the subject has been diagnosed with the disorder (a respiratory disease) and before the disorder has been cured or eliminated or treatment has ceased for other reasons. Non-limiting examples of treatments that can be used in combination with the compositions provided herein include colfosceril palmitate (EXOSURF®); pumactant (Artificial Lung Expanding Compound or ALEC); venticute; lucinactant (SURFAXIN®); beractant (e.g., Alveofact; Survanta; Beraksurf); calfactant (INFASURF®); poractant alfa (CUROSURF®); KL4 surfactant; recombinant SP-C formulations; or any combination thereof.

In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The compositions described herein and the at least one additional therapy can be administered simultaneously, in the same or in separate compositions, or sequentially. For sequential administration, the composition described herein can be administered first, and the additional composition can be administered second, or the order of administration can be reversed. The composition and/or other therapeutic compositions, procedures or modalities can be administered during periods of active disorder, or during a period of remission or less active disease. The composition can be administered before another treatment, concurrently with the treatment, post-treatment, or during remission of the disorder.

When administered in combination, the composition and the additional agent or composition (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of a respiratory disease) is lower (e.g., at least 20%, at least 30%, at least 40%, or at least 50% lower) than the amount or dosage of each agent individually required to achieve the same therapeutic effect.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

In some embodiments, the present application may be defined in any of the following paragraphs:
1. A lung surfactant composition comprising: (a) a polypeptide comprising a surfactant B protein N-terminal domain (SP-BN); and (b) at least one phospholipid.
2. The lung surfactant composition of paragraph 1, wherein the polypeptide further comprises a surfactant B protein middle domain (SP-BM) and/or surfactant B protein C-terminal domain (SP-BC).
3. A lung surfactant composition comprising: (a) a polypeptide comprising a surfactant B protein middle domain (SP-BM); and (b) at least one phospholipid.
4. The lung surfactant composition of paragraph 3, wherein the polypeptide lacks a surfactant B protein N-terminal domain (SP-BN) and/or a surfactant B protein C-terminal domain (SP-BC).
5. The lung surfactant composition any one of paragraphs 1-4, wherein the phospholipid is a glycerophospholipid.
6. The lung surfactant composition of any one of paragraphs 1-5, wherein the phospholipid is selected from the group consisting of: 1,2-dipalmitoyl-sn-glycero-3-phosphocholine (DPPC); phosphatidylglycerol (PG); phosphatidylcholine (PC), phosphatidylethanolamine (PE), phosphatidylserine (PS), and phosphatidylinositol (PI).
7. The lung surfactant composition of any one of paragraphs 1-6, wherein the phospholipid is DPPC, PG, or a combination thereof.
8. The lung surfactant composition of any one of paragraphs 1-7, wherein the SP-BN is human, bovine, or mouse SP-BN.
9. The lung surfactant composition of any one of paragraphs 2-8, wherein the SP-BM is human, bovine, or mouse SP-BM.
10. The lung surfactant composition of any one of paragraphs 1-9, wherein the composition is formulated with a pharmaceutically acceptable carrier.
11. The lung surfactant composition of any one of paragraphs 1-10, wherein the composition is formulated for intratracheal delivery.
12. The lung surfactant composition of any one of paragraphs 1-11, further comprising a Surfactant Protein C (SP-C) or a fragment thereof; and/or an additional lung surfactant.
13. The lung surfactant composition of any one of paragraphs 1-12, wherein the SP-BN, the SP-BM, or the SP-BC comprises one or more amino acid substitutions or mutations.
14. The lung surfactant composition of paragraph 13, wherein the SP-BM comprises a C₄₈A amino acid substitution.
15. The lung surfactant composition of paragraph 13, wherein the SP-BN comprises a K46E, P50C, R51E, Y59A, and/or a H79A amino acid substitution, or any combination thereof.
16. A method of treating a respiratory disease in a subject, the method comprising: administering to a subject in need thereof the lung surfactant composition of any one of paragraphs 1-15.
17. The method of paragraph 16, wherein the administering is intratracheal administration.
18. The method of paragraph 16, wherein the respiratory disease is selected from the group consisting of: acute respiratory distress syndrome (ARDS), neonatal respiratory distress syndrome (NRDS), pneumonia, asthma, meconium aspiration syndrome, respiratory failure, chronic obstructive pulmonary disease (COPD), and a lung infection.
19. The method of paragraph 16, wherein the subject is a mammal.
20. The method of paragraph 16 or 19, wherein the subject is a human.
21. A vector comprising a nucleic acid sequence encoding SP-BM; SP-BN; or SP-BN and SP-BM.
22. A method for a preparing a polypeptide comprising a surfactant B protein middle domain (SP-BM), the method comprising: (a) expressing a fusion protein in a cell, wherein the fusion protein comprises: (i) a peptide tag; (ii) a surfactant B protein N-terminal domain (SP-BN); (iii) a linker; and (iv) a SP-BM domain; (b) cleaving the linker in the expressed protein; and (c) isolating or purifying the polypeptide comprising the SP-BM domain.
23. The method of paragraph 22, wherein the linker comprises a cleavage site.
24. The method of paragraph 23, wherein the cleavage site is a protease cleavage site or a chemical cleavage site.
25. The method of paragraph 23 or paragraph 24, wherein the cleavage site is selected from the group consisting of: a tobacco mosaic virus (TEV) protease cleavage site, a thrombin cleavage site, a factor Xa cleavage site, a 3C protease site, and a specific nickel-assisted cleavage (SNAC)-tag.
26. The method of any one of paragraphs 22-25, wherein said isolating or purification in step (c) comprises chromatography.
27. The method of paragraph 26, wherein said isolating or purification in step (c) comprises size exclusion chromatography (SEC).
28. The method of any one of paragraphs 22-27, wherein the method further comprises solubilizing the expressed protein prior to the cleaving step.
29. The method of paragraph 28, wherein said solubilizing comprises solubilizing the expressed protein in a detergent.
30. The method of paragraph 29, wherein said detergent is sarkosyl.
31. The method of any one of paragraphs 22-30, wherein the method further comprises extracting the cleaved polypeptide after the cleaving step.
32. The method of paragraph 31, wherein said extraction is in chloroform/methanol.
33. The method of any one of paragraphs 22-32, further comprising a step of preisolating/prepurifying the cleaved polypeptide after the cleaving step.
34. The method of paragraph 33, wherein said preisolating/prepurifying step comprises contacting the cleaved polypeptide with a hydrophobic resin.
35. The method of any one of paragraphs 22-34, wherein the method further comprises solubilizing the cleaved polypeptide after the cleaving step and prior to the isolating/purifying step.
36. The method of paragraph 35, wherein said solubilizing is in octylglucoside.
37. The method of any one of paragraphs 22-36, wherein the cell is a bacterial cell, a yeast cell, an insect cell, an amphibian cell, or a mammalian cell.
38. The method of paragraph 37, wherein the cell is an Escherichia coli cell.
39. The method of any one of paragraphs 22-38, wherein the fusion protein lacks a surfactant B protein C-terminal domain (SP-BC).
40. The method of any one of paragraphs 22-39, wherein the fusion protein further comprises a saposin polypeptide between the peptide tag and the SP-BN domain.
41. The method of paragraph 40, wherein said saposin polypeptide comprises a saposin A domain (SapA).
42. The method of any one of paragraphs 22-41, wherein the peptide tag is selected from the group consisting of: a glutathione S-transferase (GST) tag, a FLAG-tag, a Myc-tag, HALO, a maltose-binding peptide, and a hexa-his peptide (6×His).
43. The method of any one of paragraphs 22-42, wherein the fusion protein comprises an amino acid sequence of SEQ ID NO: 4 or SEQ ID NO: 16.
44. A polypeptide prepared by a method of any one of paragraphs 22-43.
45. A fusion protein comprising: (i) a peptide tag; (ii) a surfactant B protein N-terminal domain (SP-BN); (iii) a linker; and (iv) a surfactant B protein middle domain (SP-BM) domain.
46. The fusion protein of paragraph 45, where the linker comprises a cleavage site.
47. The fusion protein of paragraph 46, wherein cleavage site is a protease cleavage site.
48. The fusion protein of any one of paragraphs 45-47 wherein the fusion protein lacks a surfactant B protein C-terminal domain (SP-BC).
49. The fusion protein of any one of paragraphs 45-48, wherein the fusion protein further comprises a saposin polypeptide between the peptide tag and the SP-BN domain.
50. The fusion protein of paragraph 49, wherein said saposin polypeptide comprises a saposin A domain (SapA).
51. The fusion protein of any one of paragraphs 41-46, wherein the peptide tag is selected from the group consisting of: a glutathione S-transferase (GST) tag, a FLAG-tag, a Myc-tag, HALO, a maltose-binding peptide, and a hexa-his peptide (6×His).
52. The fusion protein of any one of paragraphs 45-51, wherein the fusion protein comprises an amino acid sequence of SEQ ID NO: 4 or SEQ ID NO: 16.
53. A nucleic acid encoding the fusion protein of any one of paragraphs 45-52.

### EXAMPLES

### Example 1: The Mechanism of Surfactant Protein B (SP-B) Function in Respiration

The methods described herein address the mechanism by which mammalian surfactant protein B (SP-B) functions in respiration. SP-B is essential for breathing in mice and humans. It is made in alveolar type II cells as a precursor containing three domains related to the lysosome-localized saposins. The precursor is proteolytically processed into the individual domains, with the middle domain (SP-BM) currently considered to be the biologically important “mature” protein. SP-BM is initially stored in lamellar bodies (LB), lysosome-like organelles, in which membrane sheets are densely stacked on top of each other. LBs are eventually exocytosed to generate extracellular surfactant, a mixture of phospholipids and protein. The exported lipid bilayers are then transformed into a lipid monolayer at the air-water interface, which reduces surface tension and facilitates breathing. The exact functions of SP-BM and of the other saposin-like domains of SP-B are unknown. Provided herein, biochemistry, structural biology, and cell biology methods can be used to test the hypothesis that the N-terminal and middle domains of SP-B (SP-BN and SP-BM) function together in LB formation. It can be determined whether the C-terminal domain (SP-BC), which is conserved but not essential, augments the function of the other domains. Mice can be tested to determine whether tracheal administration of a mixture of purified SP-BN and SP-BM can replace SP-B made endogenously, and whether the purified proteins have a beneficial effect in mouse models of Acute Respiratory Distress Syndrome (ARDS). These experiments will clarify the structure and function of an essential protein that has remained mysterious for decades, and they will provide the basis for its potential use as a therapeutic.

### Testing the Function of SP-BN and SP-BC:

The present results show that mature SP-BN, which has been largely ignored in the past, is present in vivo as a soluble, secreted protein. Crystal structures and biochemical assays show that recombinantly made SP-BN is a phospholipid transfer protein. SP-BN forms a dimer with a hydrophobic pocket that accommodates three phospholipid molecules. Purified SP-BN can be used to further elucidate the mechanism of lipid transfer. Purified SP-BC will also be tested for lipid transfer activity and for possible augmentation of the activity of SP-BN. To test the biological function of SP-BN, the SP-BN can be introduced to a lipid-transfer-deficient mutant into mice by CRISPR and evaluate respiration and LB formation. In addition, adeno-associated virus (AAV) constructs expressing different SP-B mutants can be used to infect lungs of conditional SP-B knock-out mice and test whether lung function can be maintained by these mutants. Mouse alveolar epithelial cell lines, which faithfully process endogenous SP-B can be used to test the role of SP-BN mutants in LB formation. Bronchoscopy of ARDS patients and control individuals can be performed to test for the presence of SP-BN in lavage fluid.

### Testing the Function of SP-BM:

The present results show that reconstituting purified SP-BM into liposomes results in structures that have a striking resemblance to human LBs when viewed by thin-section electron microscopy (EM). These structures contain a “projection core” of dense protein aggregate from which bubbles of stacked membrane sheets emerge. The mechanism by which SP-BM forms LB-like structures can be addressed using 2D crystallization and biochemical techniques. An expression and purification system for SP-BM is also shown herein, which has been a major bottleneck in the field. The goal is to determine a structure of SP-BM either by X-ray crystallography or cryo-EM. Experiments can show whether SP-BN and SP-BC act synergistically with SP-BM to form LB-like structures in vitro.

Generating a Surfactant with Therapeutic Value:

SP-BN, SP-BM, and SP-BC can be examined separately and together to constitute the active extracellular surfactant. Intratracheal administration of a mixture of purified proteins can be performed to test whether mice expressing SP-B under an inducible promoter retain normal lung function in the absence of inducer in the diet. The results provided herein show that intratracheal administration of purified SP-BN has a beneficial effect in mice treated with lipopolysaccharide (LPS), a model system mimicking ARDS.

Pulmonary surfactant is a mixture of proteins and lipids secreted by alveolar type II cells (reviewed in ¹⁻¹⁰) It forms a thin aqueous layer at the air-water interface and drastically reduces surface tension, thus facilitating breathing. It is generally believed that the water-air interface is formed by a monolayer of phospholipids. The surface area of this monolayer must change during the breathing cycle, probably by continuous conversion of the monolayer into a lipid bilayer and vice versa.

Commercially produced surfactant has been used for decades to treat prematurely born babies who have a delay in generating their own surfactant¹¹. Commercial surfactant, e.g. CUROSURF®, is isolated from bovine or porcine lungs by extraction with organic solvent¹². The mixture contains lipid, mostly dipalmitoyl phosphatidylcholine (DPPC), and 1% (w/w) protein, consisting of surfactant proteins B and C¹³. The surfactant mixture has proven to be ineffective in adults that suffer from Acute Respiratory Distress Syndrome (ARDS)¹⁴. ARDS is a severe condition with high mortality. It can have many different causes, such as sepsis, pancreatitis, trauma, pneumonia, and acid aspiration. The underlying mechanism involves injury to cells that form the air barrier in the lungs, activation of the immune system, and dysfunction of blood clotting. Even though commercial surfactant administration has no effect, surfactant dysfunction may be a major reason for the severity of ARDS^{15,16} Provided herein is a method to identify the molecular function of surfactant protein B (SP-B) and to develop a surfactant formulation that may have therapeutic value in treating ARDS patients.

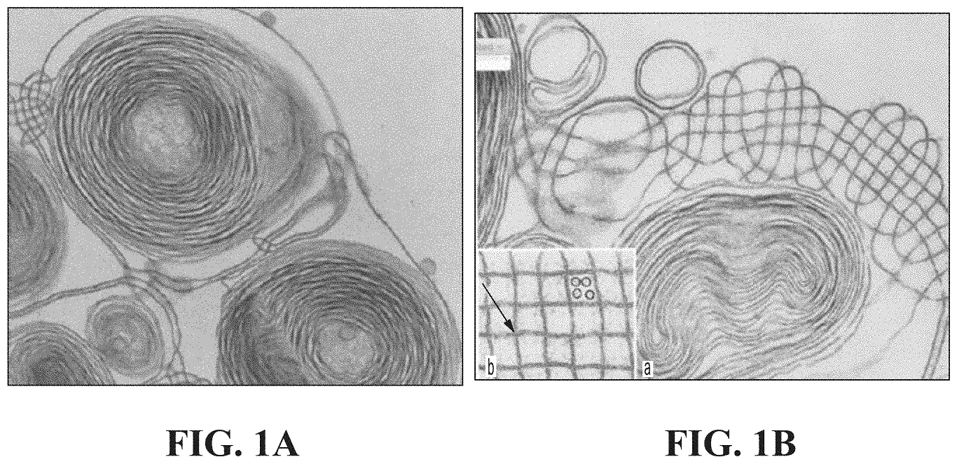

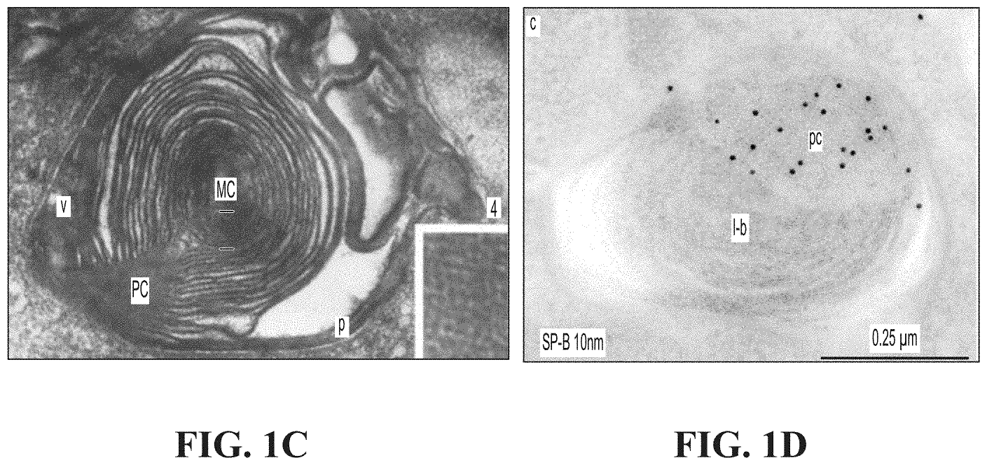

SP-B is the only surfactant protein essential for breathing. It is a conserved protein that is found in all animal species with lungs. SP-B deficiency in mice causes neonatal lethality despite normal embryonic development¹⁷; and mice expressing SP-B under a doxycycline-inducible promoter die within five days after doxycycline withdrawal^{18,19}. In humans, there are rare homozygous cases in which the gene for SP-B is mutated and the protein is not made, resulting in severe respiratory problems that generally require lung transplantation within a few months after birth²⁰⁻²⁸. SP-B is not only involved in reducing the surface tension as a component of the extracellular surfactant, but is also essential for the formation of lamellar bodies (LBs)^{17,29}. LBs are lysosome-like organelles in alveolar type II cells, in which membrane sheets are densely stacked on top of each other like onion layers (FIG. 1A). LBs are the precursor of extracellular surfactant: their limiting membrane fuses with the plasma membrane, ultimately allowing the released internal membrane sheets to be converted into the monolayer at the water-air interface. Once secreted, the membrane sheets are transformed by surfactant protein A into tubular myelin, a unique structure that in cross-section looks like a chess board, consisting of a regular arrangement of membrane squares³⁰⁻³⁴ (FIG. 1B). However, the function of tubular myelin is unclear, as mice lacking surfactant protein A are left with irregular extracellular membrane structures, but breathe normally³⁴.

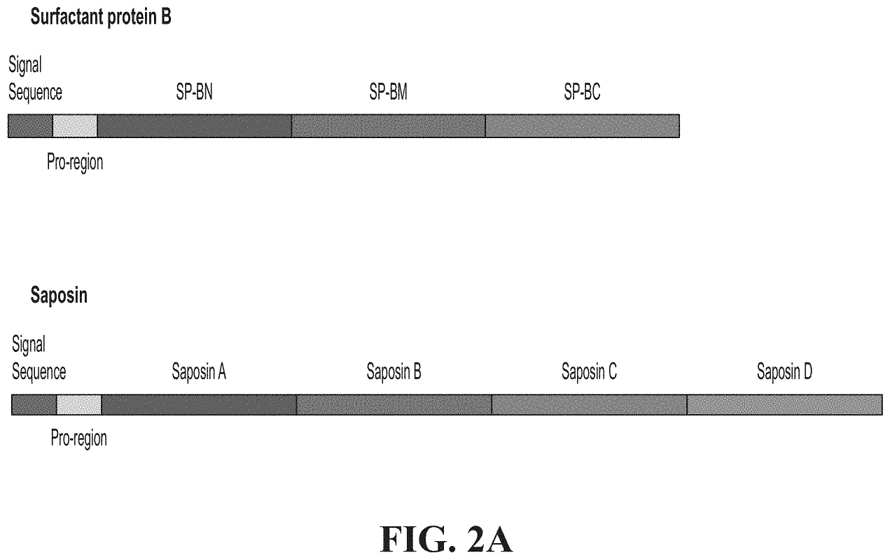

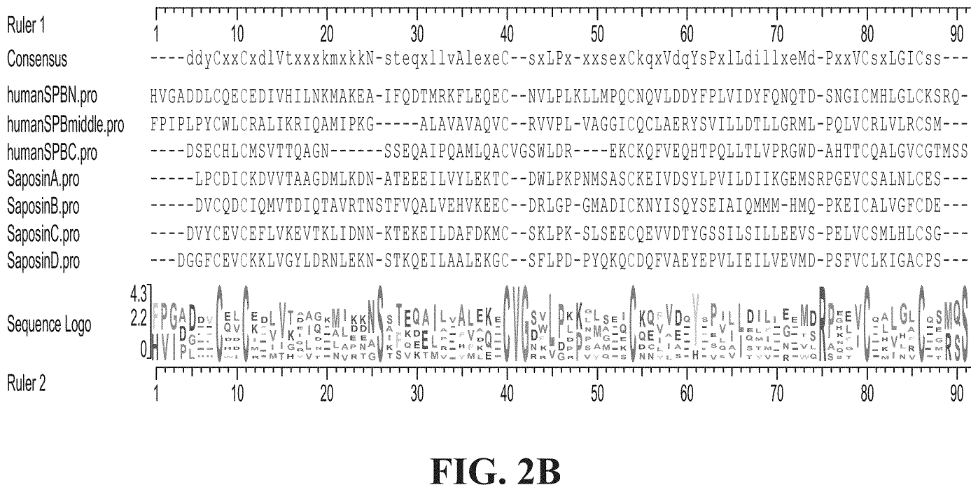

SP-B is made as a precursor, consisting of a signal sequence and three domains that are related to saposins³⁵⁻³⁸ (FIG. 2); the precursor is then proteolytically processed in LBs or precursor organelles into the three individual domains. Saposins are present in lysosomes of all cells and are also made as a precursor, but they contain four, rather than three, related domains (saposins-A, -B, -C, -D; FIG. 2). Saposins are lipid-binding proteins that extract lipids from internal vesicles of multi-vesicular bodies (MVBs), so that they can be degraded by lipases³⁸⁻⁴¹. In the case of SP-B, the middle saposin-like domain (SP-BM) is considered to be the mature protein important for LB formation and surfactant function. SP-BM is therefore often referred to as SP-B, but the term “SP-B” can be used herein only to refer to the full-length precursor protein. The N-terminal saposin-like domain of SP-B (SP-BN) has been reported to have anti-microbial activity⁴² but it has largely been ignored in LB formation and surfactant function. It is not trivial to investigate the function of SP-BN independently of that of SP-BM, as truncations of the gene after the SP-BN-coding region abolish the synthesis of even the N-terminal part of the protein. It therefore remains uncertain whether SP-BN, SP-BM, or both, are essential. The C-terminal saposin-like domain (SP-BC) does not seem to be essential for breathing, as a construct lacking this domain rescues SP-B knock-out mice⁴³.

The mechanism by which LBs are formed and the role of SP-B in this process are poorly understood. In the absence of SP-B, alveolar type II cells contain MVBs but no LBs^{17,29} MVBs are precursors of lysosomes and exist in all cells, but they contain internal vesicles, rather than stacked membrane sheets. MVBs might also be the precursors of LBs, but how the internal vesicles would be converted into sheets is unclear. The phospholipids inside LBs are probably transported into the organelle by an ABC transporter (ABCA3)⁴⁴⁻⁴⁹. This protein is localized to the limiting membrane of LBs, is essential for the formation of LBs, and required for breathing in mice and humans^{45-48,5-55}. The ABC transporter could move phospholipids from the outer to the inner leaflet of the lipid bilayer of the limiting membrane, but how the lipids would subsequently be moved to vesicles or membrane sheets inside the organelle is unknown. Based on this evidence, it can be tested whether this process is mediated by SP-BN serving as a lipid transfer protein.

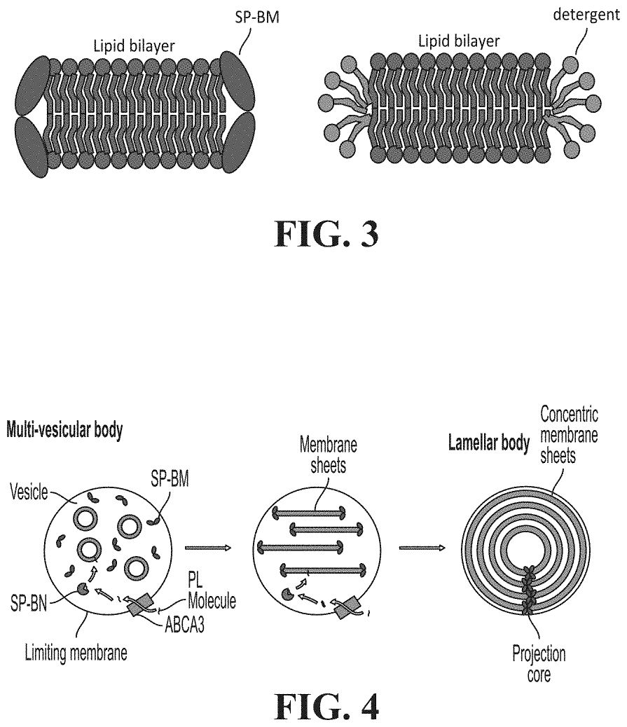

Thin-section electron microscopy (EM) pictures taken 40 years ago show that human LBs have a dense core, called projection core (PC), from which the membrane sheets appear to emerge⁵⁶ (FIG. 1C). This region seems to contain most of the SP-BM molecules, as demonstrated by immuno-EM⁵⁷ (FIG. 1D). The results show that reconstituting purified SP-BM into liposomes results in structures that have a striking resemblance to human LBs (see section 2.1). These structures contain dense protein aggregates, similar to PCs, from which bubbles of stacked membrane sheets emerge. It is postulated that SP-BM alone can form LBs. It would be an amazing result if a protein of only 79 amino acids can form an entire organelle. How exactly SP-BM forms LBs in vitro is unclear, but it is postulated that it localizes to the edges of membrane sheets and stabilizes them, similar to how detergent molecules stabilize bicelles (FIG. 3). In addition, SP-BM seems to self-associate. In this proposal, the mechanism by which SP-BM forms these intriguing structures can be addressed.

Reconstituting an organelle in vitro with purified proteins would be a major achievement. The only other membrane-bound organelle that has been reconstituted with purified proteins is the tubular ER network. In this case, it was possible to reconstitute the organelle with two purified proteins, a curvature-inducing protein of the reticulon or REEP families, and a fusion GTPase (mammalian atlastin or yeast Sey1)⁵⁸.

Even though SP-BM may form LBs in vitro, the process in vivo is likely more complex. It is postulated that both the ABC transporter ABCA3 and SP-BN play a role as well. In the present model (FIG. 4), SP-BN and SP-BM would be present in precursor MVBs that contain internal vesicles. Phospholipids would be flipped across the limiting membrane by ABCA3 and then moved by the lipid-transfer protein SP-BN to internal vesicles. The internal vesicles would then grow as sheets with SP-BM sitting at the edges. Because of the limiting space in the organelle, eventually the sheets have to bend and the only way to add more area to the membrane surface is to decrease the length of the edge. The self-association of SP-BM would anchor all edges in one region, generating the PC (FIG. 4). Next, a multi-faceted approach can be used in which the functions of the individual SP-B domains are studied with purified proteins, using biochemistry and structural biology, the proteins can be followed in intact cells by cell biology techniques, and their roles in vivo can be tested by experiments in mice.

In principle, the endogenously made surfactant can be replaced by a protein-lipid mixture administered exogenously through the trachea. This accomplished with the surfactant replacement therapy for infants. How exactly the surfactant works is unclear, but it is possible that lipids are transferred from the bilayer of the administered liposomes to the monolayer at the lung air surface; the exogenous surfactant would thus boost the generation of the monolayer that has not yet efficiently formed in preterm babies. However, the currently used surfactant mixture is not ideal because it is generated by extraction with organic solvents, it lacks SP-BN, and possibly other hydrophilic proteins, and even the hydrophobic proteins, SP-BM and surfactant protein C (SP-C), are probably present in lower than physiological concentrations. It is possible that the proteins in the currently used surfactants simply cause the lipid bilayer to be disordered, so that lipid molecules are more easily transferred from the bilayer of the liposomes into the monolayer at the lung surface. Such an activity would be expected from any hydrophobic peptide and would not require a specific biological function. Indeed, SP-BM can be replaced by synthetic hydrophobic peptides that correspond to fragments of the protein, which likely cannot fold into the native structure⁵⁹⁻⁶⁹. A non-specific effect might also be exerted by the SP-C component in commercial surfactant. SP-C is made as a single-spanning membrane protein with a short cytosolic N-terminus and a luminal C-terminal segment containing a folded BRICHOS domain^{6,70}. However, only the extremely hydrophobic trans-membrane (TM) segment is present in commercial surfactant, and the rest of the protein, including the BRICHOS domain, is thought to be degraded and irrelevant for the function of the mature protein. The TM segment of SP-C would be expected to non-specifically perturb a lipid bilayer, rather than have a specific function. Given that SP-C is not essential for breathing⁷¹ and that SP-BC may not be essential either⁴³, it is possible that only SP-BN and SP-BM have a defined role in the biological function of surfactant.

There is great interest in generating SP-BM recombinantly to replace the rather old-fashioned source of surfactant from animal sources, but so far, all attempts have failed prior to this disclosure. The reasons for this failure are unclear. As by the methods provided herein, it is now possible to express and purify the other two SP-B domains recombinantly in large amounts (see sections 1.1 and 1.3). Perhaps, the difficulties with SP-BM are caused by its extreme hydrophobicity. One of the goals of this proposal is to develop a recombinant expression system which allows the purification of SP-BM in large amounts. Based on mechanistic insight into the functions of SP-BN and SP-BM and developed expression systems, a surfactant formulation can be generated that is rationally designed, can be generated in large amounts, and may be much more potent than currently used mixtures.

The surface tension-reducing role of surfactant has been known for decades, but the field has been stagnant, leaving the exact function of the surfactant proteins poorly understood. The commercially used surfactant is generated from animal sources in an old-fashioned manner and is likely not optimal. Novel approaches to identify the functions of the domains of surfactant protein B (SP-B), the only essential surfactant protein are provided herein. Purified proteins can be used to determine the structure of the SP-B domains by X-ray crystallography, test their functions by biochemical methods, follow their fate in tissue culture cells by cell biology techniques, and test their role in mice. The combination of these techniques is unique and brings a novel approach to the field. The results indicate that the middle domain of SP-B is sufficient to generate LBs in vitro, which would be an amazing result of high visibility. Based on insight into the mechanistic functions of the SP-B domains, by the methods provided herein, a much better surfactant than is currently available can be generated for therapeutic treatment of ARDS.

### The Function of SP-BN

These experiments are designed to elucidate the function of the N-terminal domain of SP-B (SP-BN), which has been largely ignored in the past. Methods of structural biology, biochemical experiments, and cell biology experiments can be used to test whether SP-BN is a lipid-transfer protein. In vivo experiments in mice will test that whether SP-BN is essential for LB formation. Bronchoscopy of ARDS patients and control individuals can be performed to compare the levels of SP-BN in bronchoalveolar lavage fluid (BALF).

### 1.1. SP-BN is a Lipid-Transfer Protein

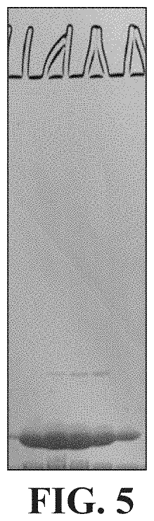

Like all saposin-like proteins, SP-BN contains about 70 to 80 amino acids, including 6 cysteines that form 3 disulfide bridges (FIG. 2). The mouse SP-BN sequence was fused to a His6-tag and thioredoxin, and expressed the fusion protein under an inducible promoter in E. coli cells lacking thioredoxin reductase. This strain allows for the formation of disulfide bridges in the cytosol⁷². The thioredoxin part has also been reported to help with the solubility of the fusion partner⁷³. The fusion protein was purified on a Ni-NTA resin, the thioredoxin part was cleaved off with TEV protease and removed by passage through a second Ni-NTA column. The flow-through was then subjected to gel filtration (FIG. 5). The protein was essentially pure, as judged by SDS-PAGE and Coomassie-blue staining (FIG. 5). The peak fractions were pooled and concentrated to 7-10 mg/ml. Crystallization trials were set up with the hanging drop method at 20° C. Crystals were obtained in 17% PEG 3350, 0.1M citrate buffer pH 5.4, 0.15 M NaCl, cryo-protected with 25% glycerol, and analyzed for diffraction at the 24-ID-C beamline at the APS synchrotron. The crystals diffracted to 2.20 Å resolution (space group 212121). Phasing was done using sulfur single-wavelength anomalous dispersion (SAD). Model building of all four SP-BN dimers in the asymmetric unit was performed with the program COOT. All dimers had essentially the same structure.

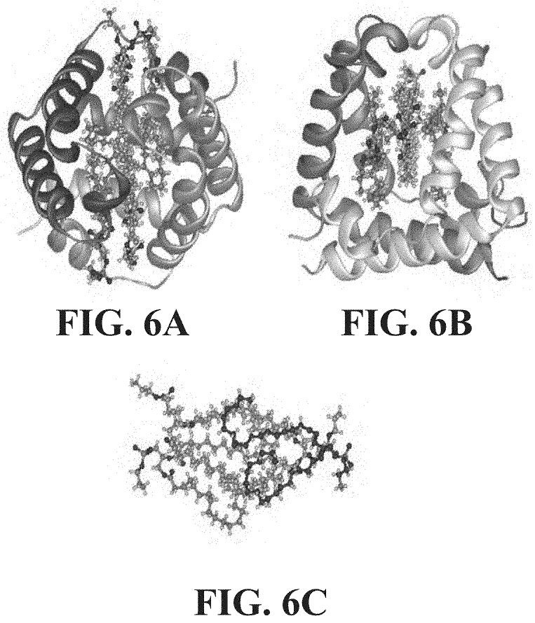

The SP-BN dimer has a hydrophobic pocket between the monomers (FIG. 6). The monomers are associated through two helices at the “floor” and have two loops that form a “roof”. Inside the hydrophobic pocket are three phospholipid (PL) molecules, which must originate from the bacteria in which the protein was expressed. Thin-layer chromatography and mass spectrometry showed that the composition of the SP-BN-associated PL is essentially the same as in E. coli, consisting mostly of phosphatidyl ethanolamine (PE). The PL molecules in the structure contain 16-18 C-atoms, consistent with E. coli PE, but it is uncertain how many double bonds are present in the bound PL molecules. The head groups of the bound PL molecules do not make strong interactions with SP-BN, suggesting that the protein can bind all classes of PLs. Two of the PL molecules are bound at the side of the cavity and have their head groups on opposite ends of the cavity (FIG. 6). Thus, they have the same arrangement as in a lipid bilayer, but the thickness of the “bilayer” is that of a monolayer, as the hydrocarbon chains of both PL molecules curl around (FIG. 6C) and bind into a pocket of SP-BN. The central PL molecule has straight hydrocarbon chains that extend across the entire length of the cavity (FIGS. 6A, C). The occupancy in the crystal structure indicates that the head group of this PL molecule can be on either end of the cavity. Importantly, this PL molecule only makes contact with the hydrocarbon chains of the lateral PL molecules, not with the protein. The central PL molecule thus has the same environment as in a phospholipid mono- or bi-layer. The structure therefore immediately suggests that SP-BN is a lipid transfer protein, which allows the central phospholipid molecule to move with minimal energetic costs between SP-BN and a mono- or bi-layer.

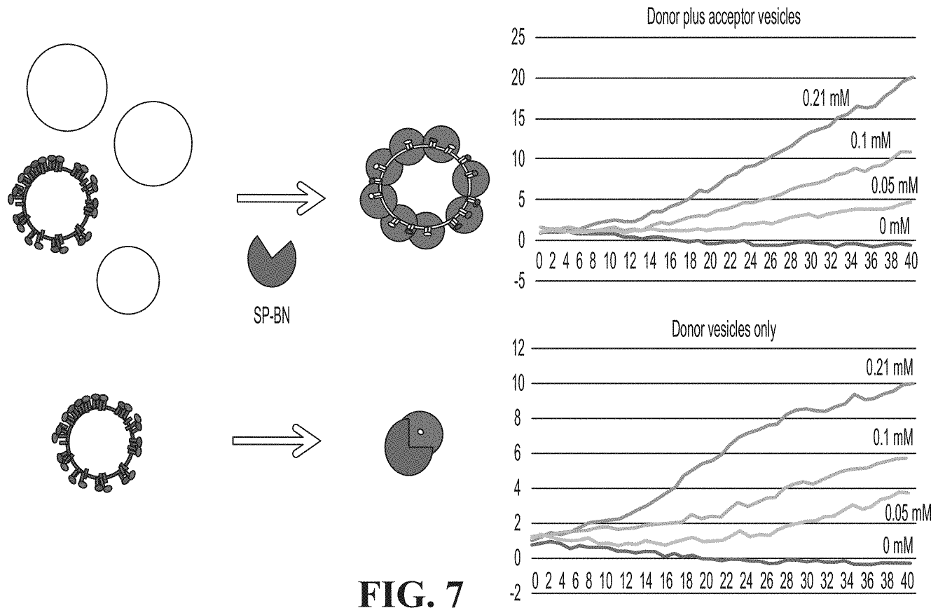

To test whether SP-BN indeed can transfer lipids, fluorescently labeled phospholipids were used in a de-quenching assay (FIG. 7). Emission of the 7-nitrobenzoxadiazole (NBD) fluorescence of labeled PE is initially quenched by rhodamine of the labeled PE, but when the lipids are transferred to unlabeled liposomes or SP-BN molecules, an increase in the emission of NBD is observed. Lipid transfer to another lipid bilayer and to protein molecules (in the absence of unlabeled acceptor vesicles) was observed (FIG. 7). Lipid transfer did not occur at 4° C. and was significantly faster at low pH (pH 5.5) than at neutral pH, consistent with the low pH in LBs⁷⁴.

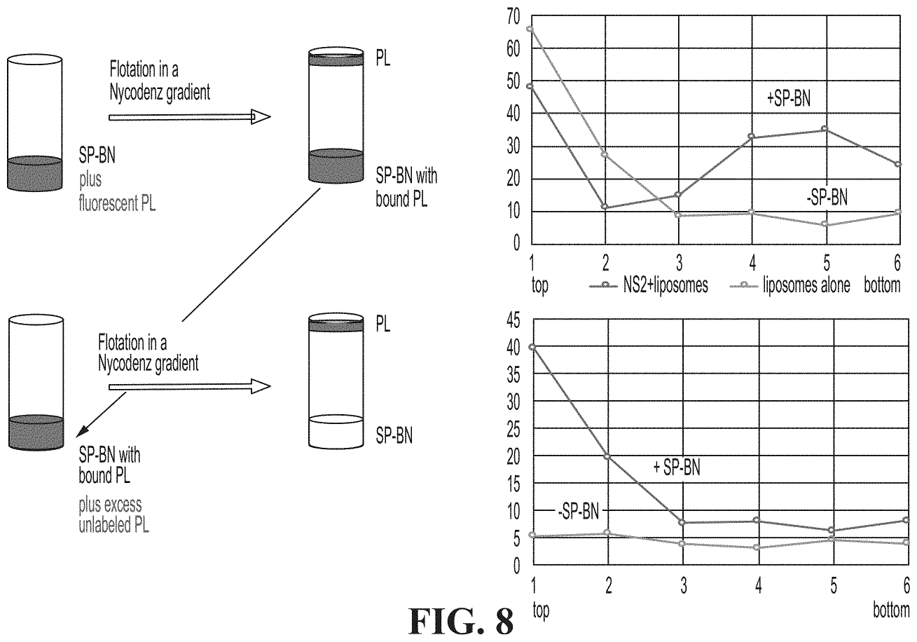

To directly show that SP-BN mediates lipid transport between lipid bilayers, SP-BN was mixed with fluorescently labeled liposomes and floated the vesicles in a Nycodenz gradient (FIG. 8). In the presence of SP-BN, about 50% of the fluorescent lipids were retained at the bottom fractions together with the protein, indicating lipid transfer from the vesicles to the protein. The bottom fraction was then mixed with excess of unlabeled liposomes and subjected to another round of flotation. Now, all the fluorescent lipid floated to the top of the gradient, indicating lipid transfer from SP-BN to the added liposomes.

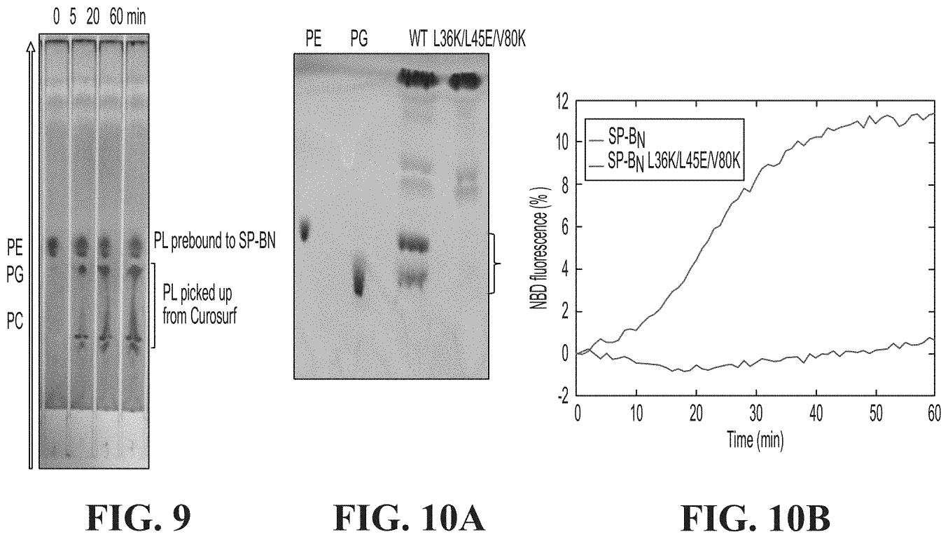

To demonstrate that SP-BN transfers not just fluorescent lipids, a filtration assay was developed. Using centrifugation through 100-kD molecular weight cut-off filters, it was observed that SP-BN moves into the filtrate, while liposomes stay behind. The filtrate can then be subjected to thin-layer chromatography to detect SP-BN-bound lipids. With this assay, it was discovered that phospholipids present in commercial surfactant (CUROSURF®) can be transferred to SP-BN in a time-dependent manner (FIG. 9). Therefore, SP-BN can extract other phospholipids from donor vesicles. Taken together, these experiments indicate that SP-BN is a non-specific lipid-transfer protein.

### 1.2. Elucidating the Mechanism of Lipid Transfer by SP-BN

To no be bound by a particular theory, it is contemplated that the central PL molecule bound to SP-BN can leave the dimeric protein by opening of the “roof”. To test this idea, a cysteine residue at position 50 in the loop forming the “roof”, which should allow disulfide bridge formation between the two monomers and lock the “roof” in a closed state can be used. Lipid transfer should not occur, as measured by the fluorescence de-quenching assay, but should be restored to wild-type levels when the disulfide bridge is reduced by DTT. Although position 50 is most promising, positions 48, 49 and 55 can be used, in case disulfide bridge formation is inefficient even after addition of an oxidant (Cu²⁺-phenanthroline or diamide).

Although SP-BN seems to be a non-specific PL-transfer protein, it is possible that it has a preference for negatively charged lipid bilayers. This assumption is based on the fact that surfactant PL consists largely of neutral DPPC and negatively charged phosphatidyl glycerol (PG) (approximate ratio 7:3), and that there are two positively charged residues pointing outwards from the “roof” (K46 and R51). These residues can be mutated to Ala or Glu and test the purified proteins for bound lipids and for lipid transfer activity. In addition, this can systematically change the lipid composition of donor and acceptor vesicles. The vesicles will contain different percentages of the negatively charged lipids phosphatidyl serine (PS) or PG. By performing the experiments with just donor vesicles (transfer to SP-BN), it can be determined whether there is any charge requirement for the exit of lipids from a bilayer. By keeping the donor composition constant, while varying that of the acceptor vesicles, it can be determined whether charged lipids are necessary for transfer from SP-BN into a bilayer. It will also be identified whether other hydrophobic molecules, such as cholesterol or lipopolysaccharide, can be transferred by SP-BN. These experiments can be performed with the filtration assay.

To further investigate the mechanism of lipid transfer and provide the basis for in vivo tests (see section 1.5), mutants can be generated in SP-BN that are defective in lipid transfer. Several mutants are already made. One mutant, in which the hydrophobicity of the lipid-binding pocket was reduced (I15A, L19A, F28A, F35A, V63A), still contains bound lipid and is only partially defective (about 50% reduction in the de-quenching assay). Curiously, one mutant (Y59A, H79A), removed residues that might interact with the head groups of the bound PL molecules, is more active than the wild-type in the de-quenching assay. A crystal structure of this mutant was determined and found that the four dimers in the asymmetric unit all have different numbers of bound PL molecules, ranging from one to four. In several dimers, the hydrocarbon chains were outside the pocket. These results indicate that Y59 and H79 are important for proper positioning of PL molecules into the binding pocket. After several attempts to generate a mutant that is completely defective in lipid binding, there was success. In this mutant (L36K, L45E, V80K), several hydrophobic residues pointing into the binding pocket are mutated to charged residues. The purified protein behaves well and forms dimers, but it does not contain bound lipids, in contrast to wild-type SP-BN, and it does not transfer lipids (FIG. 10). This mutant can be used for the planned in vivo experiments (section 1.5). A crystal structure of this mutant can be determined, as it would be instructive to visualize SP-BN with an empty binding pocket.

### 1.3. What is the Role of SP-BC?

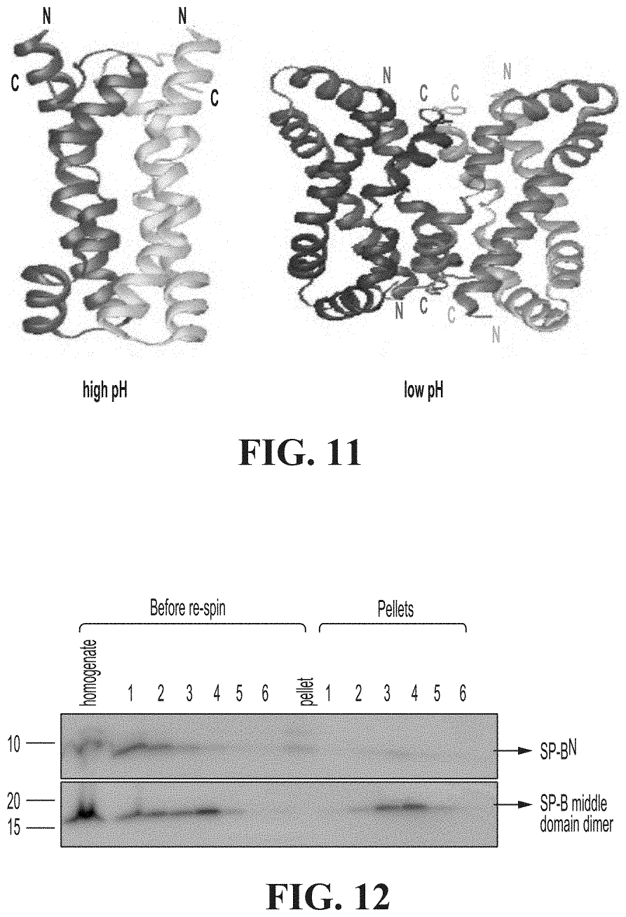

SP-BC does not seem to be essential for breathing, but it is conserved and might have an auxiliary role⁴³. Its sequence is clearly related to the other SP-B domains and to the saposins (FIG. 2). SP-BC was purified the same way as SP-BN, but it does not contain bound lipids, as shown by thin-layer chromatography. The crystal structures of SP-BC in two different conditions were obtained, one at pH 8.5, the other at ˜pH 4.5. The high-pH crystals diffracted to 1.75 Å (space group 212121) and the structure was determined with the program Arcimboldo⁷⁵. The low-pH crystals diffracted to 1.89 Å (space group P1 21 2), and the structure was determined by molecular replacement, using as a search model fragments of the high pH structure. In the high pH structure, SP-BC forms a dimer, in which the two monomers interact through straight helix pairs and are oriented in a parallel manner (FIG. 11). In the low-pH structure, the monomers are anti-parallel and seem to form a tetramer with a small hydrophobic pocket. This structure is probably more meaningful, as LBs have a low pH, as all lysosome-like organelles⁷⁴. Although there is some density in the pocket, it is unclear whether it originates from a lipid or another molecule. As expected from the absence of bound lipid, SP-BC was inactive in lipid transfer assays.

To not be bound by a particular theory, it is contemplated that SP-BC binds an unidentified hydrophobic molecule, like all other saposin-related proteins. It can be tested whether it binds cholesterol, LPS, phosphatidic acid, fatty acids, or gangliosides, using the filter assay in combination with thin-layer chromatography. Another possibility is that SP-BC cooperates with either SP-BN or SP-BM by forming hetero-oligomers. Interaction with SP-BN can be tested both by gel filtration experiments and lipid transfer assays. Cooperation with SP-BM can be tested in reconstitution experiments discussed in section 2.4

### 1.4. SP-BN is a Stable, Soluble Product of SP-B Processing

SP-BN has been largely ignored in the past, mostly because it does not pellet with membranes unlike other surfactant proteins. However, one study reported its presence in lungs, although it was thought to have anti-microbial effects⁴². Given the results that SP-BN is a lipid-transfer protein, its presence in rat lungs was determined. Rat lungs were homogenized and, after a low-speed spin, the supernatant was subjected to sucrose gradient centrifugation. Fractions were separated by SDS-PAGE and immunoblotting with a commercial SP-BM antibody and an antibody that was raised in rabbits, using purified SP-BN as antigen. SP-BM was found in fractions containing LBs (confirmed by EM), whereas SP-BN was found at the top of the gradient (FIG. 12). When the fractions were centrifuged, SP-BN stayed in the supernatant, whereas SP-BM pelleted (FIG. 12). These results indicate that SP-BN is indeed a stable, soluble product of SP-B processing. As expected, SP-BM is membrane-bound.

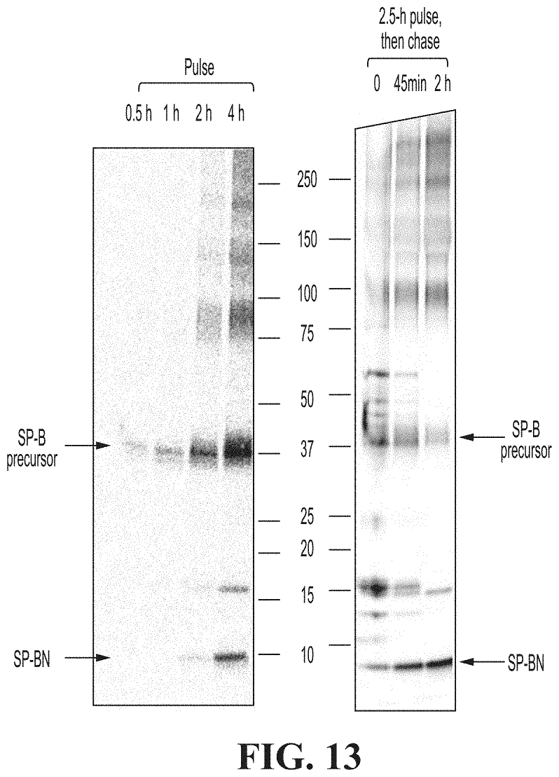

To further test SP-BN synthesis and secretion, alveolospheres generated by directed differentiation of human pluripotent stem cells (PSCs) into type II alveolar cells were used. The alveolospheres have many properties of mature type II cells, such as containing mature SP-BM⁷⁶. Pulse-chase experiments were performed to test whether SP-B is proteolytically processed to SP-BN and SP-BM. The cells were labeled with ³⁵S-methionine for 2.5 hr (pulse), and then for additional time periods with unlabeled methionine (chase). Cell homogenates were subjected to immunoprecipitation with SP-BN or SP-BM antibodies, followed by autoradiography (FIG. 13). The experiments showed that the precursor disappears in favor of SP-BN and SP-BM. SP-BN seems to be as stable as SP-BM.

Based on these experiments, it seems likely that SP-BN is also a stable product of SP-B processing in humans. Thus, with samples obtained by bronchoscopy from normal individuals this can be tested. Given the evidence that SP-BN has a protective effect in a mouse model of ARDS (section 3.2), samples from ARDS patients can also be tested. One outcome of these experiments is that ARDS patients have reduced levels of SP-BN, similar to the decreased levels of SP-BM reported in ARDS patients and in mouse models of ARDS^{15,77}.

1.5. Does SP-BN have a Function In Vivo?

To test the biological function of SP-BN, CRISPR can be used to introduce the mutations L36K, L45E, and V80K (numbering for SP-BN) into both alleles of the SP-B gene of mice. These mutations inactivate the lipid transfer activity of SP-BN (see section 1.2). Introducing point mutations will likely not significantly affect the synthesis and proteolytic processing of SP-B, but this will first be tested in a mouse alveolar epithelial cell line (MLE15) by viral expression of the SP-B mutant (see below). To generate the CRISPR mutant in mice, two guide RNAs can be used, one directed to exon 4, containing L36K, L45E, the other directed to exon 5, containing V80. The Efficient additions can be used with ssDNA inserts-CRISPR (Easi-CRISPR) method with a donor oligo that introduces the mutations and has 50 homologous residues on each side⁷⁸. This will also delete the intron between exons 4 and 5. The CRISPR knock-out can be done by the Mouse Gene Manipulation Core at Children's Hospital Boston. In a previous experiment, in which the coding region following SP-BN was deleted, 6 out of 12 new born mice with the desired mutation in both alleles were obtained. Therefore, it would be expected that a high percentage of mice to carry the mutations in the SP-BN portion of the gene. If heterozygotes are obtained, the mice can be breed to obtain homozygotes. Analysis of the genotype can be performed by PCR and sequencing. If viable off-springs are not obtained, the CRISPR mutation can be repeated again and embryos would be harvested a day before birth. The lungs can be homogenized and proteins can be analyzed by SDS-PAGE and immunoblotting for SP-BM, using a commercial antibody. In addition, Western blot for SP-BN can be used with an antibody. The lungs will also be analyzed for the presence of LBs, using thin-section EM and staining with osmium. If the mutant mice are alive, functional tests can be performed, using a Flexivent instrument. The Inspiratory Capacity (IC), Respiratory System Resistance (Rrs), Compliance (Crs), Elastance (Ers), Newtonian resistance (Rn), Tissue Damping (G), Tissue Elastance (H), and Tissue Hysteresis. Pressure volume (PV) loops can be used to determine Static Compliance (Cst), Inspiratory Capacity (A), Curvature of the upper portion of the PV curve (K), and the Hysteresis area can be measured. Airway and Tissue Elastance, Hysteresis, and particularly Compliance are changed in SP-B conditional knock-out mice at early stages of doxycycline withdrawal from the diet¹⁸, so these will be the most important parameters to judge surfactant dysfunction.

One outcome of these experiments is that mice expressing SP-BN with defective lipid transfer activity are not viable and do not have LBs. This suggests that SP-BN's lipid transfer activity is necessary for LB formation, as hypothesized in the present model (FIG. 4). At the other extreme, the mice might be viable and have no detectable respiratory defects, an outcome that would indicate that SP-BN has no role in LB and surfactant formation. Of course, the results can be in between these extremes. For example, the mice might have LBs, but respiratory defects. Such a result would point to a role of SP-BN at a stage after LB formation, e.g. after their fusion with the plasma membrane.

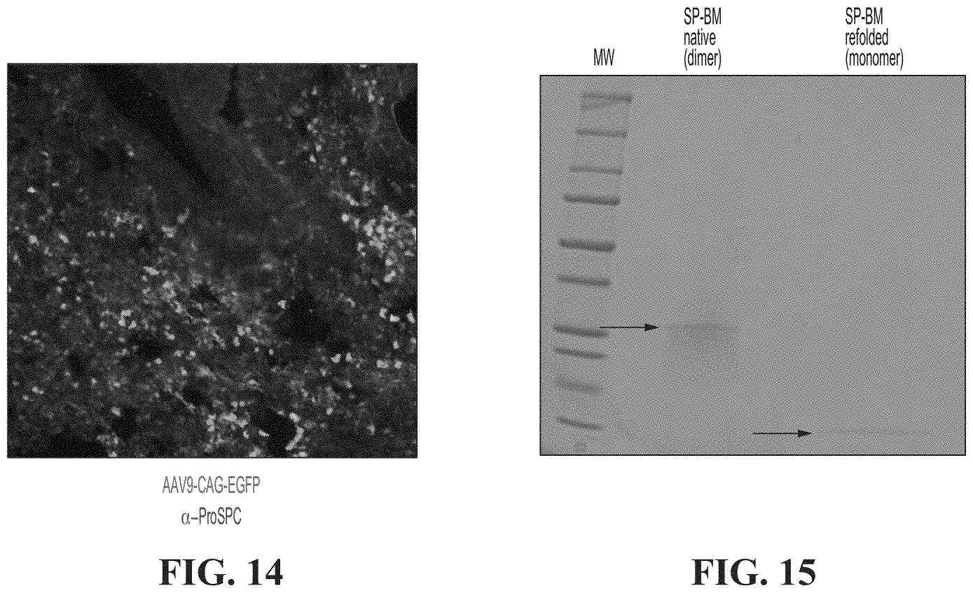

In separate experiments adeno-associated virus (AAV) constructs can be used to infect lungs of conditional SP-B knock-out mice. These mice express full-length SP-B under a doxycycline-inducible promoter and die within 5 days of doxycycline withdrawal¹⁸. There are now about 100 offspring and it has been confirmed that they show obvious signs of respiratory distress after a few days of doxycycline withdrawal from the diet. The AAV constructs can be administered expressing different SP-B mutants through the trachea of these mice. The experiments with wild-type mice show that AAV9 is suitable for these experiments. After 3 weeks of infection with AAV9 expressing EGFP, green fluorescence was seen throughout the entire lung (FIG. 14). The SP-B constructs can be expressed under the strong CAG promoter. Provided that the wild-type SP-B protein rescues mice after withdrawal of doxycycline from the diet, the mutants that contain lipid-transfer defective SP-BN (L36K, L45E, and V80K), lack SP-BC or SP-BM, or contain point mutations in SP-BM (once a structure of SP-BM is confirmed; see section 2.3) can be tested. The mice are tested in a Flexivent instrument, can be observed for signs of respiratory distress at least twice daily, and sacrificed on impending respiratory failure.

To test whether SP-BN has a role in LB formation, a mouse alveolar epithelial cell line (MILE15) can be used. These cells contain LBs and faithfully process endogenous SP-B to SP-BM⁷⁹. The same CRISPR constructs mentioned above can be used, delivered by transfection, to generate a homozygous line that expresses lipid-transfer-defective SP-BN. These cells can be tested for the presence of LBs by thin-section EM. A general SP-B knock-out cell line can be generated and the lentivirus constructs can be used to express various SP-B mutants. Lentivirus expression has been used in other projects⁸⁰. The MILE15 cells will also be used to test for the processing of full-length mutant SP-B into the individual domains. These experiments can be done in the original cell line as human SP-BM has a different size than mouse SP-BM.

### The Function of SP-BM

These experiments are designed to elucidate the function of the middle domain of SP-B (SP-BM). Based on the present results, SP-BM forms LBs in vitro. An expression and purification system for SP-BM has been established, which will provide the basis for elucidating the structure and function of SP-BM.

### 2.1. Reconstituted Purified SP-BM Forms LB-Like Structures In Vitro

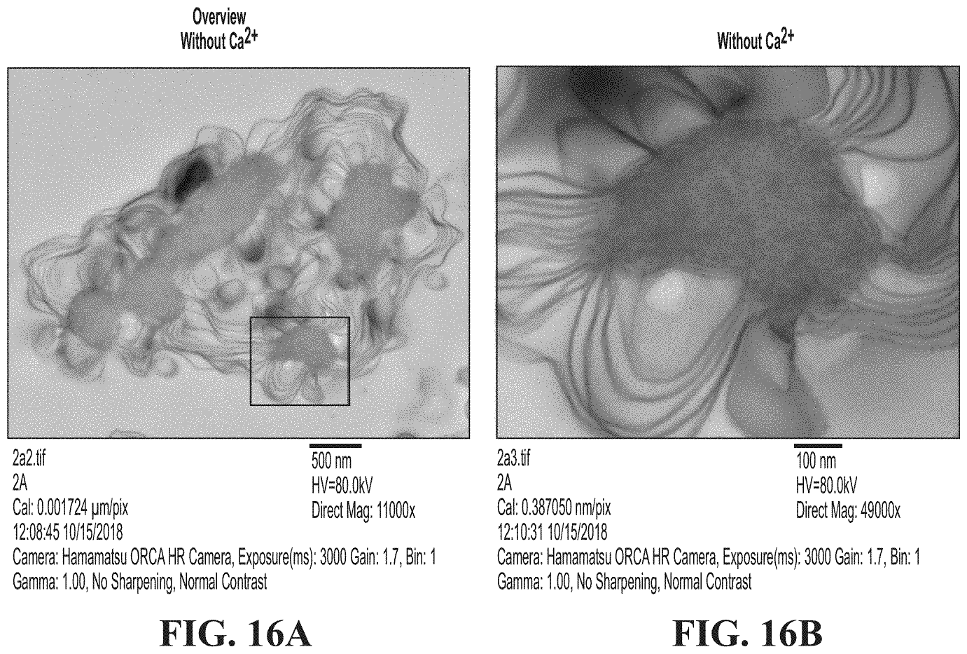

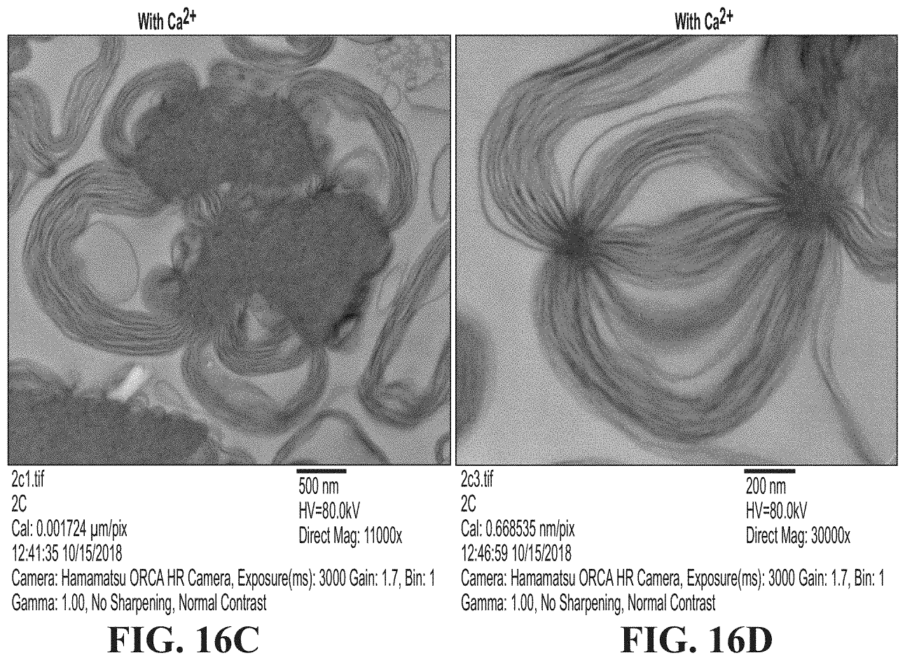

A variation of an established method to purify SP-BM from bovine lungs was developed. Specifically, a lung obtained from the company RESEARCH 87™ was homogenized in a meat grinder and then in a hand-held homogenizer in chloroform/methanol (Bligh and Dyer extraction). The organic phase was collected after phase separation and passed through a 1-m LH20 gel filtration column to separate protein from lipids. The protein fractions were dried and dissolved in a buffer containing 6% octylglucoside (OG). This step is different from previous purification protocols and is based on characterization of SP-BM from rat lungs, which indicated that the protein behaves well in OG. The OG-solubilized material was then subjected to gel filtration on a Superdex 200 column, resulting in fractions that contain essentially only SP-BM (FIG. 15). To reconstitute SP-BM into liposomes, DPPC/PG (2:1) was dried and then added to SP-BM at 10% (w/w) in 1.5% OG. The mixture was diluted 1:5 in buffer without detergent and dialyzed against buffer for 1-5 days. The sample was then subjected to centrifugation and the pellet used for thin-section EM. Strikingly, the images show structures that resemble human LBs (compare FIG. 16 with FIG. 1C). As observed in vivo, multiple stacked membrane bubbles are connected to a protein-rich area that resembles a projection core (PC). As expected, the protein-rich areas become more abundant at higher protein: lipid ratios (not shown). It was also determined whether the addition of C^{a} ions after dialysis would affect the structures observed in vitro. Indeed, the membrane sheets became more stacked, now resembling even more the in vivo situation. Thus, it appears that the SP-BM protein alone, despite its small size, can form an organelle, a stunning result.

### 2.2. Establishment of an Expression and Purification Protocol for SP-BM

Many labs, have tried unsuccessfully to recombinantly express SP-BM; the present protocols for SP-BN and SP-BC could not be applied to SP-BM. The SP-BM was also expressed as thioredoxin fusions, but also fusions to SUMO and maltose-binding protein, expression of SP-BM in the periplasm of E. coli, and expression together with SP-BN or with SP-BN and SP-BC in bacteria, Pichia pastoris, or mammalian cells. The protein was either not expressed or expressed at very low levels. The problem likely originates from the fact that SP-BM is extremely hydrophobic. However, two possible approaches to purify SP-BM recombinantly are provided herein. In the first approach, a fusion protein containing GST, SP-BN, and SP-BM is expressed in E. coli. The protein forms inclusion bodies but is expressed at high levels. The fusion protein can then be solubilized in sarkosyl and the detergent can be replaced by Triton X-100 and CHAPS, which allows refolding of GST and purification on a GSH resin⁸¹. A thrombin-site introduced between SP-BN and SP-BM allows subsequent purification of the cleaved-off SP-BM. In the second approach, full-length SP-B is expressed as a fusion with N-terminal Halo and FLAG tags in mammalian HEK293 cells. The protein is secreted in a soluble form into the medium (about 1 μg/ml) and can be purified with either tag. Elution from the resin can be performed by either cleavage of SP-B, using specific cleavage sites between SP-BN and SP-BM, as well as between SP-BM and SP-BC, or by eluting with FLAG peptide.

Both approaches need further optimization. With the E. coli expression system, it can be tested whether SP-BM is in its native state, as it is possible that the disulfide bridges are incorrectly formed. SP-BM can be purified from native source to develop a protocol for its renaturation after unfolding in urea in the presence of high concentrations of DTT (FIG. 15). This protocol employs dialysis against a redox buffer (GSH/GSSG). This procedure can be used to refold recombinantly made SP-BM. Folding will initially be assessed by solubility of SP-BM and then by the formation of LB-like structures in reconstitution experiments.

2.3. What is the Mechanism by which SP-BM Forms LB-Like Structures In Vitro?

A major goal of this proposal is to determine a structure of SP-BM, which would likely give insight into the mechanism by which it forms LB-like structures. Because the related saposins, SP-BN, and SP-BC all have rather different structures, it is difficult to predict that of SP-BM. Structure determination can be attempted by X-ray crystallography. The first crystallization trials can be made with SP-BM purified from bovine lungs. The methods provided herein describe an optimized purification protocol and can obtain about 0.5 mg from one lung. This is sufficient for initial crystallization trials, either in detergent (OG) or in lipidic cubic phase (LCP). The purification protocol for recombinant SP-BM, can be used to test many more conditions and also generate mutants. One mutant that can be tested is Cys48Ala, as this Cys forms an inter-molecular disulfide bridge (see FIG. 16) and is not essential for SP-BM function⁸². Removal of this Cys might decrease the conformational heterogeneity of the sample.

Another approach to structure determination is 2D crystallization. The projection cores (PCs) of human LBs seem to have a regular pattern (inset in FIG. 1C), suggested to be a hexagonal arrangement of protein molecules. The 2D crystal formation can be tested by mixing in detergent SP-BM with DPPC/PG at a 2-3 g/g ratio, which is about 20 times higher than used in the reconstitutions. The detergent will then be removed by dialysis and the sample can be analyzed by negative-stain EM. The membrane areas with sharp edges can be surveyed and then Fourier transforms can be used to test for crystals. The formation of 2D crystals would already be instructive, as it is postulated that such arrays might explain the emergence of stacked membrane bubbles (FIG. 4). However, a structure of SP-BM using EM diffraction data can also be performed.

To not be bound by a particular theory, it is hypothesized that SP-BM localizes to the edges of membrane sheets (FIG. 4). The 2D crystal structure can show the arrangement of protein and lipid molecules. However, biochemical experiments can also be performed. The reconstitution experiments with SP-BM the lipid 16:0-12:0 NBD-PC, which carries the fluorophore NBD at the end of the shorter hydrocarbon chain can also be added. If SP-BM sits at the edges of a bilayer, it is expected that this result is due to fluorescence quenching. Controls can be performed without added protein and with SP-BM added to preformed multi-lamellar liposomes at a low detergent concentration that is insufficient to solubilize the membranes. Once there is structural information, mutants in SP-BM that inactivate the protein for in vitro LB formation can be engineered. These mutants will then also be tested for quenching of NBD-PC.

It can be tested whether SP-BM can disrupt preformed liposomes, as might be expected if it prefers to localize to the edges of lipid bilayers. These experiments can be done with either full-length SP-B precursor or a precursor containing only SP-BN and SP-BM (section 2.2), neither of which require detergent for solubility. SP-BM is likely inactive as long as it is not released from the SP-B precursor. This process, can therefore add the inactive precursors to preformed liposomes in the absence of detergent and then add thrombin to liberate mature SP-BM. If EM analysis shows that the same structures form as in co-reconstitution experiments, this would indicate the SP-BM is capable of disrupting lipid bilayers. The samples in the above-mentioned quenching assay with fluorescent lipids can then be analyzed.

2.4. Do SP-BN or SP-BC Cooperate with SP-BM in LB Formation or Lipid Transfer?

It is possible that SP-BM cooperates with either SP-BN or SP-BC in LB formation in vitro. A reconstitution assay can be used to test whether the addition of purified SP-BN or SP—BC has an effect on the morphology of LB-like structures formed by SP-BM. The proteins can be added either before removal of the detergent or after formation of liposomes. One outcome of these experiments is that the stacking of membrane sheets is altered. If this is observed with SP-BN, the lipid-transfer defective SP-BN mutant (L36K, L45E, V80K) can be used as a control. This can also test the reverse possibility, i.e. that SP-BM stimulates lipid transfer activity of SP-BN or activates SP-BC for lipid transfer. To this end, purified SP-BN or SP-BC can be added to SP-BM-containing vesicles in the de-quenching lipid-transfer assay. Controls can be performed with multi-lamellar liposomes lacking SP-BM.

Generating a Surfactant with Therapeutic Value

These experiments are designed to generate a surfactant mixture that is superior to existing ones and eventually might be used to treat ARDS patients. The formulation of the surfactant mixture is based on mechanistic insights into the functions of SP-BN and SP-BM as lipid-transfer and LB-forming proteins, respectively (aims #1 and #2). The underlying assumption is that extracellular surfactant that is normally secreted by alveolar type II cells can be replaced by administering an appropriate lipid/protein mixture through the trachea. This mixture will differ from hitherto used surfactant mixtures in that the SP-B proteins can be present at physiological levels and have actual activities, rather than non-specifically disturbing a lipid bilayer. It can be tested whether intratracheal administration of purified SP-B domains has a beneficial effect in mice model systems mimicking ARDS.

### 3.1. Can Endogenous Surfactant be Replaced by an Exogenously Supplied Mixture of Liposomes and Purified SP-B Proteins?

Mice expressing SP-BM under a doxycycline-inducible promoter can be engineered and repeatedly administered a surfactant mixture, starting on the day of doxycycline withdrawal from the diet. Initial experiments can be performed with a mixture that contains liposomes reconstituted with purified SP-BM as well as purified SP-BN. Specifically, proteoliposomes can be used at 50 mg/kg DPPG and PG at a 2:1 ratio, which mimics the composition of LBs, and 5 mg/kg of SP-BM. The concentration of SP-BM is ˜10-fold higher than in the commercial surfactant CUROSURF®⁸³. Proteoliposomes containing the high SP-BM concentration form LB-like structures (section 2.1), whereas proteoliposomes containing a 10-fold lower concentration do not (not shown). SP-BN can be used at 2.5 mg/kg, a dose that caused a beneficial effect in mice treated with LPS (section 3.2). Oral administration of 50 μl of the mixture twice a day under anesthesia can be performed. This frequency of tracheal injection was chosen because surfactant is degraded with a half-life of about 0.5 days⁷, and more frequent anesthesia may cause other distress in the animals. As before the mice can be tested in a Flexivent instrument, observe them for signs of respiratory distress at least twice daily, and sacrifice them on impending respiratory failure.

One outcome of these experiments is that the mice show no signs of respiratory distress even after several days of doxycycline withdrawal. Such a result would indicate that exogenous surfactant is as effective as the one endogenously made. In this case, further experiments can be performed to test whether both SP-BM and SP-BN are necessary to see a beneficial effect. In addition, aerosols for administration of the surfactant can be used and tested for whether the protein concentration can be reduced.

Of course, it is possible that the mice still undergo respiratory distress after administration of the surfactant mixture. Such a result may not necessarily indicate that the surfactant composition is inappropriate; rather, it could also be caused by ineffective administration of the mixture or its degradation. If partial beneficial effects are observed, the frequency and method of administration can be varied, as well as the protein concentrations. Purified SP-BC can further be added, as it might enhance the efficiency of the mixture.

### 3.2. Testing Surfactant Mixtures in Mouse Models of ARDS

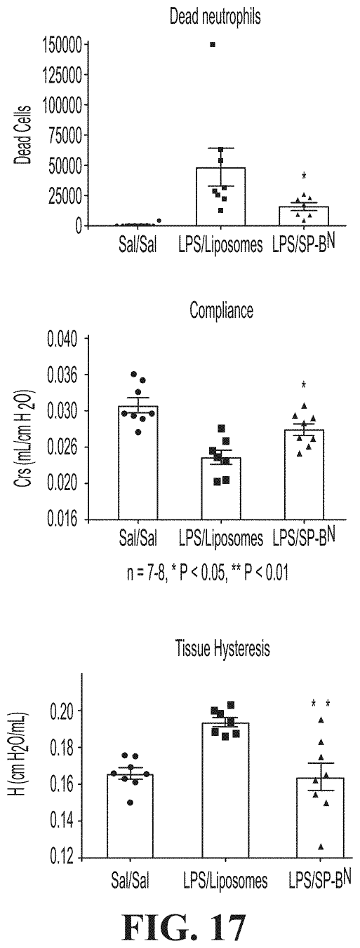

Wild-type C₅₇BL/6 mice will be administered lipopolysaccharide (LPS) through the trachea to cause lung injury. This is an established model for ARDS⁸⁴. It was then tested whether subsequent intratracheal administration of SP-BN has a beneficial effect. Specifically, 0.4 mg/kg LPS was injected, and then administered twice (after 1 and 21 hours) either liposomes alone (50 mg/kg) or liposomes containing SP-BN (2.5 mg/kg). At 24 hours, the mice were tested with a Flexivent and then sacrificed. A number of immunological parameters were determined, such as inflammatory cells in BALF, efferocytosis of neutrophils by macrophages, and the number of neutrophils. The present data show that SP-BN has indeed a beneficial effect (FIG. 17) (3 experiments with a total of 8 mice tested per condition). An improvement in the Compliance and Tissue Hysteresis, parameters that would be expected to reflect surfactant function was observed. A decrease in dead neutrophils was observed (FIG. 17), likely reflecting reduced inflammation, consistent with conditional SP-B knock-out mice showing increased inflammation when doxycycline is removed from the diet¹⁹.

The surfactant mixtures used in section 3.1 will also be used in mouse models of ARDS. Given that overexpressing full-length SP-B in transgenic mice inhibits LPS-induced inflammation⁸⁵ and SP-BN already had a beneficial effect (FIG. 17), it is contemplated that a mixture that contains additionally reconstituted SP-BM will be even more effective. If enhanced efficiency is observed, SP-BM-containing liposomes can be tested in the absence of SP-BN. As controls, saline can be injected, in addition to liposomes together with a lipid-transfer-defective SP-BN mutant, and mutants in SP-BM defective in LB formation in vitro. Although it is generally thought that LPS injury in mice is a reasonable model for ARDS, other models have been used. As another alternative, lung injury by acid can be utilized⁸⁶. Specifically, 0.1 N HCl can be instilled directly into trachea, which gives rise to an acute inflammatory response, increase in airway resistance and a decrease in lung compliance. Obviously, these experiments are limited by the number of mice that can reasonably be tested simultaneously. As in the past, 3 mice for each condition can be used and the experiments can be repeated to obtain statistically significant data.

### REFERENCES

1. Lopez-Rodriguez, E., Gay-Jordi, G., Mucci, A., Lachmann, N. & Serrano-Mollar, A. Lung surfactant metabolism: early in life, early in disease and target in cell therapy. Cell Tissue Res. 367, 721-735 (2017).
2. Pérez-Gil, J. Structure of pulmonary surfactant membranes and films: the role of proteins and lipid-protein interactions. Biochim. Biophys. Acta 1778, 1676-1695 (2008).
3. Bernhard, W. Lung surfactant: Function and composition in the context of development and respiratory physiology. Ann. Anat. 208, 146-150 (2016).
4. Creuwels, L. A., van Golde, L. M. & Haagsman, H. P. The pulmonary surfactant system: biochemical and clinical aspects. Lung 175, 1-39 (1997).
5. Johansson, J. & Curstedt, T. Molecular structures and interactions of pulmonary surfactant components. Eur. J. Biochem. 244, 675-693 (1997).
6. Johansson, J., Curstedt, T. & Robertson, B. The proteins of the surfactant system. Eur. Respir. J. 7, 372-391 (1994).
7. Ikegami, M. & Jobe, A. H. Surfactant protein metabolism in vivo. Biochim. Biophys. Acta 1408, 218-225 (1998).
8. Floros, J., Phelps, D., Pison, U. & Spragg, R. Pulmonary Surfactant-Update on Function, Molecular Biology and Clinical Implications. Curr Respir Med Rev 1, 77-84 (2005).
9. Rooney, S. a, Young, S. L. & Mendelson, C. R. Molecular and cellular processing of lung surfactant. FASEB journal: official publication of the Federation of American Societies for Experimental Biology 8, 957-967 (1994).
10. Benatar, S. R. Pulmonary surfactant. S. Afr. Med. J. 56, 161-162 (1979).
11. Poulain, F. R. & Clements, J. A. Pulmonary surfactant therapy. West J Med 162, 43-50 (1995).
12. Wiseman, L. R. & Bryson, H. M. Porcine-derived lung surfactant. A review of the therapeutic efficacy and clinical tolerability of a natural surfactant preparation (Curosurf) in neonatal respiratory distress syndrome. Drugs 48, 386-403 (1994).
13. Mathialagan, N. & Possmayer, F. Low-molecular-weight hydrophobic proteins from bovine pulmonary surfactant. Biochim. Biophys. Acta 1045, 121-127 (1990).
14. Meng, S.-S. et al. Effect of surfactant administration on outcomes of adult patients in acute respiratory distress syndrome: a meta-analysis of randomized controlled trials. BMC Pulm Med 19, 9 (2019).
15. Gregory, T. J. et al. Surfactant chemical composition and biophysical activity in acute respiratory distress syndrome. J. Clin. Invest. 88, 1976-1981 (1991).
16. Petty, T. L., Silvers, G. W., Paul, G. W. & Stanford, R. E. Abnormalities in lung elastic properties and surfactant function in adult respiratory distress syndrome. Chest 75, 571-574 (1979).
17. Clark, J. C. et al. Targeted disruption of the surfactant protein B gene disrupts surfactant homeostasis, causing respiratory failure in newborn mice. Proc. Natl. Acad. Sci. USA 92, 7794-7798 (1995).
18. Melton, K. R. et al. SP-B deficiency causes respiratory failure in adult mice. Am. J. Physiol. Lung Cell Mol. Physiol. 285, L543-9 (2003).
19. Ikegami, M., Whitsett, J. A., Martis, P. C. & Weaver, T. E. Reversibility of lung inflammation caused by SP-B deficiency. Am. J. Physiol. Lung Cell Mol. Physiol. 289, L962-70 (2005).
20. Nogee, L. M. et al. A mutation in the surfactant protein B gene responsible for fatal neonatal respiratory disease in multiple kindreds. J. Clin. Invest. 93, 1860-1863 (1994).
21. Nogee, L. M., de Mello, D. E., Dehner, L. P. & Colten, H. R. Brief report: deficiency of pulmonary surfactant protein B in congenital alveolar proteinosis. N. Engl. J. Med. 328, 406-410 (1993).
22. Tredano, M. et al. Compound SFTPB 1549C-->GAA (121ins2) and 457delC heterozygosity in severe congenital lung disease and surfactant protein B (SP-B) deficiency. Hum. Mutat. 14, 502-509 (1999).
23. Kurath-Koller, S., Resch, B., Kraschl, R., Windpassinger, C. & Eber, E. Surfactant Protein B Deficiency Caused by Homozygous C248X Mutation-A Case Report and Review of the Literature. AJP Rep. 5, e53-9 (2015).
24. Wallot, M. et al. Congenital alveolar proteinosis caused by a novel mutation of the surfactant protein B gene and misalignment of lung vessels in consanguineous kindred infants. Eur. J. Pediatr. 158, 513-518 (1999).
25. Schuerman, F. A. B. A. et al. Surfactant protein B deficiency caused by a novel mutation involving multiple exons of the SP-B gene. Eur J Med Res 13, 281-286 (2008).
26. Nogee, L. M., Wert, S. E., Proffit, S. A., Hull, W. M. & Whitsett, J. A. Allelic heterogeneity in hereditary surfactant protein B (SP-B) deficiency. Am. J. Respir. Crit. Care Med. 161, 973-981 (2000).
27. Lin, Z., deMello, D. E., Wallot, M. & Floros, J. An SP-B gene mutation responsible for SP-B deficiency in fatal congenital alveolar proteinosis: evidence for a mutation hotspot in exon 4. Mol. Genet. Metab. 64, 25-35 (1998).
28. Wegner, D. J. et al. A major deletion in the surfactant protein-B gene causing lethal respiratory distress. Acta Paediatr. 96, 516-520 (2007).
29. Stahlman, M. T., Gray, M. P., Falconieri, M. W., Whitsett, J. A. & Weaver, T. E. Lamellar body formation in normal and surfactant protein B-deficient fetal mice. Lab. Invest. 80, 395-403 (2000).
30. Poulain, F. R., Allen, L., Williams, M. C., Hamilton, R. L. & Hawgood, S. Effects of surfactant apolipoproteins on liposome structure: implications for tubular myelin formation. Am. J. Physiol. 262, L730-9 (1992).
31. Williams, M. C., Hawgood, S. & Hamilton, R. L. Changes in lipid structure produced by surfactant proteins SP-A, SP-B, and SP-C. Am. J. Respir. Cell Mol. Biol. 5, 41-50 (1991).
32. Larsson, M., van Iwaarden, J. F., Haitsma, J. J., Lachmann, B. & Wollmer, P. Human SP-A and a pharmacy-grade porcine lung surfactant extract can be reconstituted into tubular myelin—a comparative structural study of alveolar surfactants using cryo-transmission electron microscopy. Clin. Physiol. Funct. Imaging 23, 199-203 (2003).
33. Wang, G., Guo, X., Diangelo, S., Thomas, N. J. & Floros, J. Humanized SFTPA1 and SFTPA2 transgenic mice reveal functional divergence of SP-A1 and SP-A2: formation of tubular myelin in vivo requires both gene products. J. Biol. Chem. 285, 11998-12010 (2010).
34. Korfhagen, T. R. et al. Altered surfactant function and structure in SP-A gene targeted mice. Proc. Natl. Acad. Sci. USA 93, 9594-9599 (1996).
35. Patthy, L. Homology of the precursor of pulmonary surfactant-associated protein SP-B with prosaposin and sulfated glycoprotein 1. J. Biol. Chem. 266, 6035-6037 (1991).
36. Olmeda, B., Garcia-Álvarez, B. & Pérez-Gil, J. Structure-function correlations of pulmonary surfactant protein SP-B and the saposin-like family of proteins. Eur Biophys J 42, 209-222 (2013).
37. Zaltash, S. & Johansson, J. Secondary structure and limited proteolysis give experimental evidence that the precursor of pulmonary surfactant protein B contains three saposin-like domains. FEBS Lett. 423, 1-4 (1998).
38. Bruhn, H. A short guided tour through functional and structural features of saposin-like proteins. Biochem. J. 389, 249-257 (2005).
39. Vaccaro, A. M., Salvioli, R., Tatti, M. & Ciaffoni, F. Saposins and their interaction with lipids. Neurochem. Res. 24, 307-314 (1999).
40. Alattia, J.-R., Shaw, J. E., Yip, C. M. & Privé, G. G. Direct visualization of saposin remodelling of lipid bilayers. J. Mol. Biol. 362, 943-953 (2006).
41. Munford, R. S., Sheppard, P. O. & O'Hara, P. J. Saposin-like proteins (SAPLIP) carry out diverse functions on a common backbone structure. J. Lipid Res. 36, 1653-1663 (1995).
42. Yang, L. et al. Surfactant protein B propeptide contains a saposin-like protein domain with antimicrobial activity at low pH. J. Immunol. 184, 975-983 (2010).
43. Akinbi, H. T. et al. Rescue of SP-B knockout mice with a truncated SP-B proprotein. Function of the C-terminal propeptide. J. Biol. Chem. 272, 9640-9647 (1997).
44. Matsumura, Y., Sakai, H., Sasaki, M., Ban, N. & Inagaki, N. ABCA3-mediated choline-phospholipids uptake into intracellular vesicles in A549 cells. FEBS Lett. 581, 3139-3144 (2007).
45. Hammel, M. et al. Targeted inactivation of the murine Abca3 gene leads to respiratory failure in newborns with defective lamellar bodies. Biochem. Biophys. Res. Commun. 359, 947-951 (2007).
46. Cheong, N. et al. ABCA3 is critical for lamellar body biogenesis in vivo. J. Biol. Chem. 282, 23811-23817 (2007).
47. Ban, N. et al. ABCA3 as a lipid transporter in pulmonary surfactant biogenesis. J. Biol. Chem. 282, 9628-9634 (2007).
48. Fitzgerald, M. L. et al. ABCA3 inactivation in mice causes respiratory failure, loss of pulmonary surfactant, and depletion of lung phosphatidylglycerol. J. Lipid Res. 48, 621-632 (2007).
49. Nagata, K. et al. Human ABCA3, a product of a responsible gene for abca3 for fatal surfactant deficiency in newborns, exhibits unique ATP hydrolysis activity and generates intracellular multilamellar vesicles. Biochem. Biophys. Res. Commun. 324, 262-268 (2004).
50. Peca, D., Cutrera, R., Masotti, A., Boldrini, R. & Danhaive, O. ABCA3, a key player in neonatal respiratory transition and genetic disorders of the surfactant system. Biochem. Soc. Trans. 43, 913-919 (2015).
51. Besnard, V. et al. Conditional deletion of Abca3 in alveolar type II cells alters surfactant homeostasis in newborn and adult mice. Am. J. Physiol. Lung Cell Mol. Physiol. 298, L646-59 (2010).
52. Wert, S. E., Whitsett, J. A. & Nogee, L. M. Genetic disorders of surfactant dysfunction. Pediatr. Dev. Pathol. 12, 253-274 (2009).
53. Shulenin, S. et al. ABCA3 gene mutations in newborns with fatal surfactant deficiency. N. Engl. J. Med. 350, 1296-1303 (2004).
54. Mulugeta, S. et al. Identification of LBM180, a lamellar body limiting membrane protein of alveolar type II cells, as the ABC transporter protein ABCA3. J. Biol. Chem. 277, 22147-22155 (2002).
55. Yamano, G. et al. ABCA3 is a lamellar body membrane protein in human lung alveolar type II cells. FEBS Lett. 508, 221-225 (2001).
56. Stratton, C. J. The ultrastructure of multilamellar bodies and surfactant in the human lung. Cell Tissue Res. 193, 219-229 (1978).
57. Brasch, F. et al. Surfactant protein B in type II pneumocytes and intra-alveolar surfactant forms of human lungs. Am. J. Respir. Cell Mol. Biol. 30, 449-458 (2004).
58. Powers, R. E., Wang, S., Liu, T. Y. & Rapoport, T. A. Reconstitution of the tubular endoplasmic reticulum network with purified components. Nature 543, 257-260 (2017).
59. Johansson, J. & Curstedt, T. Synthetic surfactants with SP-B and SP-C analogues to enable worldwide treatment of neonatal respiratory distress syndrome and other lung diseases. J. Intern. Med. 285, 165-186 (2019).
60. Choi, Y. S., Chung, S. H. & Bae, C. W. A combination of short and simple surfactant protein B and C analogues as a new synthetic surfactant: in vitro and animal experiments. Yonsei Med J 58, 823-828 (2017).
61. Walther, F. J., Gordon, L. M. & Waring, A. J. Design of surfactant protein B peptide mimics based on the saposin fold for synthetic lung surfactants. Biomed. Hub 1, (2016).
62. Sweet, D. G. et al. A first-in-human clinical study of a new SP-B and SP-C enriched synthetic surfactant (CHF5633) in preterm babies with respiratory distress syndrome. Arch. Dis. Child. Fetal Neonatal Ed. 102, F497-F503 (2017).
63. Ricci, F., Murgia, X., Razzetti, R., Pelizzi, N. & Salomone, F. In vitro and in vivo comparison between poractant alfa and the new generation synthetic surfactant CHF5633. Pediatr. Res. 81, 369-375 (2017).
64. Notter, R. H., Wang, Z. & Walther, F. J. Activity and biophysical inhibition resistance of a novel synthetic lung surfactant containing Super-Mini-B DATK peptide. PeerJ 4, e1528 (2016).
65. Sato, A. & Ikegami, M. SP-B and SP-C containing new synthetic surfactant for treatment of extremely immature lamb lung. PLoS One 7, e39392 (2012).
66. Lal, M. K. & Sinha, S. K. Surfactant respiratory therapy using Surfaxin/sinapultide. Ther Adv Respir Dis 2, 339-344 (2008).
67. Bruni, R., Hernández-Juviel, J. M., Tanoviceanu, R. & Walther, F. J. Synthetic mimics of surfactant proteins B and C: in vitro surface activity and effects on lung compliance in two animal models of surfactant deficiency. Mol. Genet. Metab. 63, 116-125 (1998).
68. Revak, S. D. et al. Efficacy of synthetic peptide-containing surfactant in the treatment of respiratory distress syndrome in preterm infant rhesus monkeys. Pediatr. Res. 39, 715-724 (1996).
69. Cochrane, C. G. et al. The efficacy and safety of KL4-surfactant in preterm infants with respiratory distress syndrome. Am. J. Respir. Crit. Care Med. 153, 404-410 (1996).
70. Casals, C. et al. C-terminal, endoplasmic reticulum-lumenal domain of prosurfactant protein C—structural features and membrane interactions. FEBS J. 275, 536-547 (2008).
71. Glasser, S. W. et al. Altered stability of pulmonary surfactant in SP-C-deficient mice. Proc. Natl. Acad. Sci. USA 98, 6366-6371 (2001).
72. Derman, A. I., Prinz, W. A., Belin, D. & Beckwith, J. Mutations that allow disulfide bond formation in the cytoplasm of Escherichia coli. Science 262, 1744-1747 (1993).
73. McCoy, J. & La Ville, E. Expression and purification of thioredoxin fusion proteins. Curr Protoc Protein Sci Chapter 6, Unit 6.7 (2001).
74. Chander, A., Johnson, R. G., Reicherter, J. & Fisher, A. B. Lung lamellar bodies maintain an acidic internal pH. J. Biol. Chem. 261, 6126-6131 (1986).
75. Rodriguez, D. et al. Practical structure solution with ARCIMBOLDO. Acta Crystallogr. Sect. D, Biol. Crystallogr. 68, 336-343 (2012).
76. Jacob, A. et al. Differentiation of Human Pluripotent Stem Cells into Functional Lung Alveolar Epithelial Cells. Cell Stem Cell 21, 472-488.e10 (2017).
77. Ingenito, E. P. et al. Decreased surfactant protein-B expression and surfactant dysfunction in a murine model of acute lung injury. Am. J. Respir. Cell Mol. Biol. 25, 35-44 (2001).
78. Quadros, R. M. et al. Easi-CRISPR. a robust method for one-step generation of mice carrying conditional and insertion alleles using long ssDNA donors and CRISPR ribonucleoproteins. Genome Biol. 18, 92 (2017).
79. Wikenheiser, K. A. et al. Production of immortalized distal respiratory epithelial cell lines from surfactant protein C/simian virus 40 large tumor antigen transgenic mice. Proc. Natl. Acad. Sci. USA 90, 11029-11033 (1993).
80. Wang, S., Tukachinsky, H., Romano, F. B. & Rapoport, T. A. Cooperation of the ER-shaping proteins atlastin, lunapark, and reticulons to generate a tubular membrane network. Elife 5, (2016).
81. Tao, H. et al. Purifying natively folded proteins from inclusion bodies using sarkosyl, Triton X-100, and CHAPS. BioTechniques 48, 61-64 (2010).
82. Beck, D. C. et al. The role of homodimers in surfactant protein B function in vivo. J. Biol. Chem. 275, 3365-3370 (2000).
83. Bernhard, W. et al. Commercial versus native surfactants. Surface activity, molecular components, and the effect of calcium. Am. J. Respir. Crit. Care Med. 162, 1524-1533 (2000).
84. D'Alessio, F. R. Mouse models of acute lung injury and ARDS. Methods Mol. Biol. 1809, 341-350 (2018).
85. Epaud, R. et al. Surfactant protein B inhibits endotoxin-induced lung inflammation. Am. J. Respir. Cell Mol. Biol. 28, 373-378 (2003).
86. Matute-Bello, G., Frevert, C. W. & Martin, T. R. Animal models of acute lung injury. Am. J. Physiol. Lung Cell Mol. Physiol. 295, L379-99 (2008).
87. Williams, M. C. Conversion of lamellar body membranes into tubular myelin in alveoli of fetal rat lungs. J. Cell Biol. 72, 260-277 (1977).

### Example 2: Lamellar Body Formation by Lung Surfactant Protein B

Alveolar type II cells secrete pulmonary surfactant, a mixture of phospholipids and proteins required for breathing. Surfactant is initially stored in lamellar bodies (LB), lysosome-related organelles containing concentric dense membrane layers. LB formation depends on surfactant protein B (SP-B), a protein synthesized as a precursor (pre-proSP-B) containing three homologous domains. How LBs are formed is unclear. Here, we show that the purified middle domain of proSP-B, reconstituted into liposomes, generates LB-like structures consisting of a proteinaceous “projection core” and emanating bubbles of stacked membrane sheets. The N-terminal domain binds and transfers phospholipids, which is required for the intracellular sorting of proSP-B, the generation of lipoprotein particles in an endosomal compartment, the formation of LBs in alveolar cells, and the viability of mice after birth. Our results suggest that the N-terminal domain of proSP-B transfers phospholipids to the middle domain which, after proteolytic cleavage, generates the concentric stacked membranes of LBs.

### Introduction

Pulmonary surfactant is a mixture of proteins and phospholipids secreted by alveolar type II epithelial cells (reviewed in (Bernhard, 2016; Lopez-Rodriguez et al., 2017)). Surfactant forms a thin extracellular layer, which drastically reduces surface tension, thus facilitating breathing and preventing alveolar collapse. The air-liquid interface is thought to be lined by a monolayer of phospholipids, which is derived from a lipid bilayer that is initially stored inside the cells in lamellar bodies (LBs). LBs are lysosome-like organelles, in which membrane sheets are densely stacked on top of each other like onion layers. Upon stimulation of exocytosis, the limiting membranes of LBs fuse with the plasma membrane, releasing the internal membrane sheets to the extracellular space.

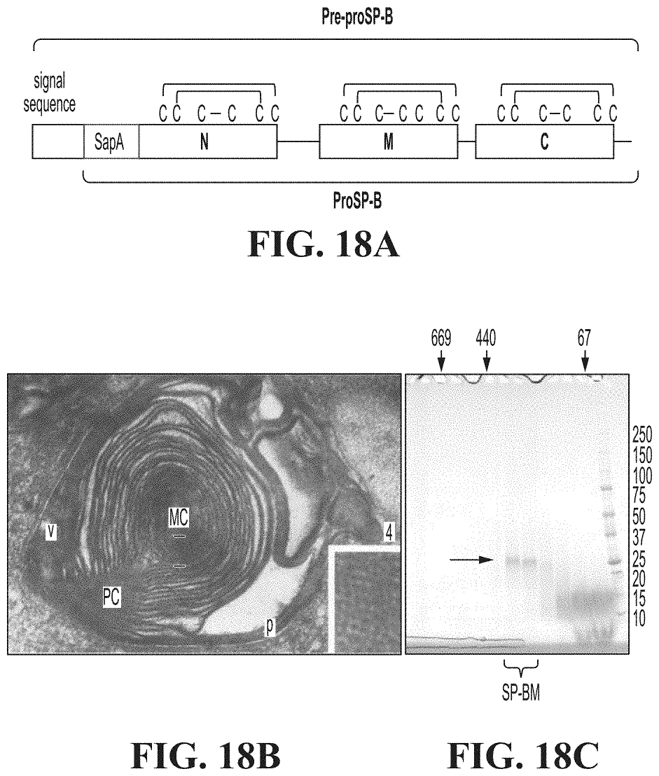

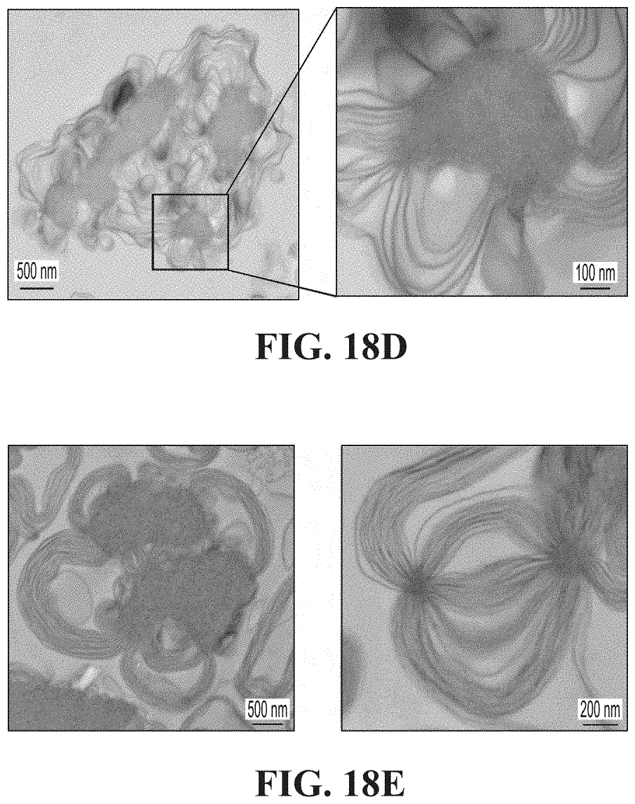

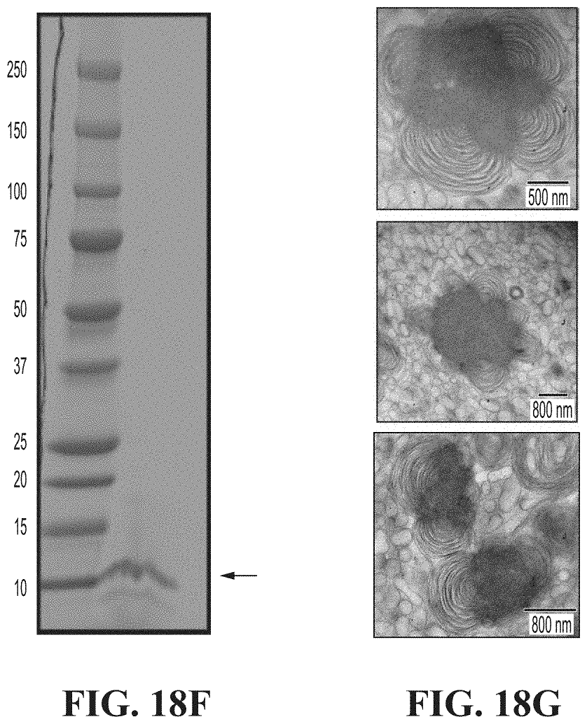

Among the four known surfactant proteins (SPs), only protein B (SP-B) is essential for breathing (for review, see (Clark et al., 1995; Nogee et al., 1993)). SP-B is a conserved protein that is found in all animal species with lungs. SP-B deficiency in mice causes neonatal lethality despite normal embryonic development (Clark et al., 1995). Adult mice expressing SP-B under a doxycycline-inducible promoter die within five days after doxycycline withdrawal (Ikegami et al., 2005; Melton et al., 2003). In humans, there are rare homozygous cases in which the gene for SP-B is mutated and the protein is not made, resulting in severe respiratory problems that generally require lung transplantation (Nogee et al., 1993; 2000). SP-B is not only involved in reducing the surface tension as a component of the extracellular surfactant, but is also essential for the formation of LBs (Clark et al., 1995; Stahlman et al., 2000). It remains unclear how exactly SP-B affects the biogenesis of LBs. SP-B is made as a precursor (pre-proSP-B), consisting of a signal sequence, a SapA domain, and three related domains with conserved disulfide bridges, called SP-BN, SP-BM, and SP-BC (Olmeda et al., 2013) (FIG. 18A). These domains are sequence-related to saposins, which are also made as a precursor that contains four, rather than three, related domains (saposins-A, -B, -C, -D). Saposins are present in lysosomes of all cells. They are lipid-binding proteins that extract lipids from luminal vesicles of multi-vesicular bodies (MVBs), so that they can be degraded by lipases (Bruhn, 2005; Kolter and Sandhoff, 2005; Vaccaro et al., 1999).

The biosynthesis of SP-B begins with its translocation into the endoplasmic reticulum (ER) lumen (Lin et al., 1996a; 1996b). The signal sequence is removed after translocation and the resulting proSP-B protein is transported in vesicles to the Golgi apparatus, where it is diverted from the secretory pathway to an endosomal compartment. MVBs are thought to serve as direct precursors of LBs. On its way to LBs, proSP-B is sequentially processed by several lysosomal proteases into the individual SP-B domains (Gerson et al., 2008; Korimilli et al., 2000; Ueno et al., 2004). The middle domain (SP-BM) is considered to be the mature protein that is important for LB formation and surfactant function. In some species, such as humans, SP-BM is concentrated in an electron-dense region of the LBs, called the “projection core”, from which the membrane stacks seem to emanate (Stratton, 1978). In other species, projection cores are not evident and SP-BM is found throughout the membrane stacks (Brasch et al., 2004). The N-terminal domain (SP-BN) has been reported to have anti-microbial activity (Yang et al., 2010), but its exact function remains unclear. The C-terminal domain (SP-BC) does not seem to be essential for breathing, as a transgene lacking this domain rescues SP-B knock-out mice (Akinbi et al., 1997).

Here, we have analyzed the functions of the SP-B domains in LB formation. We use crystallography, biochemical assays, and experiments with cultured cells and mice to derive a model for LB formation. Our results also have implications for the generation of a surfactant mixture for use in the treatment of respiratory distress in patients.

### Results

### Reconstituted SP-BM Generates LB-Like Structures

The SP-B precursor is proteolytically processed into three saposin-like proteins. We first addressed the function of the middle (SP-BM) domain. SP-BM has been detected in human LBs in a dense protein-rich area, called the projection core (PC) (Stratton, 1978). Thin-section electron microscopy (EM) performed more than 40 years ago showed that the concentric membrane sheets, characteristic of LBs, seem to emanate from or merge at the PC (FIG. 18B) (Stratton, 1978). Based on these results, we tested whether LB-like structures can be formed by SP-BM alone.

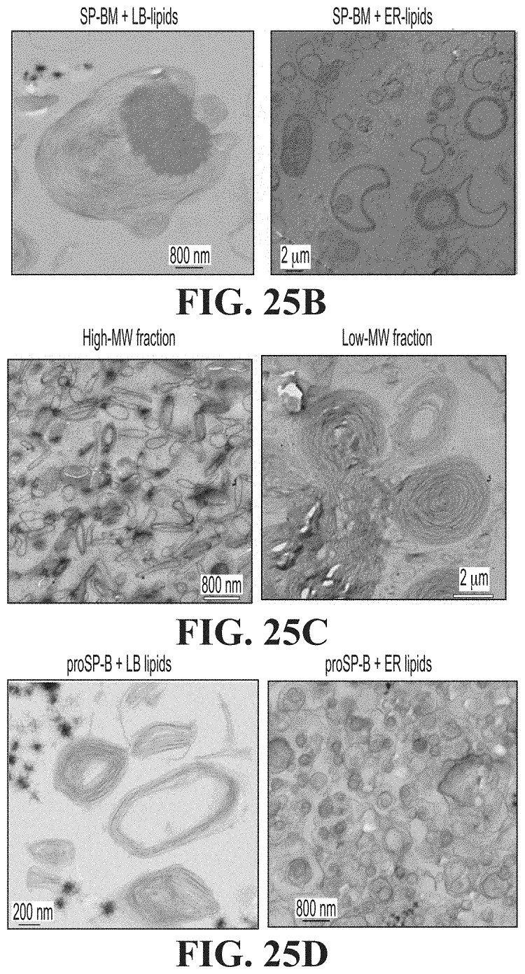

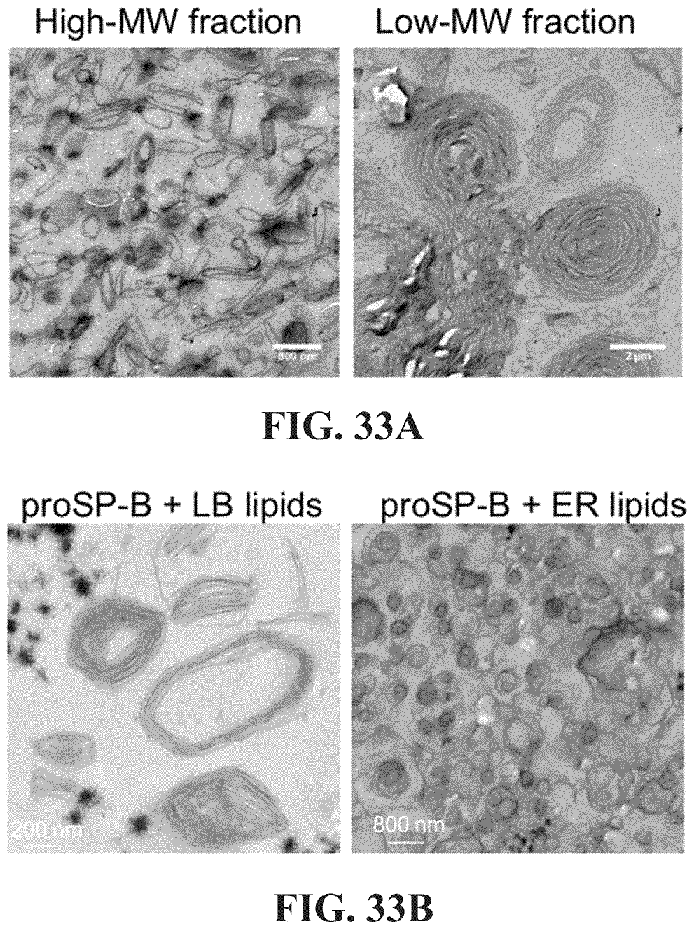

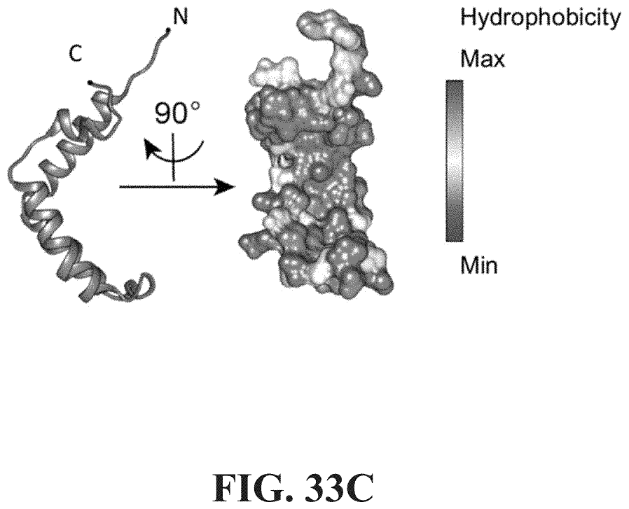

We initially purified SP-BM from bovine lungs, taking advantage of the fact that it is one of a few proteins that are soluble in chloroform/methanol (Mathialagan and Possmayer, 1990). The extract was subjected to chromatography on a Sepharose LH-20 column to remove phospholipids. Protein fractions were dried, dissolved in the detergent octylglucoside, and further purified by size-exclusion chromatography (SEC). Non-reducing SDS-PAGE of the purified protein showed a single band corresponding in size to a dimer (FIG. 18C), as expected from the fact that SP-BM monomers are disulfide-linked through Cys48 (Beck et al., 2000). Purified SP-BM dimer was then mixed with a dried phospholipid (PL) mixture consisting of dipalmitoylphosphatidylcholine (DPPC) and phosphatidylglycerol (PG) (2:1), mimicking the composition of LBs (King, 1982). The detergent was removed by dialysis, and the proteoliposomes were sedimented and analyzed by osmium staining and thin-section EM. The observed structures strikingly resemble the images obtained from lung sections (FIG. 18B): they show electron-dense areas similar to projection cores from which bubbles of stacked membrane sheets seem to emerge (FIG. 18D). Because LBs contain a high Ca2+ ion concentration (Chintagari et al., 2010), we also added Ca2+ after dialysis. In this case, the stacking of the membrane sheets tended to be even more impressive (FIG. 18E). With increasing protein to lipid ratios, more electron-dense areas with fewer associated membrane bubbles were observed (FIG. 25A, FIG. 33A-33C), consistent with the electron-rich regions containing SP-BM. Electron-dense areas with emanating membrane bubbles were scarce or absent when SP-BM was reconstituted with a lipid mixture mimicking the ER composition (FIG. 25B). When protein was omitted, the membrane pellet was much smaller, probably because lipid was lost during dialysis.

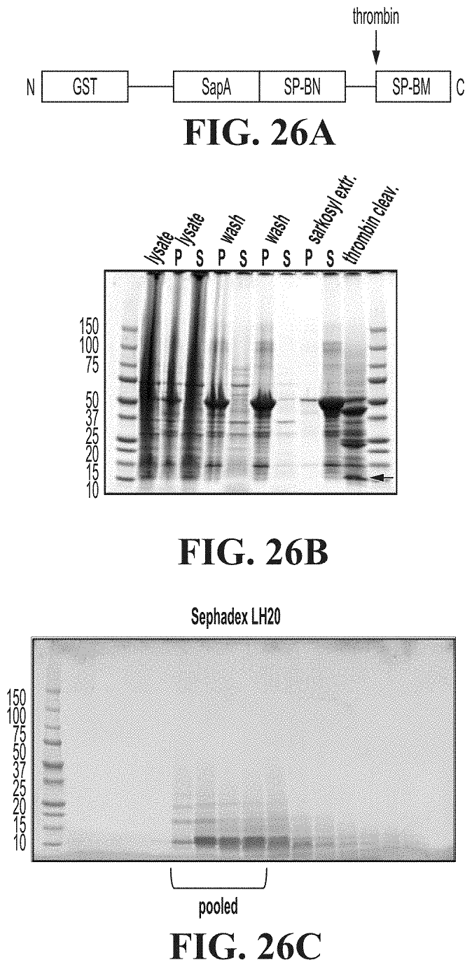

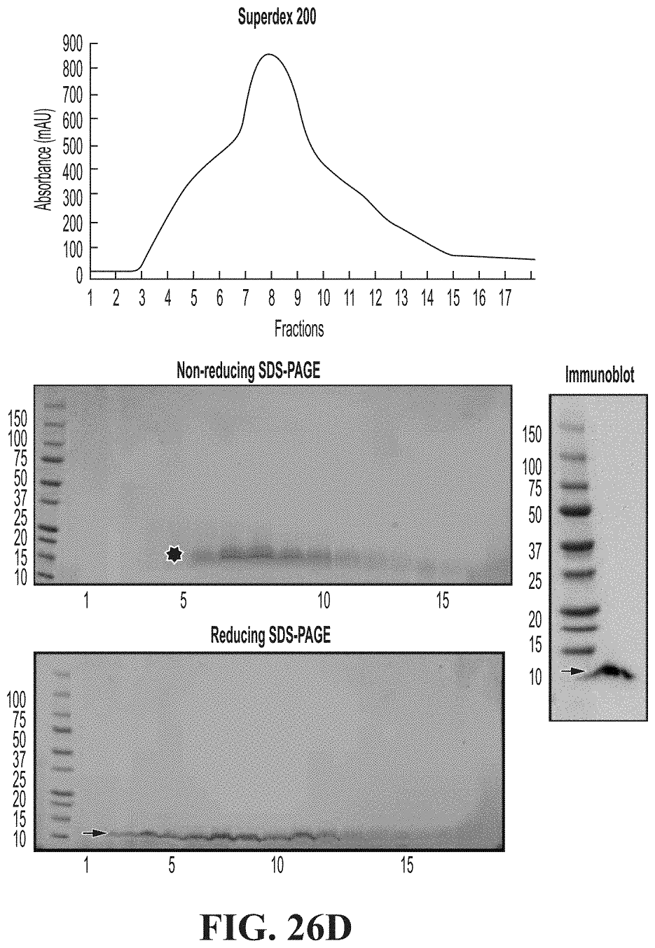

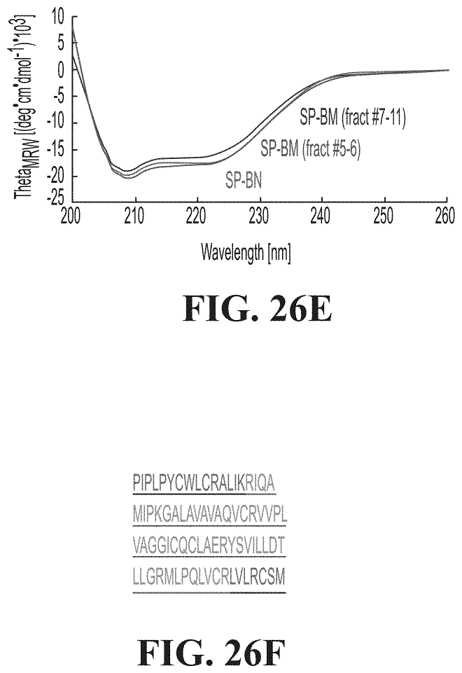

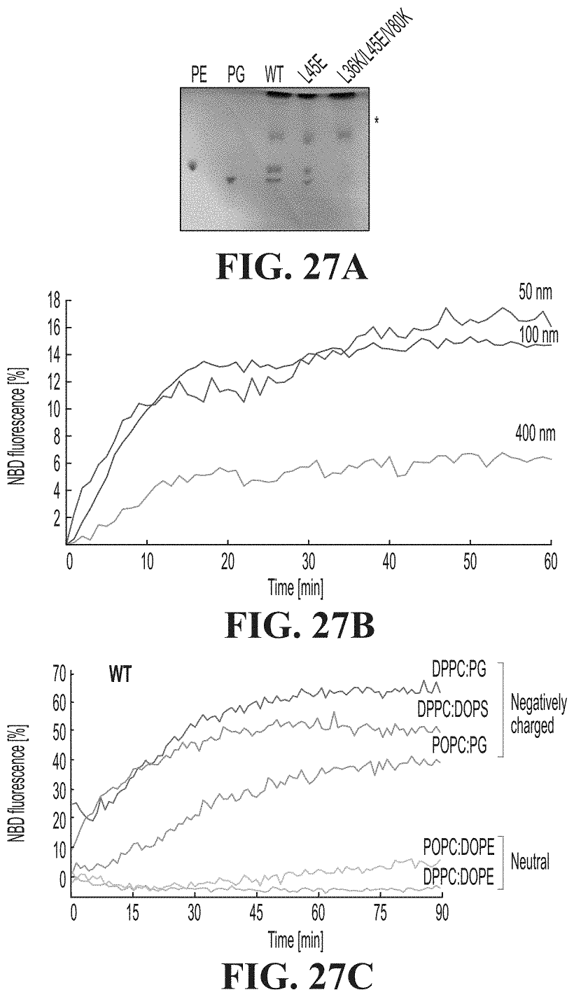

Expression and purification of recombinant SP-BM has proven difficult, but eventually we designed a protocol based on the E. coli expression of a fusion of glutathione S-transferase (GST) and human proSP-B lacking the SP-BC domain, with a thrombin-cleavage site between the SP-BN and SP-BM domains (FIG. 26A). The fusion protein formed inclusion bodies that were solubilized in the detergent sarkosyl. After thrombin cleavage, SP-BM was extracted into chloroform/methanol and the protein was separated from PLs by chromatography on a hydrophobic resin. The organic solvent was removed and the protein was solubilized in octylglucoside and further purified by SEC (FIG. 26B-D; FIG. 18F). CD spectroscopy showed that the protein is folded (FIG. 26E), and immunoblotting with SP-BM antibodies and mass spectrometry confirmed the identity of the purified protein (FIG. 26D; 26F). After reconstitution into liposomes with LB-like lipids, the dimer fraction obtained by SEC generated structures similar to those obtained with SP-BM purified from bovine lung, i.e. projection cores with emanating membrane bubbles (FIG. 18G). In contrast, the higher weight fraction gave rise to vesicles, and the lower molecular weight fraction to multi-lamellar structures without projection cores (FIG. 25C). Taken together, these results indicate that LB-like structures can be generated with reconstituted purified SP-BM.

### SP-BN is a Lipid-Binding and -Transfer Protein

Next, we analyzed the N-terminal domain of SP-B (SP-BN). Given its sequence similarity to saposins, we tested whether it has an uncharacterized lipid binding activity. SP-BN was N-terminally tagged with His6 and thioredoxin, and expressed in E. coli cells lacking thioredoxin reductase, a strain that allows disulfide-bridge formation in the cytosol (Derman et al., 1993). The fusion protein was purified on a Ni-NTA resin, and the thioredoxin moiety was cleaved off The protein was further purified by SEC and subjected to crystallization. Crystals were obtained at pH 5.4, mimicking the acidic environment in LBs (Chander et al., 1986), and diffracted to 2.2 Å resolution. The structure was solved by sulfur single-wavelength anomalous dispersion (SAD) (Table 1). The asymmetric unit contained four copies of SP-BN dimers, which all had essentially the same structure.

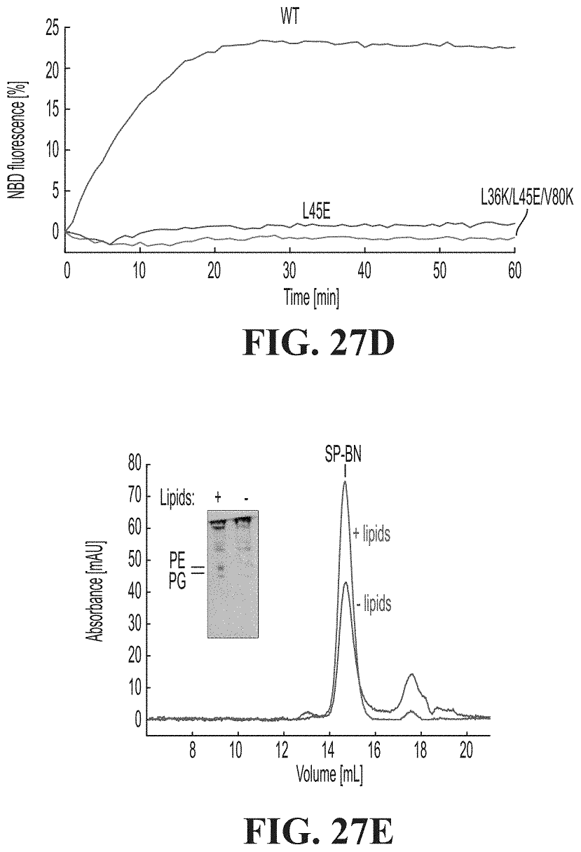

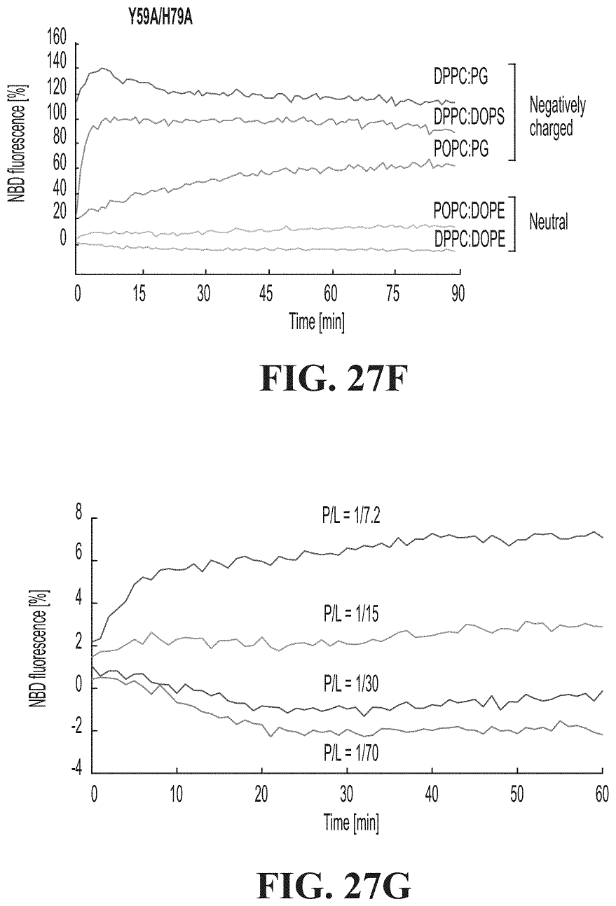

Like all saposin-like proteins, the SP-BN monomer consists of four helices with conserved intramolecular disulfide bridges and a loop between helices 2 and 3 (FIG. 19A). In different structures of saposin-like proteins, the four helices form pocket-knife structures with open or closed conformations (Olmeda et al., 2013). In our SP-BN structure, two monomers in an open conformation form a dimer with a hydrophobic hollow in between (FIG. 19A, FIG. 32A-32B). The monomers are associated through their helices 1 at the “floor”. The two loops between helices 2 and 3 contain additional short helices and form a “roof”. Inside the hydrophobic hollow are three PL molecules that originate from the E. coli cells in which the protein was expressed. Thin-layer chromatography (TLC) (FIG. 27A) and mass spectrometry (Table 2) confirmed that the PL composition is the same as in E. coli, consisting mostly of phosphatidylethanolamine (PE) and PG. We modeled the PL molecules in the density map with the most abundant species (Table 2), i.e. PE molecules with acyl chains of 16 and 18 carbon atoms, each containing one double-bond. The density for the phospholipid head groups was ill-defined, indicating that they are heterogeneous or weakly bound.

Two of the PL molecules are bound laterally inside the cavity and have their head groups on opposite ends of the hollow (FIG. 19B). The acyl chains of both PL molecules curl around and bind into a pocket of SP-BN. The central PL molecule has hydrocarbon chains that extend across the entire length of the hollow, with one chain nearly straight and the other forming a kink that may represent a double bond (FIG. 19A). The occupancy in the crystal structure indicates that the head group of this PL molecule can be on either end of the hollow. Importantly, the hydrocarbon chains of this PL molecule only make contact with the hydrocarbon chains of the lateral PL molecules, and not with the protein. The structure immediately suggests that SP-BN is a PL transfer protein, which allows the central PL molecule to move with minimal energetic costs between SP-BN and a phospholipid bilayer. Positively charged amino acid residues at the roof may be involved in the interaction with the negatively charged lipid bilayer (FIG. 19C) and several hydrophobic residues generate the hydrophobic interior of the hollow (FIG. 19D). The head groups of the bound PLs might be stabilized by interaction with residues Y59 and H79 at the two ends of the hollow (FIG. 19E). These predictions are tested in the next section.

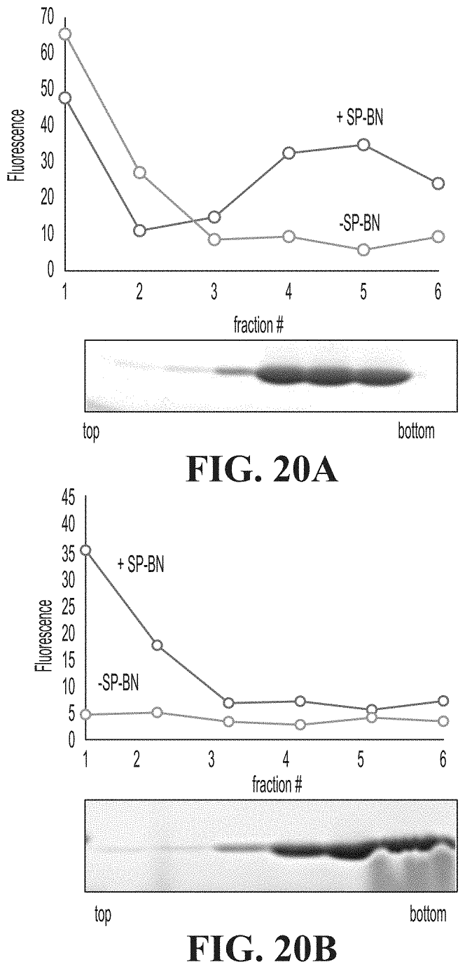

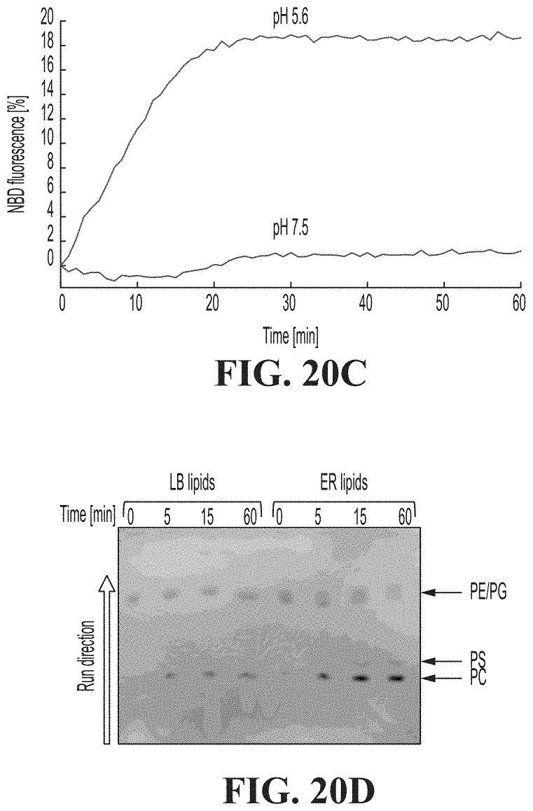

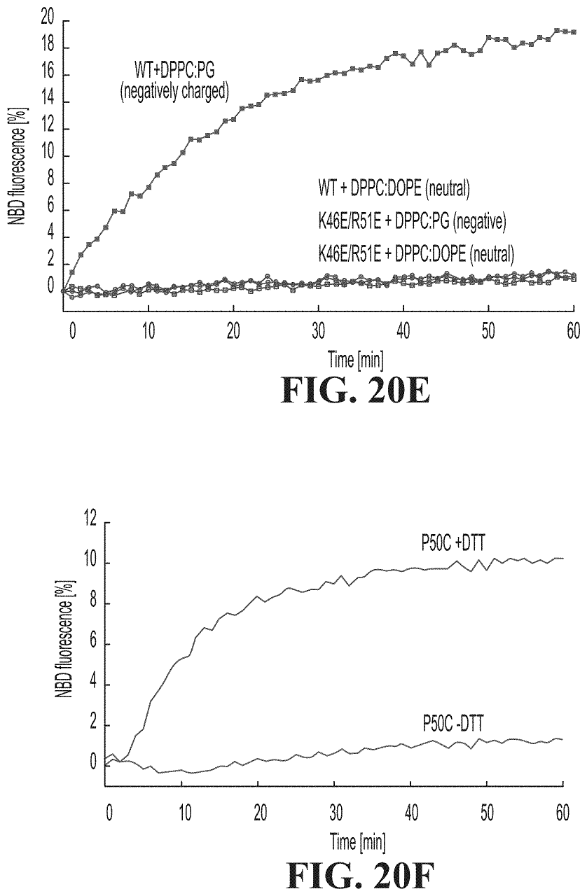

To test whether SP-BN mediates lipid transport between lipid bilayers, we mixed purified SP-BN with liposomes containing fluorescently labeled PLs and floated the vesicles in a Nycodenz gradient (FIG. 20A). In the presence of SP-BN, some of the fluorescent lipid was retained in the bottom fractions together with the protein, indicating lipid transfer from the vesicles to the protein. The bottom fractions were then mixed with excess of unlabeled liposomes and subjected to another round of flotation. Now, all the fluorescent lipid floated to the top of the gradient (FIG. 20B), indicating lipid transfer from SP-BN to the added liposomes. SDS-PAGE showed that SP-BN does not float with the vesicles (FIG. 20A-20B; bottom panels), indicating that it has only weak affinity for lipid bilayers.

To follow lipid transfer kinetically, we used fluorescently labeled PLs in a dequenching assay. Emission of 7-nitrobenzoxadiazole (NBD) fluorescence of NBD-PE is initially quenched by rhodamine attached to PE, but when the fluorescent lipids are transferred to unlabeled liposomes or SP-BN protein, an increase in NBD emission is observed (Bian et al., 2011). Lipid transfer was observed at low, but not neutral pH (FIG. 20C), consistent with SP-BN functioning in an endosomal compartment. Lipid transfer was most efficient with liposomes having a diameter smaller than 100 nm, indicating that high membrane curvature facilitates transfer (FIG. 27B).

To demonstrate that SP-BN transfers biologically relevant lipids and not merely fluorescent ones, we developed a filtration assay. Using centrifugation through 100-kDa molecular weight cut-off filters, we found that SP-BN moves into the filtrate, while liposomes stay behind. The lipids were then extracted from the filtrate with chloroform/methanol and subjected to TLC. With this assay, we found that SP-BN picks up PLs from liposomes with either LB- or ER-like lipid composition (FIG. 20D). Little or no lipid transfer was observed at 0° C. Taken together, these experiments indicate that SP-BN is a non-specific phospholipid-binding and -transfer protein.

### Mechanism of Phospholipid Transfer by SP-BN

We used the fluorescence dequenching assay to analyze the mechanism of lipid transfer by SP-BN. Lipid transfer was observed with liposomes containing negatively charged, but not neutral PLs (FIG. 20E; FIG. 27C). This is due to an interaction of the negatively charged PL head groups with the positively charged amino acids that protrude from the “roof” of the SP-BN dimer (K46, R51) (FIG. 19C). Mutation of these residues to negatively charged amino acids (K46E/R51E) abolished lipid transfer in the fluorescence-dequenching assay (FIG. 20E), but the protein still purified with bound lipids. A crystal structure of this mutant at 1.9 Δ resolution was essentially identical to that of wild-type SP-BN (FIG. 19F; Table 1), although the mutant crystallized in a different space group with only two dimers in the asymmetric unit. The dimer still contained all three PL molecules in approximately the same conformations (FIG. 19G). Thus, the K46E/R51E mutant transfers PLs more slowly than the wild-type protein, but ultimately binds them in the same way.

Our structure suggests that PLs enter and exit the hollow of the SP-BN dimer through the roof We tested this model by mutating P50 to a cysteine, so that the roof would be locked when a disulfide bridge is formed between the loops of the two monomers (FIG. 20C). The P50C mutant was indeed inactive in lipid transfer, but became active when the disulfide bridge was reduced with dithiothreitol (DTT) (FIG. 20F).

The crystal structure indicates that three residues (L36, L45, and V80) make a major contribution to the hydrophobic interior of the PL-binding hollow (FIG. 20D). When these residues were mutated (L36K/L45E/V80K mutant), PL was no longer bound (FIG. 27A) and no lipid transfer was seen in the fluorescence-dequenching assay (FIG. 27D). The single L45E mutation abolished lipid transfer, but did not abrogate lipid binding (FIGS. 27A, 27D). The triple-mutant could be purified similarly to the wild-type protein, suggesting that lipid binding is not required for the folding of SP-BN. This is supported by the observation that, upon detergent extraction of bound lipids, the wild-type protein still eluted at the same position in SEC (FIG. 27E).

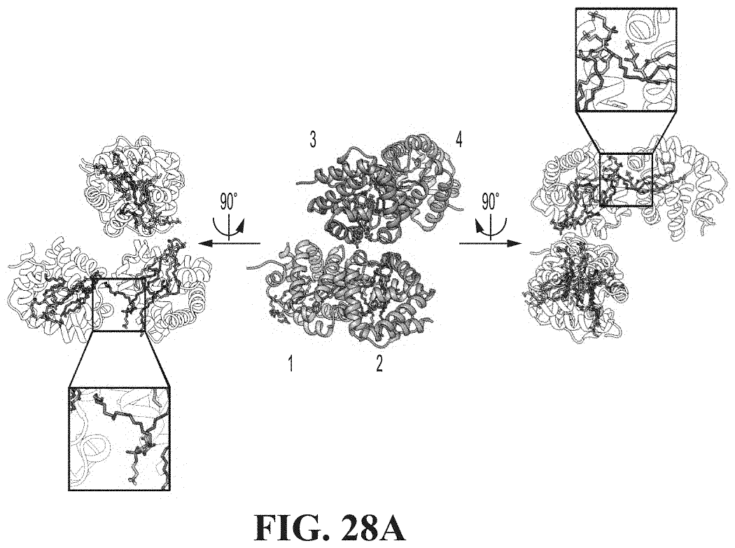

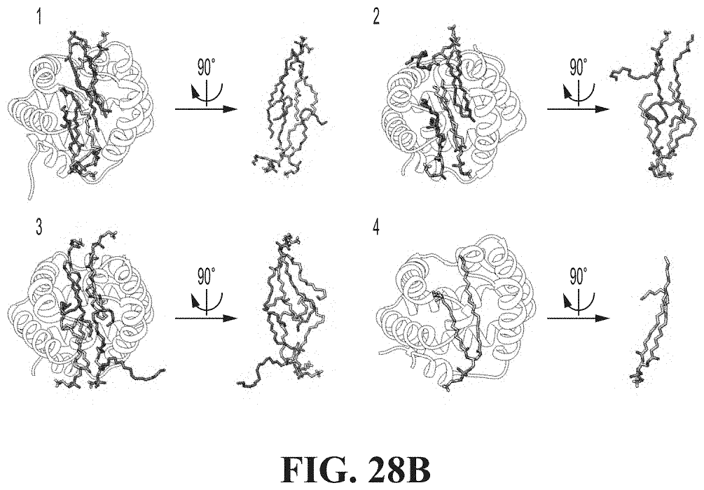

Our structure also predicts that the head groups of the bound PLs are stabilized by interaction with Y59 and H79, located at the two ends of the hollow (FIG. 19E). Mutant Y59A/H79Δ still purified with bound lipid and was active in lipid transfer (FIG. 27F). We determined a crystal structure of this mutant and found that the asymmetric unit contained four copies of the SP-BN dimer (FIG. 28A; Table 1), like the wild-type protein. The structures of all SP-BN molecules remained the same, consistent with the mutated residues pointing outwards (FIG. 19E). However, the four SP-BN dimers no longer had identically bound PL molecules (FIG. 28B). Whereas in the wild-type protein all four dimers had six acyl chains in the hollow (two from each PL molecule), in the mutant the number varied from three to seven (FIG. 28B). Some of the acyl chains were no longer inside the hollow (FIG. 28A). These results indicate that Y59 and H79 are required for the correct positioning of the three PL molecules. They also indicate that the hollow can accommodate a variable number of acyl chains, consistent with the idea that, during lipid transfer, it must transiently contain fewer than six acyl chains.

Although the isolated SP-BN domain binds and transfers PL, very high protein to lipid ratios (more than 1:10 molar ratio) were required (FIG. 27G). This suggests that its activity is normally stimulated by other factors (see below).

### Crystal Structures of SP-BC

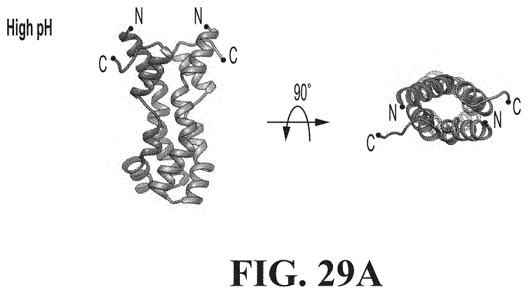

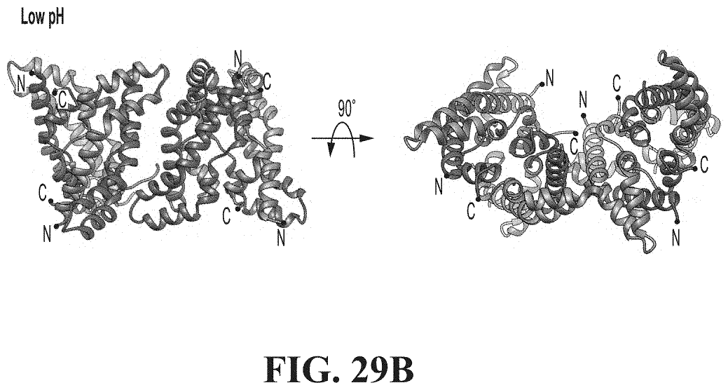

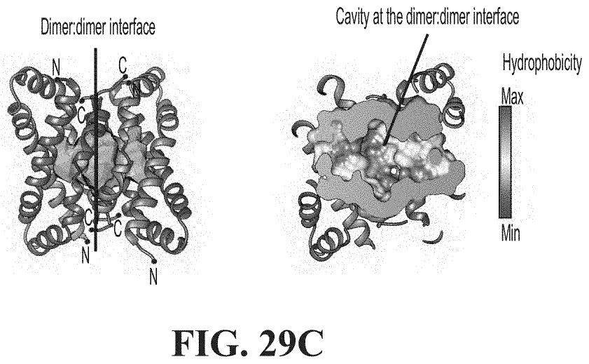

We purified SP-BC in the same way as SP-BN, but it did not contain bound PL and was inactive in our PL transfer assays. Crystal structures of SP-BC were obtained at high and low pH, diffracting to 1.75 Å and 1.90 Å, respectively (Table 1). In the high-pH structure, SP-BC forms a dimer, in which the two monomers interact through straight helix pairs and are oriented in a parallel manner (FIG. 29A). In the low-pH structure, the monomers are anti-parallel and form a dimer, again with the monomers in a straight conformation; two of the dimers form a tetramer with a moderately hydrophobic pocket (FIG. 29B; 29C). Although there is some weak density in the pocket, it is unclear whether it originates from a low-occupancy lipid or another molecule.

Lipid Binding to the SP-BN Domain Determines Intracellular Trafficking of proSP-B.

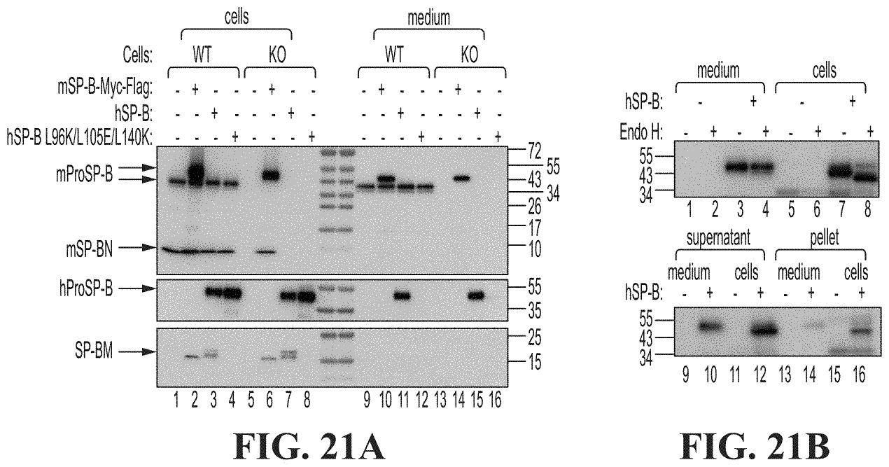

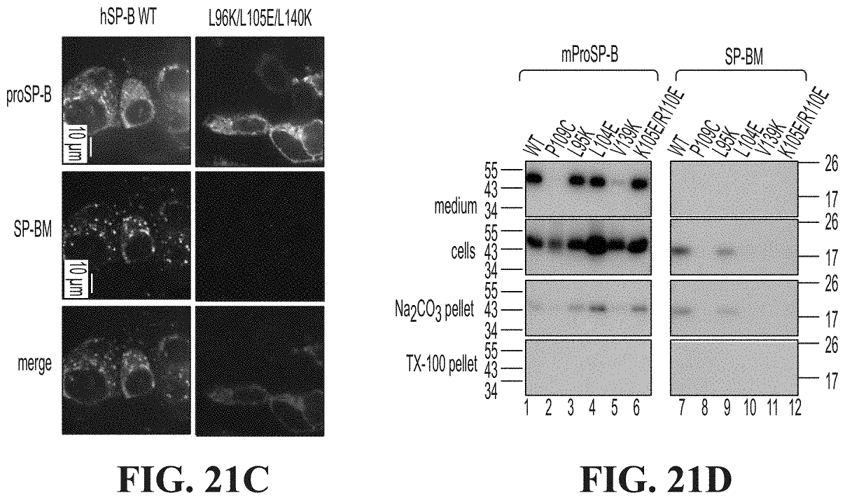

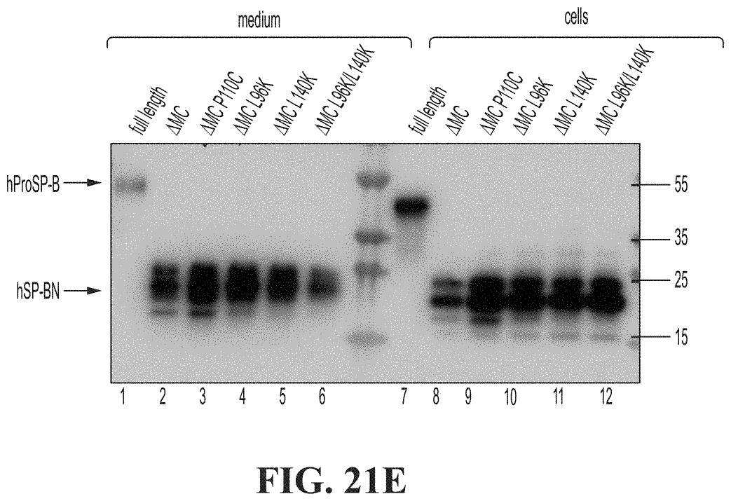

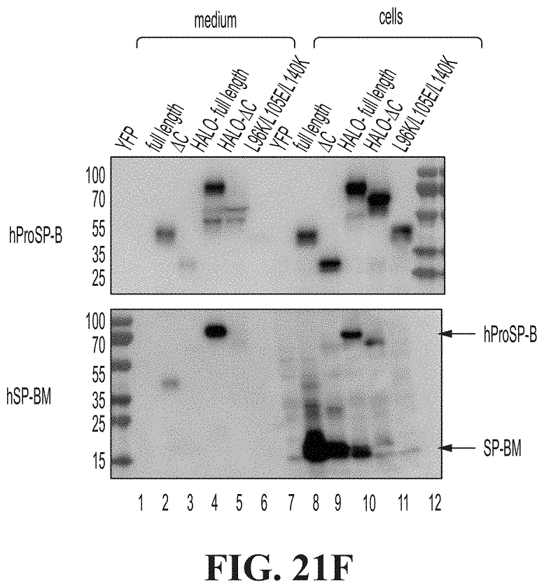

To test the physiological role of SP-BN's lipid-binding and -transfer activity, we used an established mouse alveolar epithelial cell line (MILE-15) (Wikenheiser et al., 1993). These cells have LBs, albeit with fewer membrane stacks than in mouse lungs. Immunoblotting with antibodies raised against mouse SP-BN detected endogenous proSP-B, the precursor of SP-B lacking the signal sequence, both in the cellular membrane fraction and the medium (FIG. 21A, lanes 1 and 9). Mature SP-BN was only detected inside the cells and mature, endogenous SP-BM was undetectable in most experiments (lanes 1 and 9). Transient expression of a C-terminally tagged version of mouse SP-B (mSP-B-Myc-Flag) again showed proSP-B (which runs slower in SDS gels because of the tag) both in cells and the medium (FIG. 21A, lanes 2 and 10). Mature SP-BM was now detectable and found only inside the cells (lanes 2 and 10). Similar results were obtained when untagged human SP-B (hSP-B) was transiently expressed (lanes 3 and 11); in this case, a different antibody had to be used to detect proSP-B.

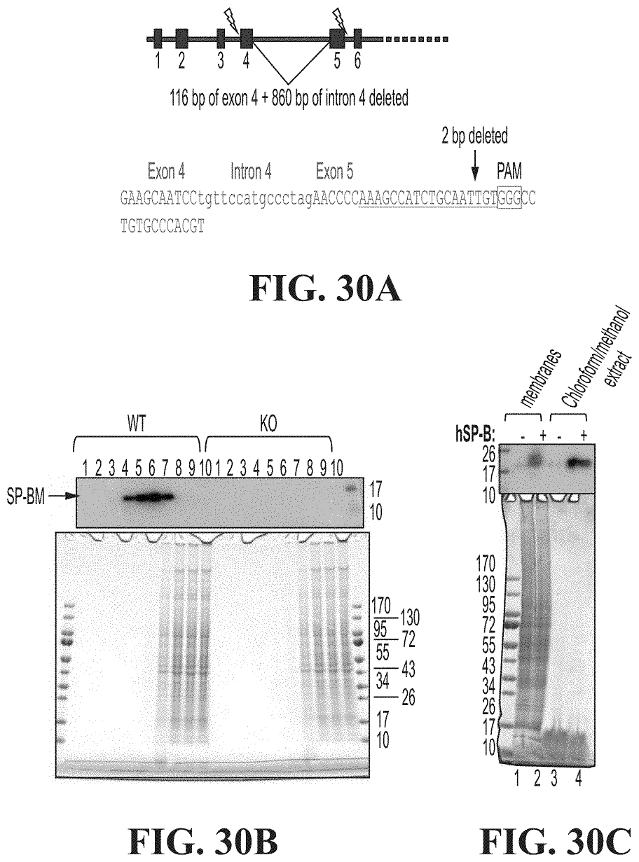

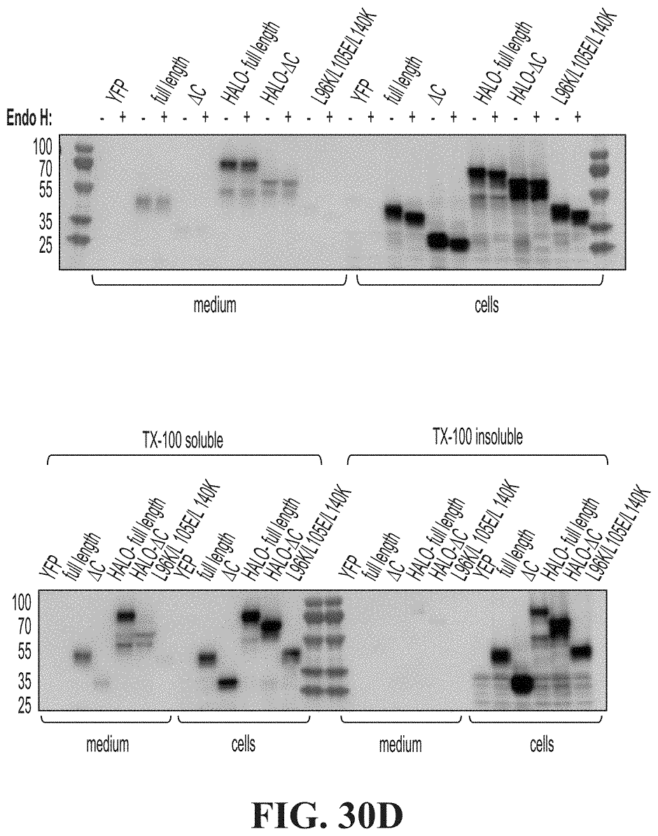

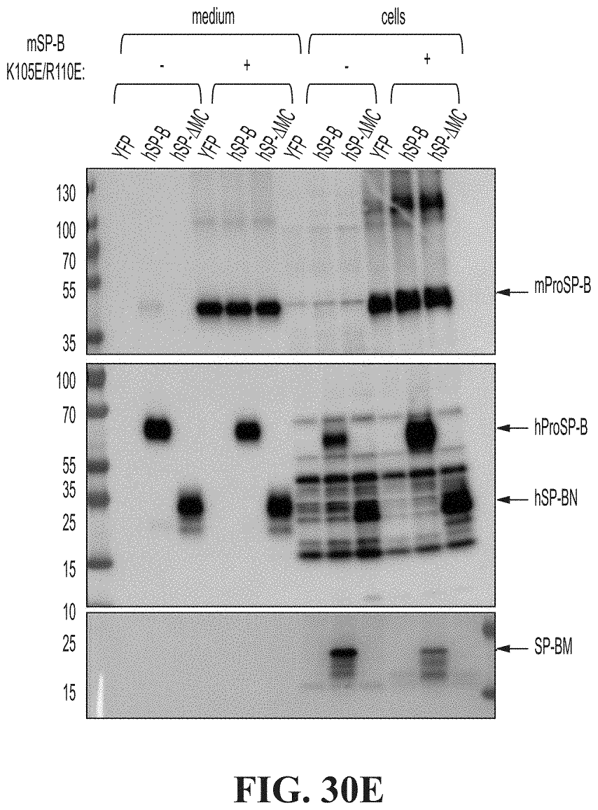

To avoid confusion with endogenous SP-B, we generated a SP-B knock-out MLE-15 cell line by CRISPR. One of the knock-out clones not only had most of exon 4 and intron 4 deleted, but also had two nucleotides deleted in exon 5 (FIG. 30A). Accordingly, no SP-B protein was detected in the cells or medium (FIG. 21A, lanes 4 and 13). Transient expression of mSP-B-Myc-Flag or hSP-B in the knock-out (KO) cells again showed proSP-B in the cells and medium, and mature SP-BM only inside the cells (lanes 6, 7 and 14, 15).

Using transiently expressed hSP-B, we found that intracellular proSP-B contained glycans sensitive to endoglycosidase H (endo H) treatment (FIG. 21B, lanes 7versus 8), indicating its ER localization. The secreted proSP-B population was endo H resistant (FIG. 21B, lanes 3 and 4). ER localization of intracellular proSP-B is supported by immunostaining with an antibody that exclusively recognizes proSP-B (FIG. 21C). The ER-localized population is not grossly misfolded, as it is soluble in Triton X-100 (FIG. 21B, lane 12 versus 16). Immunostaining with an antibody to SP-BM showed that mature hSP-BM is localized to puncta (FIG. 21C), consistent with its localization to LBs. This is supported by sucrose gradient centrifugation experiments, in which mature SP-BM was found in a light membrane fraction (FIG. 30B). Furthermore, the protein was extractable in chloroform/methanol (FIG. 30C), similar to SP-BM from lungs. Taken together, these experiments show that in MILE-15 cells, a fraction of proSP-B is exported from the ER. This population is again divided into one that is secreted and another that is proteolytically processed and transported to LBs.

We next introduced into the hSP-B precursor the three mutations that abrogated lipid binding to SP-BN (L36K/L45E/V80K; corresponding to L96K/L105E/L140K in proSP-B). The triple-mutant was found inside the cells, but not the medium (FIG. 21A, lanes 4, 8 and 12, 16); no mature SP-BM was detectable (FIG. 21B, lanes 4, 8). EndoH sensitivity and immunostaining confirmed that mutant proSP-B stayed in the ER and was not processed to SP-BM (FIG. 21C; FIG. 30D). As for the wild-type protein, a significant percentage of the ER-localized mutant proSP-B population was soluble in Triton X-100 (FIG. 30D), indicating that it did not aggregate. ER-retention was also observed with proSP-B carrying only one of the three mutations (V80K in SP-BN, corresponding to V139K in proSP-B) (FIG. 21D, lanes 5 and 11). In contrast, the single-mutant equivalent to L36K (L95K in proSP-B) behaved like wild-type, as some population of proSP-B was secreted and another processed into mature SP-BM (FIG. 21D, lanes 3 and 9). Results similar to those with the L36K/L45E/V80K and V80K mutants were obtained with the P50C mutant (P109C in proSP-B; FIG. 21D, lanes 2 and 8), a mutation that greatly reduced lipid transfer activity of the isolated SP-BN protein (FIG. 20F). All mutant proSP-B proteins were expressed at similar levels to wild-type proSP-B and their intracellular population was extractable with TX-100 or alkali (FIG. 21D; compare pellets before and after extraction), indicating that the mutations did not cause aggregation or membrane integration. Taken together, these results show that lipid binding to the SP-BN domain is required for export of proSP-B from the ER.

Interestingly, the lipid binding mutants were able to leave the ER if both the SP-BM and SP-BC domains were deleted (constructs labeled SP-ΔMC) (FIG. 21E). For example, the mutations P109C or L139K in proSP-B prevent ER export in the full-length protein (FIG. 21D, lanes 2 versus 8 and 5 versus 11), whereas these mutations allow a fraction of the truncated protein to be secreted (FIG. 21E, lanes 3 versus 9 and 5 versus 11). Thus, lipid binding to SP-BN is required for ER exit of proSP-B, but not of SP-BN itself.

Next, we introduced mutations into mouse proSP-B that reduce lipid-transfer, but not—binding, of isolated SP-BN (FIG. 20E, FIG. 19F). A proSP-B mutant equivalent to K46E/R51E in SP-BN (K105E/R110E in proSP-B) was localized to both the ER and medium, but was only inefficiently processed into mature SP-BM (FIG. 21D, lanes 6 and 12; reduced to ˜10% of wild-type levels). A similar phenotype was observed with the L45E mutant (L104E in proSP-B) (FIG. 21D; lanes 4 and 10; FIG. 27D). In both cases, the mutations are at the roof of the SP-BN dimer. These results show that proSP-B needs efficient lipid transfer activity for its targeting to the LB pathway. Co-expressing the mouse K105E/R110E mutant with wild-type human N-terminal domain (hSP-ΔMC) did not rescue the processing of mutant precursor (FIG. 30E), indicating that lipid transfer needs to occur with SP-BN and SP-BM domains present in the same polypeptide chain.

Finally, we found that the SP-BC domain stimulates export of proSP-B from the ER. When this domain was deleted from wild-type proSP-B (construct labeled AC), most of the protein stayed inside the cells; little was secreted or proteolytically processed into the mature SP-BM domain (FIG. 21F; lane 3 versus 9). Similar results were obtained when the constructs carried HALO tags at their N-termini (FIG. 21F; lane 4 versus 10). All intracellular proteins were soluble in Triton X-100 and endoH sensitive (FIG. 30D), indicating that they were located in the ER, but not aggregated. Together, our results indicate that both lipid-binding to the SP-BN domain and the presence of the SP-BC domain are required for efficient export of proSP-B from the ER.

### ProSP-B has Increased Lipid Transfer Activity and Forms Lipoprotein Particles

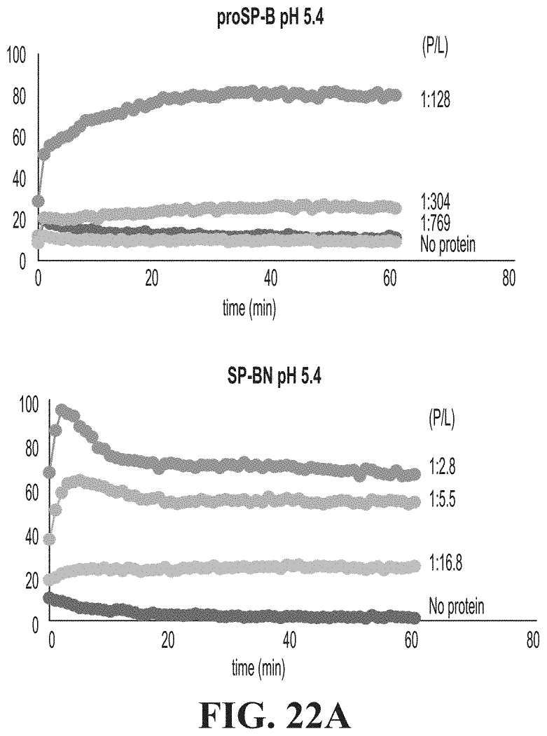

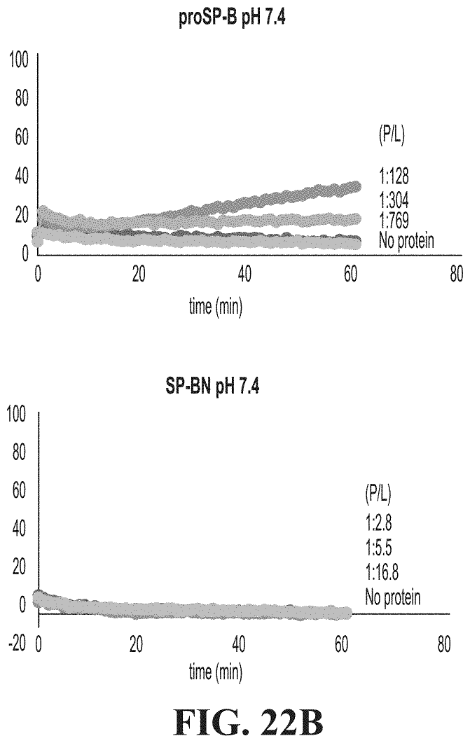

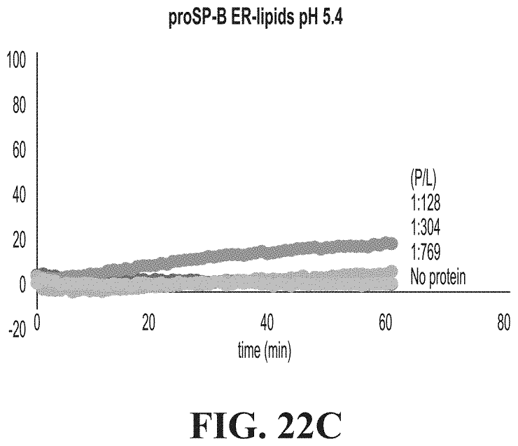

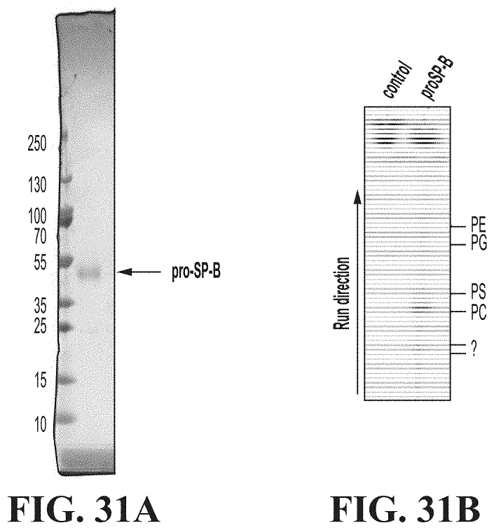

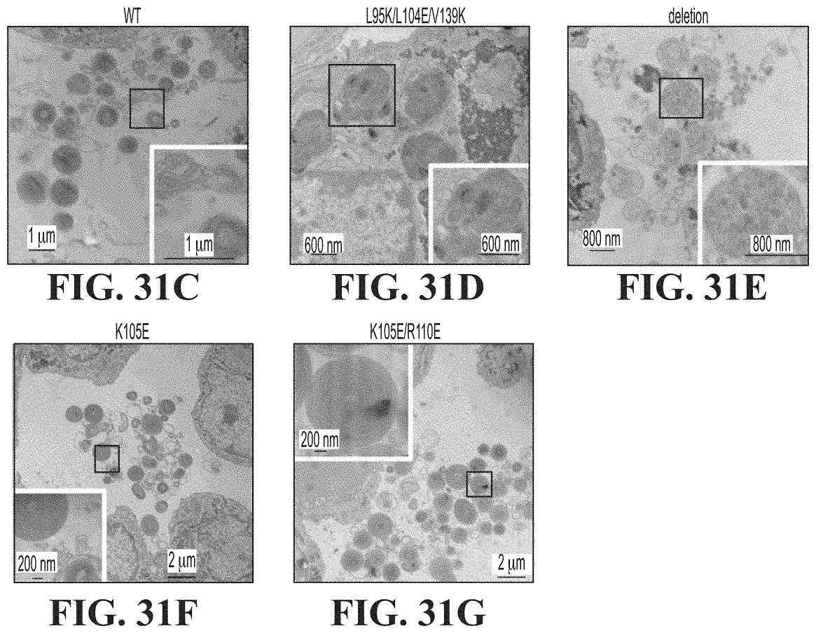

To test whether proSP-B has lipid transfer activity, we purified the protein with an N-terminal HALO-tag from the medium of HEK293 cells that stably express the fusion protein. After removal of the tag, the purified protein migrated as a single band in SDS-PAGE (FIG. 31A) and contained PC and some unidentified more hydrophilic lipid (FIG. 31B). Purified proSP-B showed lipid transfer activity in the fluorescence dequenching assay at 20 to 30-fold lower concentrations than purified SP-BN (FIG. 22A). Transfer activity was reduced at neutral pH (FIG. 5B) and with liposomes containing ER-like, instead of LB-like, PLs (FIG. 22C). Both observations suggest that proSP-B transfers lipids in a low-pH, LB-like compartment (Chander et al., 1986).

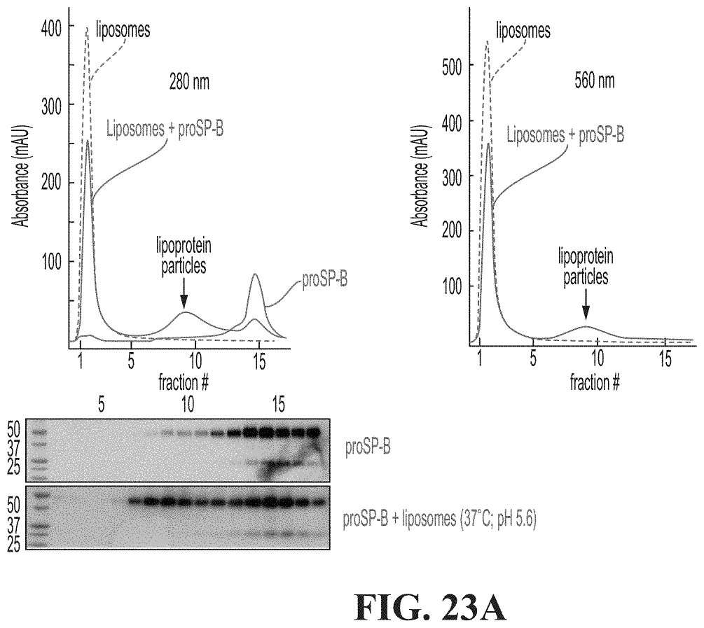

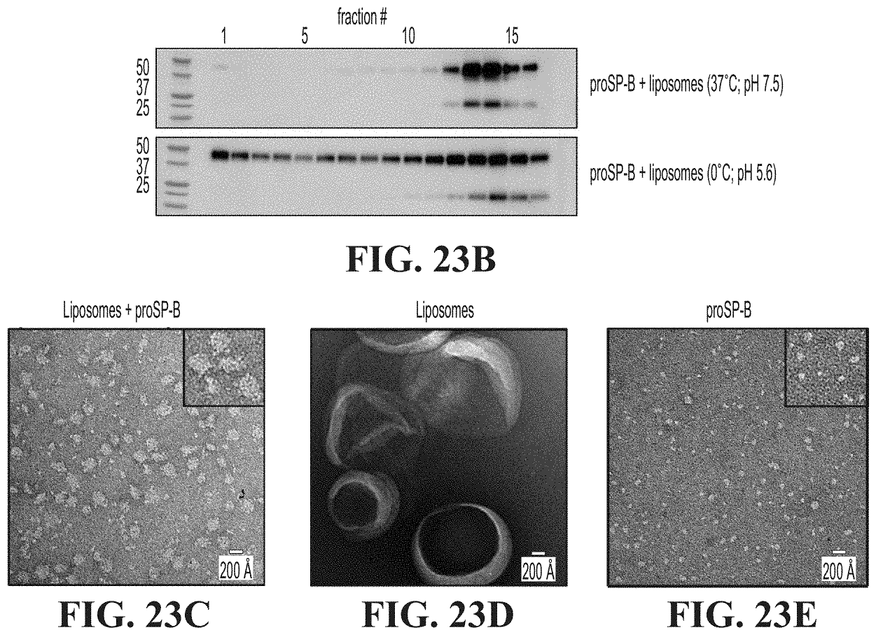

ProSP-B converts liposomes into lipoprotein particles: When purified proSP-B was incubated at low pH and 37° C. with preformed liposomes containing a LB-like PL composition and fluorescent lipid, a fraction of proSP-B shifted its elution volume in SEC (FIG. 23A). In the absence of liposomes, proSP-B eluted mostly as a dimer, although some higher oligomers were also observed. Upon incubation with liposomes, a sizable fraction of proSP-B eluted at a size between that of the protein alone and of the original liposomes (FIG. 23A). This population also contains PLs, as shown by the absorbance of the fluorescent lipid at 560 nm (FIG. 23A). To estimate the lipid to protein ratio in the lipoprotein particles, we determined the amount of fluorescent lipid in these fractions by either measuring the absorbance at 560 nm or the fluorescence in a plate reader. The amount of proSP-B in these fractions was determined on the basis of a comparison with a titration of purified proSP-B in immunoblots. Together, these data indicate that each proSP-B monomer binds 15-25 PL molecules. Lipoprotein particle formation was not seen at neutral pH (FIG. 23B), consistent with proSP-B acting in an endosomal compartment. Furthermore, when the incubation was performed at 0° C., some proSP-B eluted together with the liposomes, indicative of binding, but a distinct lipoprotein peak was not observed (FIG. 23B).

Negative-stain EM showed that the lipoprotein fraction consisted of particles with a variable diameter (˜20-35 nm) (FIG. 23C). Their size and rugged surface confirm that they are not vesicles with a normal phospholipid bilayer. Both the negative-stain EM and SEC data indicate that the particles have a molecular mass above 0.6 million, which means that they contain about 12 molecules proSP-B and 180 molecules PLs. These lipoprotein particles were not seen with liposomes alone (FIG. 23D) or with the proSP-B dimer fraction observed in the absence of liposomes (FIG. 23E).

While proSP-B generates lipoprotein particles, it does not form projection cores with emanating membrane bubbles when reconstituted in an analogous way as SP-BM into liposomes containing either an LB- or ER-like lipid composition (FIG. 25D). Thus, proteolytic processing of proSP-B into mature SP-BM is required to generate the membrane stacks characteristic of LBs.

Lipid-Binding and -Transfer Activity is Required for proSP-B Processing in Mice

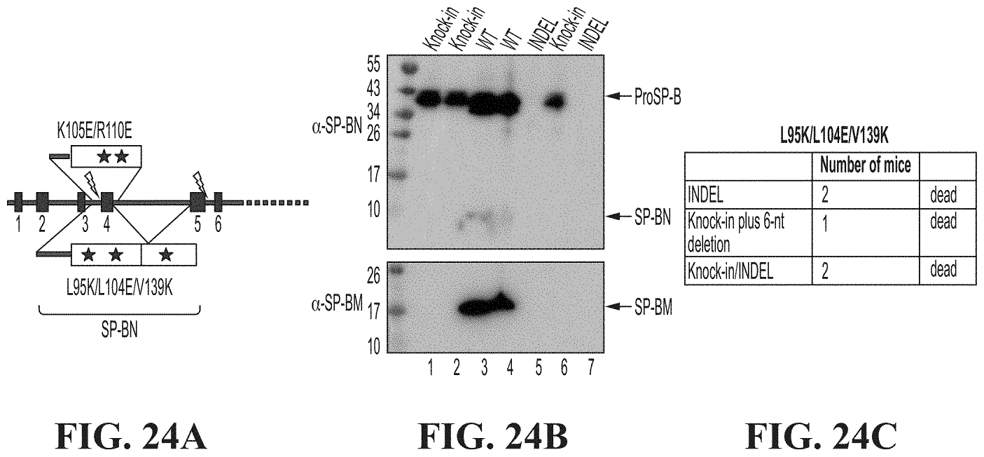

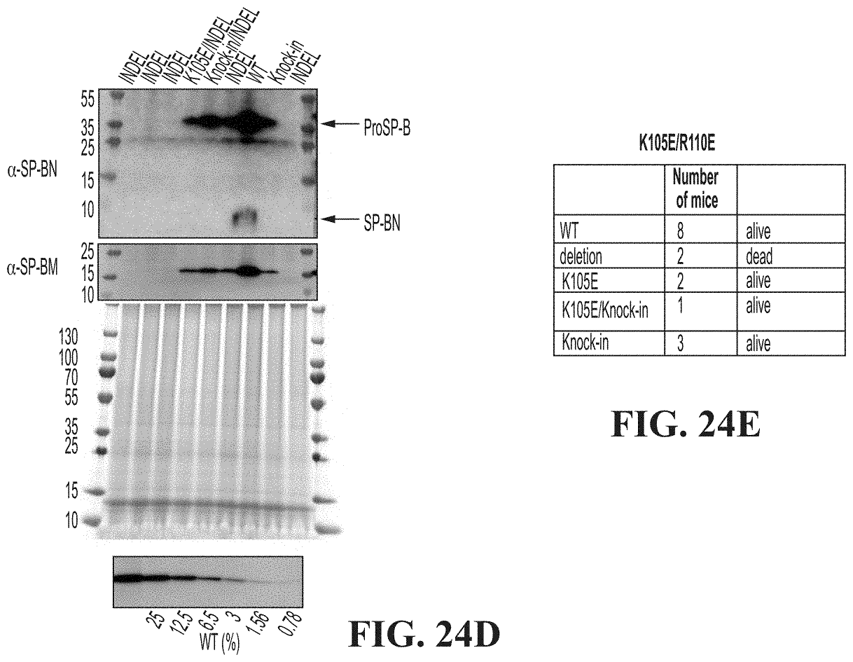

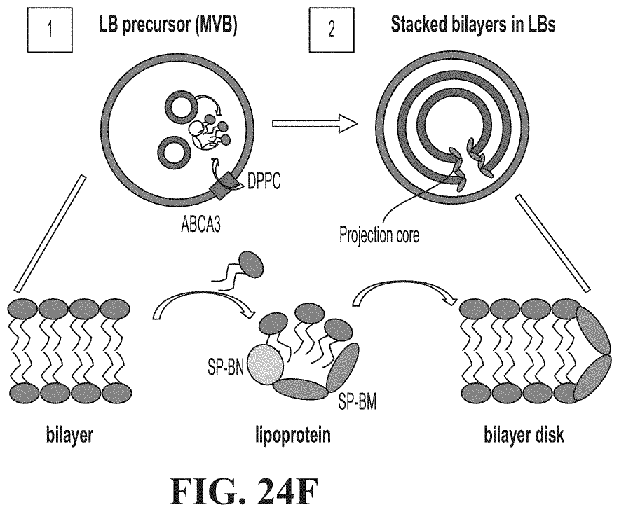

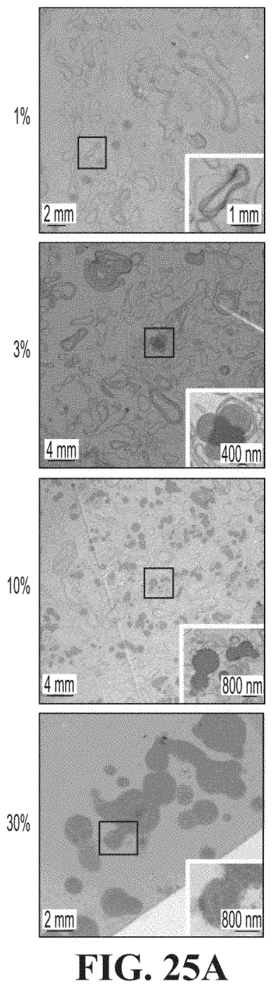

To test the physiological role of SP-B's lipid-binding and -transfer activity, we used CRISPR editing to generate mice carrying mutations in the SP-BN domain. We first generated mice with three mutations in the SP-BN domain (L95K/L104E/V139K, equivalent to L36K/L45E/V80K in SP-BN), which in the isolated SP-BN protein abrogated all lipid binding and caused proSP-B to stay in the ER of MILE-15 cells. Because the mutated residues are located in two different exons with the intervening intron containing a highly repetitive sequence, we used a single-strand DNA donor to knock-in the three point mutations and also remove the intron (FIG. 24A). The embryos were isolated one or two days before birth. Three embryos carried the three mutations in both alleles, as shown by PCR and DNA sequencing. Analysis by SDS-PAGE and immunoblotting showed that all three mice made proSP-B, but no mature SP-BM (FIG. 24B, lanes 1,2,6). As expected, both proSP-B and mature SP-BM were detected in wild-type embryos (lanes 3,4) and these proteins were absent in embryos carrying insertions or deletions in both alleles (lanes 5,7). Thin-section EM showed that the lungs of the triple-mutant embryos did not contain normal LBs (FIG. 31D versus 31C), similar to knock-out embryos (FIG. 31E).

To test for viability, we repeated the CRISPR experiment and analyzed the genotypes of newborn mice (FIG. 24C, Tables 3 and 4). As expected, mice carrying deletions in both alleles of the SP-B gene died shortly after birth because of respiratory failure. Two mice had the desired triple-mutation in one allele and an insertion or deletion in the other (FIG. 24C). Like knock-out mice (Clark et al., 1995), these animals died shortly after birth. Thus, lipid binding to the SP-BN domain is required for the processing of proSP-B and the generation of functional protein in vivo.

Next we used CRISPR editing to generate mice carrying the K105E/R110E mutations, equivalent to K46E/R51E in SP-BN (FIG. 24A). These mutations compromise lipid-transfer, but not-binding, of the isolated SP-BN protein, and reduce proteolytic processing of proSP-B, but not its export from the ER, in ILE-15 cells. We obtained two embryos with the expected knock-in mutations in proSP-B (FIG. 24D). The proteolytic processing of proSP-B was reduced to ˜10% of wild-type levels (FIG. 24D), like in ILE-15 cells. Similar results were obtained with two knock-in mice that contained only one of the two desired mutations (K105E; corresponding to K46E in SP-BN; FIG. 24D). All mutant embryos contained normal LBs (FIGS. 31F; 31G). Accordingly, viable mice were obtained that contained the double-mutation in one allele and a deletion/frame-shift in the other, the single K105E mutation in one allele and a deletion in the other, or the double mutation in one allele and the single K105E mutation in the other (FIG. 24E). We conclude that the low level of proSP-B processing is sufficient to sustain LB formation and viability after birth.

### Discussion of the Results

Provided herein is the mechanism by which SP-B mediates the formation of LBs in alveolar type II cells. The results provided herein show that the targeting of proSP-B to LBs requires lipid-binding and -transfer activity of the SP-BN domain. This is physiologically important, as shown by the effect of mutations in mice. All three saposin-like domains collaborate in the sorting of proSP-B to LBs. In a first step, proSP-B export from the ER requires lipid binding to the SP-BN domain and export is further stimulated by the SP-BC domain. The results show that SP-BN and SP-BC facilitate ER export by recruiting lipids that shield the hydrophobic surface of the SP-BM domain in the ER, preventing its premature interaction with the membrane (FIG. 24F). The structure formed by proSP-B and PLs in the ER might already be a small lipoprotein particle. Its formation would be similar to that of a very-low density lipoprotein (VLDL) particle, where the microsomal transfer protein (MTP) moves triglycerides and other lipids from the ER bilayer to newly synthesized apolipoprotein B (Olofsson et al., 1999; Sirwi and Hussain, 2018). A second sorting step occurs in a low-pH compartment, probably MVBs (FIG. 24F). The low pH activates PL transfer by the SP-BN domain, resulting in the formation of larger lipoprotein particles that are diverted from the secretory pathway. Finally, following proteolytic cleavage of proSP-B, the lipoprotein particles would serve as nucleation sites for the generation of stacked concentric bilayers by the mature SP-BM protein (FIG. 24F). In this model, SP-BN and SP-BM would function together to generate LBs de novo, rather than being sorted to a pre-existing organelle. This model would explain the lack of LBs in SP-B mutants.

The SP-BN domain probably extracts the PLs from the limiting membrane, although some PLs could come from intraluminal vesicles of MVBs (FIG. 24F, upper panel 2). The majority of PLs, particularly the abundant DPPC lipid, are likely transferred into LBs by ABCA3, an ABC transporter in the limiting membrane (Matsumura et al., 2007). In the lipoprotein particles, the acyl chains of the PLs would interact with the hydrophobic face of the SP-BM domain (FIG. 24F). This mechanism is similar to that proposed for the formation of high-density lipoprotein (HDL) particles. Here, the ABC transporter ABCA1 pumps PLs and cholesterol from the inner to the extracellular leaflet of the plasma membrane, and apolipoprotein A1 picks up the lipid and forms a bilayer within the discoidal HDL (Phillips, 2018).

It is contemplated that during the transition from lipoprotein particles to stacked membrane sheets, SP-BM remains in contact with the acyl chains of PLs and eventually localizes to the edges of bilayer discs (FIG. 24F). This would be analogous to the conversion of detergent/lipid/protein micelles into LB-like structures in the in vitro reconstitutions. The postulated localization of SP-BM to bilayer edges is based on properties of the related protein saposin A. Like proSP-B, saposin A forms lipoprotein particles (Li et al., 2016). In a crystal structure with the detergent lauryldimethylamine oxide (LDAO), two saposin A molecules with an open conformation, resembling an open “pocket-knife”, localize to the hydrophobic edges of a small, lipid-bilayer like nanodisc (Popovic et al., 2012). SP-BM may function analogously, as it is predicted to have an extensive hydrophobic surface inside its “pocket-knife” structure (FIG. 33C).

The existence of a protein-rich projection core in human LBs and in our reconstitutions suggests that SP-BM molecules can also self-assemble, causing multiple bilayer disks to be anchored at the same site (FIG. 24F, upper panel 3). In some species, such as mice, projection cores have not been observed and SP-BM is distributed throughout the membrane stacks (Brasch et al., 2004). Assuming that the mechanism of LB formation is conserved, the projection cores in these species might be smaller or they are initially formed and then dissolved. They could be dissolved if membrane sheets can fuse at the site of the projection core to form closed bilayers. SP-BM could then oligomerize to form diffusible pores in the membrane, with the hydrophobic side of their “pocket-knife” structure facing the surrounding lipid, a structure that may also be adopted by the saposin-like, pore-forming proteins granulysin, NK-lysin, and amoebapore (Olmeda et al., 2013). This mechanism is also consistent with the idea that vesicles are generally formed by the bending of bilayer discs and the eventual fusion of their edges (Lasic, 1988). In previous reconstitution experiments, SP-BM formed associated bilayer discs, rather than concentric membrane stacks (Poulain et al., 1992; Suzuki et al., 1989; Williams et al., 1991), perhaps because the protein was not solubilized in octylglucoside before addition of lipids. This observation is consistent with our model assuming that these discs are precursors to membrane stacks and that SP-BM localizes to their edges. Regardless of the exact mechanism of bilayer stacking, it is striking that the internal shape of LBs can be generated with SP-BM alone. The only other organelle that has been reconstituted with purified proteins is the tubular ER network (Powers et al., 2017).

Although SP-BN functions as a domain in proSP-B, our experiments with isolated SP-BN reveal its mechanism of lipid transfer. SP-BN binds PLs in a unique way, as the acyl chains of the centrally bound PL molecule interact exclusively with the lateral PLs and therefore have essentially the same environment as in a lipid bilayer. In most other lipid transfer proteins, the lipid is bound in a proteinaceous pocket or tube (Wong et al., 2019). SP-BN first interacts with negatively charged PL head groups of the bilayer through positively charged amino acids that point outwards from the roof In the next step, the roof opens and allows PL molecules to exit and enter the hollow, with the central molecule likely favored kinetically. When SP-BN is part of proSP-B, it likely transfers PLs in the same way, but the PLs associate with the SP-BM domain in a lipoprotein particle, rather with than another bilayer. This mechanism is different from that of cytosolic lipid transfer proteins, which generally transfer lipids between bilayers of different organelles (Wong et al., 2019). These results do not exclude that processed SP-BN also has a function, as suggested by its anti-microbial activity (Yang et al., 2010).

The exact role of SP-BC domain remains unclear. It is required for efficient ER export of proSP-B, but a small fraction of proSP-B is still proteolytically processed, likely explaining why a transgene lacking SP-BC can rescue SP-B knock-out mice (Akinbi et al., 1997). The crystal structures and biochemical data show that SP-BC does not bind PLs, but a hydrophobic pocket formed in a tetrameric assembly could accommodate a smaller molecule.

LB-like structures can form in cells under pathological or non-physiological conditions in the absence of SP-B, which is exclusively expressed in the lung. For example, many cationic amphiphilic drugs induce phospholipidosis in different cell types, characterized by the accumulation of phospholipids in intra-lysosomal membrane sheets (Breiden and Sandhoff, 2019). Similar structures are also observed in the lysosomes of Tay-Sachs disease patients (Breiden and Sandhoff, 2019) and in tissue culture cells overexpressing ABCA3 (Nagata et al., 2004). Whether all these onion-like structures are generated by a common mechanism remains to be investigated.

These results provide the generation of an improved surfactant mixture that can be administered through the trachea in clinical settings. Commercial surfactant is currently still isolated as a crude mixture from bovine or porcine lungs by extraction with organic solvent. Moreover, whereas it is effective in treating neonatal respiratory distress syndrome in premature infants, it has commercial surfactants have no beneficial effect in ARDS patients (for review, see (Kim and Won, 2018). It is contemplated herein that the commercial mixtures, rather than fully replacing authentic surfactant, simply provide liposomes with non-specifically distorted lipid bilayers, given that mixtures containing only synthetic hydrophobic peptide fragments have a similar effect as those obtained from animal lungs (Hentschel et al., 2020). Animal sources and even synthetic peptides may also not be able to provide the large amounts of surfactant required for the treatment of adult ARDS patients. The improved understanding of the biological functions of the SP-B domains and the ability to produce them recombinantly in E. coli provides a surfactant mixture that can improve outcomes in ARDS.

### REFERENCES

1) Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta Crystallographica. Section D, Biological Crystallography 66, 213-221.
2) Aggarwal, S., Yurlova, L., and Simons, M. (2011). Central nervous system myelin: structure, synthesis and assembly. Trends Cell Biol. 21, 585-593.
3) Ahn, V. E., Faull, K. F., Whitelegge, J. P., Fluharty, A. L., and Privé, G. G. (2003). Crystal structure of saposin B reveals a dimeric shell for lipid binding. Proceedings of the National Academy of Sciences of the United States of America 100, 38-43.
4) Akinbi, H. T., Breslin, J. S., Ikegami, M., Iwamoto, H. S., Clark, J. C., Whitsett, J. A., Jobe, A. H., and Weaver, T. E. (1997). Rescue of SP-B knockout mice with a truncated SP-B proprotein. Function of the C-terminal propeptide. The Journal of Biological Chemistry 272, 9640-9647.
5) Beck, D. C., Ikegami, M., Na, C. L., Zaltash, S., Johansson, J., Whitsett, J. A., and Weaver, T. E. (2000). The role of homodimers in surfactant protein B function in vivo. The Journal of Biological Chemistry 275, 3365-3370.
6) Bernhard, W. (2016). Lung surfactant: Function and composition in the context of development and respiratory physiology. Ann. Anat. 208, 146-150.
7) Bian, X., Klemm, R. W., Liu, T. Y., Zhang, M., Sun, S., Sui, X., Liu, X., Rapoport, T. A., and Hu, J. (2011). Structures of the atlastin GTPase provide insight into homotypic fusion of endoplasmic reticulum membranes. Proceedings of the National Academy of Sciences of the United States of America 108, 3976-3981.
8) BLIGH, E. G., and DYER, W. J. (1959). A rapid method of total lipid extraction and purification. Can J Biochem Physiol 37, 911-917.
9) Brasch, F., Johnen, G., Winn-Brasch, A., Guttentag, S. H., Schmiedl, A., Kapp, N., Suzuki, Y., Müller, K. M., Richter, J., Hawgood, S., et al. (2004). Surfactant protein B in type II pneumocytes and intra-alveolar surfactant forms of human lungs. Am. J. Respir. Cell Mol. Biol. 30, 449-458.
10) Breiden, B., and Sandhoff, K. (2019). Emerging mechanisms of drug-induced phospholipidosis. Biol. Chem. 401, 31-46.
11) Bruhn, H. (2005). A short guided tour through functional and structural features of saposin-like proteins. The Biochemical Journal 389, 249-257.
12) Chander, A., Dodia, C. R., Gil, J., and Fisher, A. B. (1983). Isolation of lamellar bodies from rat granular pneumocytes in primary culture. Biochimica Et Biophysica Acta 753, 119-129.
13) Chander, A., Johnson, R. G., Reicherter, J., and Fisher, A. B. (1986). Lung lamellar bodies maintain an acidic internal pH. The Journal of Biological Chemistry 261, 6126-6131.
14) Chintagari, N. R., Mishra, A., Su, L., Wang, Y., Ayalew, S., Hartson, S. D., and Liu, L. (2010). Vacuolar ATPase regulates surfactant secretion in rat alveolar type II cells by modulating lamellar body calcium. PloS One 5, e9228.
15) Clark, J. C., Wert, S. E., Bachurski, C. J., Stahlman, M. T., Stripp, B. R., Weaver, T. E., and Whitsett, J. A. (1995). Targeted disruption of the surfactant protein B gene disrupts surfactant homeostasis, causing respiratory failure in newborn mice. Proceedings of the National Academy of Sciences of the United States of America 92, 7794-7798.
16) Derman, A. I., Prinz, W. A., Belin, D., and Beckwith, J. (1993). Mutations That Allow Disulfide Bond Formation in the Cytoplasm of Escherichia coli. Science 262, 1744-1747.
17) Emsley, P., and Cowtan, K. (2004). Coot: model-building tools for molecular graphics. Acta Crystallographica. Section D, Biological Crystallography 60, 2126-2132.
18) Evans, P. R., and Murshudov, G. N. (2013). How good are my data and what is the resolution? Acta Crystallographica. Section D, Biological Crystallography 69, 1204-1214.
19) Gerson, K. D., Foster, C. D., Zhang, P., Zhang, Z., Rosenblatt, M. M., and Guttentag, S. H. (2008). Pepsinogen C proteolytic processing of surfactant protein B. The Journal of Biological Chemistry 283, 10330-10338.
20) Hentschel, R., Bohlin, K., van Kaam, A., Fuchs, H., and Danhaive, O. (2020). Surfactant replacement therapy: from biological basis to current clinical practice. Pediatr. Res. 185, 153.
21) Ikegami, M., Whitsett, J. A., Martis, P. C., and Weaver, T. E. (2005). Reversibility of lung inflammation caused by SP-B deficiency. Am. J. Physiol. Lung Cell Mol. Physiol. 289, L962-L970.
22) Kabsch, W. (2010). Xds. Acta Crystallographica. Section D, Biological Crystallography 66, 125-132.
23) Kim, H. C., and Won, Y.-Y. (2018). Clinical, technological, and economic issues associated with developing new lung surfactant therapeutics. Biotechnol. Adv. 36, 1185-1193.
24) King, R. J. (1982). Pulmonary surfactant. J Appl Physiol Respir Environ Exerc Physiol 53, 1-8.
25) Kolter, T., and Sandhoff, K. (2005). Principles of lysosomal membrane digestion: stimulation of sphingolipid degradation by sphingolipid activator proteins and anionic lysosomal lipids. Annu. Rev. Cell Dev. Biol. 21, 81-103.
26) Korimilli, A., Gonzales, L. W., and Guttentag, S. H. (2000). Intracellular localization of processing events in human surfactant protein B biosynthesis. The Journal of Biological Chemistry 275, 8672-8679.
27) Li, J., Richards, M. R., Bagal, D., Campuzano, I. D. G., Kitova, E. N., Xiong, Z. J., Privé, G. G., and Klassen, J. S. (2016). Characterizing the Size and Composition of Saposin A Lipoprotein Picodiscs. Anal. Chem. 88, 9524-9531.
28) Lin, S., Akinbi, H. T., Breslin, J. S., and Weaver, T. E. (1996a). Structural requirements for targeting of surfactant protein B (SP-B) to secretory granules in vitro and in vivo. The Journal of Biological Chemistry 271, 19689-19695.
29) Lin, S., Phillips, K. S., Wilder, M. R., and Weaver, T. E. (1996b). Structural requirements for intracellular transport of pulmonary surfactant protein B (SP-B). Biochimica Et Biophysica Acta 1312, 177-185.
30) Lopez-Rodriguez, E., Gay-Jordi, G., Mucci, A., Lachmann, N., and Serrano-Mollar, A. (2017). Lung surfactant metabolism: early in life, early in disease and target in cell therapy. Cell Tissue Res. 367, 721-735.
31) Mathialagan, N., and Possmayer, F. (1990). Low-molecular-weight hydrophobic proteins from bovine pulmonary surfactant. Biochimica Et Biophysica Acta 1045, 121-127.
32) Matsumura, Y., Sakai, H., Sasaki, M., Ban, N., and Inagaki, N. (2007). ABCA3-mediated choline-phospholipids uptake into intracellular vesicles in A549 cells. FEBS Letters 581, 3139-3144.
33) Melton, K. R., Nesslein, L. L., Ikegami, M., Tichelaar, J. W., Clark, J. C., Whitsett, J. A., and Weaver, T. E. (2003). SP-B deficiency causes respiratory failure in adult mice. Am. J. Physiol. Lung Cell Mol. Physiol. 285, L543-L549.
34) Morin, A., Eisenbraun, B., Key, J., Sanschagrin, P. C., Timony, M. A., Ottaviano, M., and Sliz, P. (2013). Collaboration gets the most out of software. eLife 2, e01456.
35) Nagata, K., Yamamoto, A., Ban, N., Tanaka, A. R., Matsuo, M., Kioka, N., Inagaki, N., and Ueda, K. (2004). Human ABCA3, a product of a responsible gene for abca3 for fatal surfactant deficiency in newborns, exhibits unique ATP hydrolysis activity and generates intracellular multilamellar vesicles. Biochemical and Biophysical Research Communications 324, 262-268.
36) Nogee, L. M., de Mello, D. E., Dehner, L. P., and Colten, H. R. (1993). Brief report: deficiency of pulmonary surfactant protein B in congenital alveolar proteinosis. N. Engl. J. Med. 328, 406-410.
37) Nogee, L. M., Wert, S. E., Proffit, S. A., Hull, W. M., and Whitsett, J. A. (2000). Allelic heterogeneity in hereditary surfactant protein B (SP-B) deficiency. Am. J. Respir. Crit. Care Med. 161, 973-981.
38) Olmeda, B., Garcia-Alvarez, B., and P6rez-Gil, J. (2013). Structure-function correlations of pulmonary surfactant protein SP-B and the saposin-like family of proteins. European Biophysics Journal 42, 209-222.
39) Olofsson, S. O., Asp, L., and Boren, J. (1999). The assembly and secretion of apolipoprotein B-containing lipoproteins. Curr. Opin. Lipidol. 10, 341-346.
40) Phillips, M. C. (2018). Is ABCA1 a lipid transfer protein? J. Lipid Res. 59, 749-763.
41) Popovic, K., Holyoake, J., Pomes, R., and Privé, G. G. (2012). Structure of saposin A lipoprotein discs. Proceedings of the National Academy of Sciences of the United States of America 109, 2908-2912.
42) Powers, R. E., Wang, S., Liu, T. Y., and Rapoport, T. A. (2017). Reconstitution of the tubular endoplasmic reticulum network with purified components. Nature 543, 257-260.
43) Rabouille, C., and Linstedt, A. D. (2016). GRASP: A Multitasking Tether. Front Cell Dev Biol 4, 1.
44) Ridsdale, R., Na, C.-L., Xu, Y., Greis, K. D., and Weaver, T. (2011). Comparative proteomic analysis of lung lamellar bodies and lysosome-related organelles. PloS One 6, e16482.
45) Rodriguez, D. D., Grosse, C., Himmel, S., Gonzalez, C., de Ilarduya, I. M., Becker, S., Sheldrick, G. M., and Usón, I. (2009). Crystallographic ab initio protein structure solution below atomic resolution. Nat. Methods 6, 651-653.
46) Sirwi, A., and Hussain, M. M. (2018). Lipid transfer proteins in the assembly of apoB-containing lipoproteins. J. Lipid Res. 59, 1094-1102.
47) Stahlman, M. T., Gray, M. P., Falconieri, M. W., Whitsett, J. A., and Weaver, T. E. (2000). Lamellar body formation in normal and surfactant protein B-deficient fetal mice. Lab. Invest. 80, 395-403.
48) Stratton, C. J. (1978). The ultrastructure of multilamellar bodies and surfactant in the human lung. Cell Tissue Res. 193, 219-229.
49) Terwilliger, T. C., Grosse-Kunstleve, R. W., Afonine, P. V., Moriarty, N. W., Zwart, P. H., Hung, L. W., Read, R. J., and Adams, P. D. (2008). Iterative model building, structure refinement and density modification with the PHENIX AutoBuild wizard. Acta Crystallographica. Section D, Biological Crystallography 64, 61-69.
50) Ueno, T., Linder, S., Na, C.-L., Rice, W. R., Johansson, J., and Weaver, T. E. (2004). Processing of pulmonary surfactant protein B by napsin and cathepsin H. The Journal of Biological Chemistry 279, 16178-16184.
51) Vaccaro, A. M., Salvioli, R., Tatti, M., and Ciaffoni, F. (1999). Saposins and their interaction with lipids. Neurochem. Res. 24, 307-314.
52) Wikenheiser, K. A., Vorbroker, D. K., Rice, W. R., Clark, J. C., Bachurski, C. J., Oie, H. K., and Whitsett, J. A. (1993). Production of immortalized distal respiratory epithelial cell lines from surfactant protein C/simian virus 40 large tumor antigen transgenic mice. Proceedings of the National Academy of Sciences of the United States of America 90, 11029-11033.
53) Williams, M. C., Hawgood, S., and Hamilton, R. L. (1991). Changes in lipid structure produced by surfactant proteins SP-A, SP-B, and SP-C. Am. J. Respir. Cell Mol. Biol. 5, 41-50.
54) Wong, L. H., Gatta, A. T., and Levine, T. P. (2019). Lipid transfer proteins: the lipid commute via shuttles, bridges and tubes. Nat. Rev. Mol. Cell Biol. 20, 85-101.
55) Xu, Y., Wang, S., Hu, Q., Gao, S., Ma, X., Zhang, W., Shen, Y., Chen, F., Lai, L., and Pei, J. (2018). CavityPlus: a web server for protein cavity detection with pharmacophore modelling, allosteric site identification and covalent ligand binding ability prediction. Nucleic Acids Research 46, W374-W379.
56) Yang, L., Johansson, J., Ridsdale, R., Willander, H., Fitzen, M., Akinbi, H. T., and Weaver, T. E. (2010). Surfactant protein B propeptide contains a saposin-like protein domain with antimicrobial activity at low pH. J. Immunol. 184, 975-983.
57) Zheng, H., Cooper, D. R., Porebski, P. J., Shabalin, I. G., Handing, K. B., and Minor, W. (2017). CheckMyMetal: a macromolecular metal-binding validation tool. Acta Crystallogr. D. Struct. Biol. 73, 223-233.

### Example 3: Experimental Model and Subject Details

### Tissue Culture Cells

Murine lung epithelial (MLE-15) and HEK293T cells are maintained in liquid nitrogen and early passage aliquots are thawed periodically. MLE-15 cells are maintained in HITES medium supplemented with 2% fetal bovine serum (FBS) at 37° C. (Wikenheiser et al., 1993). HEK293T cells are maintained in DMEM supplemented with 10% FBS at 37° C.

### Method Details

### Cloning

Cloning of sgRNAs into pX330 was performed following a modified single-step digestion-ligation protocol available on the world wide web at genome-engineering.org. Synthetic SP-BN and SP-BC codon-optimized for E. coli were cloned into pET-32a(+) using BamH and EagI sites. Human proSP-B lacking the SP-BC domain (amino acids 1-279) with a C-terminal Flag tag was cloned into pcDNA3.1+ using NEBuilder HiFi DNA Assembly (NEB). Human proSP-B lacking both SP-BM and SP-BC domains (SP-ΔMC; amino acids 1-200) was cloned into pCS6 using EcoRI and HindIII sites. Human proSP-B (24-381) was cloned into the lentiviral pHAGE2 vector using EcoRI and NotI sites. Human proSP-B lacking the signal sequence and the SP-BC domain (amino acids 24-279) was cloned into pGEX-6P-1 or pHAGE2, and a thrombin cleavage site (LVPRGS) was introduced between residues 201-202 by QuikChange Site-Directed Mutagenesis Kit (Agilent). All point mutations were introduced by QuikChange Site-Directed Mutagenesis (Agilent) and verified by DNA sequencing Protein purifications

SP-BN and SP-BC were expressed with N-terminal His₆ and thioredoxin tags, followed by a Tobacco Etch virus (TEV) protease cleavage site (ENLYFQS, SEQ ID NO: 5). The fusion proteins were expressed in E. coli cells lacking thioredoxin reductase (Rosetta-Gami 2 (DE3) cells) from an isopropyl P-D-1-thiogalactopyranoside (IPTG)-inducible promoter of the pET-32a(+) plasmid. The cells were grown to mid log phase in the presence of 100 μg/ml ampicillin and induced with 1 mM IPTG. Cell pellets from a 6 L culture were homogenized in −100 ml 20 mM Tris/HCl, pH 8, 300 mM NaCl, 20 mM imidazole, 10% glycerol and protease inhibitors. The extract was centrifuged for 45 min at 40,000 g in a Beckman Ti45 rotor. The supernatant was incubated with 5 ml Ni-NTA beads for 1 hr, and the protein was eluted with 300 mM imidazole in the same buffer. The sample was supplemented with 5 mM glutathione (GSH)/0.5 mM oxidized glutathione (GSSG) and 0.05-0.1 mg/ml TEV protease and dialyzed overnight against 20 mM Tris/HCl, pH 8, 200 mM NaCl, 5% glycerol, 5 mM GSH, 0.5 mM GSSG using a membrane with a molecular weight cut-off of 3.5 kDa. The sample was incubated with −1 ml Ni-NTA resin to remove the tags, concentrated, and applied to a Superdex 75 Increase column using the same buffer without GSH and GSSG. Peak fractions were concentrated and used for assays or crystallization. All SP-BN mutant proteins were purified as described above.

SP-BM was purified from bovine lungs as follows. Lungs from one bovine calf were cut into small pieces and passed twice through a household mincer. Organic extraction was performed according to (BLIGH and DYER, 1959). The organic phase was dried using a rotary evaporator and dissolved in −15 ml chloroform/methanol/0.1 M HCl (1:1:0.05) and loaded on a Sephadex LH-20 column equilibrated with the same solvent. Fractions that contain SP-BM based on Western blotting were pooled, dried using a rotary evaporator and solubilized in 20 mM Tris, pH 8, 200 mM NaCl, 2% glycerol, 6% n-octyl-o-D-glucopyranoside (OG) for 1 h. After centrifugation at 10,000 g for 10 min, the supernatant was applied to a Superdex 200 column using the same buffer except with 1.5% OG. Fractions containing SP-BM were pooled and concentrated. Recombinant SP-BM was purified as follows. A fusion construct containing N-terminal His6 and GST tags and human SP-BN and SP-BM domains with a thrombin cleavage site in between was expressed in E. coli cells (BL21 (DE3)) from an IPTG inducible promoter of the pGEX-6P-1 plasmid. The cells were grown in 2×YT medium at 37° C. to mid log phase and induced with 1 mM IPTG at 30° C. overnight. Cell pellets from a 6 L culture were homogenized at 25,000 psi (Avestin Emulsiflex-C3) in 150 ml 20 mM Tris/HCl pH 8, 200 mM NaCl and protease inhibitors. The sample was centrifuged for 10 min at 15,000 g at 4° C. (Sorvall RC5C Plus centrifuge, SLA-600TC rotor) to sediment the inclusion bodies. The pellet was resuspended and washed twice in 150 mL 20 mM Tris/HCl pH 8, 200 mM NaCl, 1% Triton. The pellet was resuspended in 50 mL 20 mM Tris/HCl pH 8, 200 mM NaCl, 0.6% sarkosyl and homogenized at 25,000 psi in an Emulsiflex instrument. The sample was incubated at room temperature for 1 hr and spun for 10 min at 15,000 g at 4° C. to remove any insoluble material. 2,000 U of thrombin (Thomas Scientific Inc.) was added to the supernatant and the sample was dialyzed against 2 L of 20 mM Tris/HCl pH 8.0, 200 mM NaCl for 4 hrs at room temperature using a membrane with a cut-off of 3.5 kDa. The solution complemented with 20 mM 13-mercaptoethanol (0-Me) and incubated for 30 min at room temperature. To extract the SP-BM, 190 mL chloroform:methanol (1:2 (v/v)) was added and shaken for 1 min followed by incubation for 1 hr at room temperature. Additional 64 mL chloroform were added, shaken for 1 min, then 64 mL of buffer (20 mM Tris/HCl pH 8.0, 200 mM NaCl, 20 mM 13-Me, 0.1% sarkosyl) for 1 min. The organic phase was collected and the aqueous phase was re-extracted with 90 mL chloroform. The combined organic phase was reduced to ˜8 mL with a Rotavapor (Buchi R-114) and applied to a Sephadex LH-20 size exclusion column equilibrated in chloroform:methanol:0.1M HCl (1:1:0.05 (v/v/v)). Fractions containing the protein were combined and the organic solvent was removed with a Rotavapor. Protein was solubilized in 20 mM Tris/HCl pH 8.0, 200 mM NaCl, 6% 13-octylglucoside (Anatrace) and subjected to a Superdex200 Increase 3.2/300 GL column (GE Healthcare Life Sciences) equilibrated in 20 mM Tris/HCl pH 8.0, 200 mM NaCl, 1.5% 13-octylglucoside. The peak fractions were concentrated using a membrane with a 3.5 kDa cut-off.

To purify proSP-B, the protein was expressed as a fusion with an N-terminal HALO tag. The gene was cloned into the lentiviral pHAGE2-DN-CMV-FLAG-HT7-PreScission vector and HEK293T cells were infected with lentiviral particles. Stable cell lines were selected by growth in the presence of 10 μg/ml blasticidin. The cells were grown in 15-cm plates until confluent, after which the medium was replaced with Opti-MEM I (Thermo Fisher) and collected every day for 3-4 days. About 400 ml of medium was incubated with 0.5 ml HALO resin (Promega) at room temperature for 4 hrs. The beads were washed with 20 mM Tris pH 8, 200 mM NaCl, 5% glycerol. They were resuspended in 0.5 ml buffer and incubated overnight at 4° C. with 20 μg/ml of a fusion between GST and 3C protease (GST-3C). The supernatant was incubated with 100 tl GSH beads to remove the protease and concentrated with a 30 kDa cut-off filter.

### Lipid Transfer Assays

To measure lipid transfer by flotation, liposomes containing an ER-like PL composition (65% 1-palmitoyl-2-oleoyl-sn-glycero-3-phosphocholine (POPC), 15% 1,2-dioleoyl-sn-glycero-3-phospho-L-serine (DOPS), 17% 1,2-dioleoyl-sn-glycero-3-phosphoethanolamine (DOPE)) and 1.5% fluorescent NBD-PE at 0.9 mM final concentration of total lipids were incubated with 0.4 mM purified SP-BN in 50 tl of 0.1 M citrate buffer pH 5.4, 200 mM NaCl for 1 h at 370 C. The sample was mixed with an equal volume of 80% Nycodenz in the same buffer, and overlayed with 50 tl 30% Nycodenz, 30 tl 15% Nycodenz and 30 tl buffer. The sample was centrifuged in a TLS55 rotor (Beckman) at 48,000 rpm for 1 h at 4° C. Fractions of 40 tl were taken from the top and analyzed in a fluorescence plate reader (FlexStation 3, Molecular Devices). An aliquot was analyzed by SDS-PAGE and Coomassie-blue staining.

To measure lipid transfer by fluorescence de-quenching, liposomes contained an ER-like PL composition (see above) or an LB-like composition (70% dipalmitoyl phosphatidylcholine (DPPC), 30% phosphatidylglycerol (PG)). Donor liposomes also contained 1.5% NBD-PE and 1.5% rhodamine-PE. The assay was performed in 0.1 M citrate buffer pH 5.4, 200 mM NaCl or 0.1 M Tris buffer pH 7.4 or 7.5, 200 mM NaCl and contained in 50 tl 0.2 mM total lipid of donor vesicles, 0.6 mM lipid of acceptor vesicles, and different concentrations of SP-BN or proSP-B. The samples were incubated at 37° C. in a fluorescence plate reader (excitation 460 nm; emission 538 nm). All readings were normalized to fluorescence measured after addition of 0.5% Triton X-100.

To determine lipid transfer by the filtration assay, liposomes of ER- or LB-like PL composition (final lipid concentration 1 mM) were incubated with SP-BN (final concentration 0.1 mM) in 100 tl final volume for different time periods at 37° C. The samples were placed on ice and centrifuged in an Eppendorf centrifuge at 14,000×g through 100 kDa molecular weight cut-off filters (Amicon Ultra 0.5 ml). Forty tl of the filtrate was extracted with chloroform/methanol, the organic phase was dried, and the pellet resuspended in 10 tl chloroform. TLC was performed with silica plates and 60% chloroform/35% methanol/5% H2O as solvent. Lipids were stained with Primuline and visualized in a fluorescence scanner.

Reconstitution of SP-BM into Liposomes

A mixture of 100 tg DPPC+50 tg egg PG in chloroform was evaporated to dryness under nitrogen and then in vacuum overnight. To the dried residue, 15 tg SP-BM was added, and the volume was brought to 100 tl with SEC buffer (20 mM Tris, pH 8.0, 200 mM NaCl, 2% glycerol, 1.5% OG). The mixture was incubated at 40° C. for 1 h and then diluted 5 times with 20 mM Tris, pH 7.4, 200 mM NaCl, 1 mM EDTA, and dialyzed against 2-5 changes of the same buffer. 100 tl aliquots were then incubated overnight at 37° C. with or without CaCl₂) (final concentration of 5 mM).

### Thin-Section EM

Reconstituted samples were fixed in suspension by adding an equal volume of fixative (2.5% paraformaldehyde, 5% glutaraldehyde, 0.06% picric acid in 0.2 M cacodylate buffer). After 1 h at room temperature, the samples were spun at 10,000-20,000×g for 15 min and stored overnight in fixative at 4° C. The samples were washed in 0.1 M cacodylate buffer and postfixed with 1% osmium tetroxide/1.5% potassium ferrocyanide for 1 h, washed in water 3 times and incubated in 1% aqueous uranyl acetate for 1 h, followed by 2 washes in water and subsequent dehydration in grades of alcohol (10 min each in 50%, 70%, 90%; and 2 times 10 min in 100%). The samples were then put in propylene oxide for 1 h and infiltrated overnight in a 1:1 mixture of propylene oxide and TAAB Epon (Marivac Canada Inc. St. Laurent, Canada). The following day the samples were embedded in TAAB Epon and polymerized at 60° C. for 48 h.

Ultrathin sections (about 60 nm) were cut on a Reichert Ultracut-S microtome, picked up on to copper grids stained with lead citrate and examined in a TecnaiG2 Spirit BioTWIN and images were recorded with an AMT 2k CCD camera.

### Negative-Stain EM

Formvar/Carbon 400 mesh copper grids (Ted Pella) were plasma cleaned in EMS100X for 30 s at 30 mA. The grids were incubated with 3 μl sample of SEC fractions for 30 s. Excess solution was blotted away and the grids were stained with 1.5% uranyl formate for 30 s. Micrographs were acquired on a Phillips CM10 microscope equipped with a Gatan UltraScan 894 2k CCD camera and operated at 100 kV. Images were acquired at a magnification of 52,000.

Experiments with MLE-15 Cells

To generate SP-B knock-out ILE-15 cells, 1,160,250 cells were plated on a 60-mm dish and transfected the next day with 2.5 μg each of two sgRNA-expressing plasmids and 0.5 μg of an EYFP plasmid that confers hygromycin resistance, using Lipofectamine 3000 (Invitrogen). One day after transfection, cells were split 1:5 and media supplemented with 300 μg/ml hygromycin were added every other day to select for positive clones. Ten days after begin of the hygromycin treatment, the transfected and selected cells were trypsinized and diluted to achieve 1 cell per 100 μl for seeding in 96-well plates. Single-cell wells were progressively expanded in size up to 6-well plates, when they were split into two, so that half of the cells could be used for PCR and sequencing of genomic DNA. For transient expression of various SP-B constructs, 525,000 cells were plated per well in a 6-well plate and transfected the next day with 2.5 μg of total plasmid using Lipofectamine 3000. Two days after transfection, cells were scraped in medium and centrifuged at 1000×g for 5 min. Medium was saved, cells were washed with PBS and resuspended in 500 μl 20 mM Tris/HCl, pH8, 5 mM EDTA and protease inhibitors. After passing through a 22G needle 25 times, samples were centrifuged again at 1000×g for 5 min. The post-nuclear supernatant (PNS) was centrifuged at 16,000×g for 15 min to obtain the cellular membranes, which were resuspended in PBS. In some experiments, the resuspended membranes were mixed with an equal volume of 0.2 M Na2CO3 (pH >11) or TX-100 was added to a final concentration of 1%. After 30-min incubation on ice, insoluble material was sedimented by centrifugation at 16,000×g for 30 min and washed with PBS. For immunoblotting human proSP-B with mouse F-2 mAb, samples were reduced by adding P-mercaptoethanol to the SDS-loading buffer. Immunoblots using rabbit anti-mature SP-B and rabbit anti-mouse SP-BN (for detecting both mouse SP-BN and mouse proSP-B) were done under non-reducing conditions. Immunoblots were visualized with an Amersham Imager 600.

### Lamellar Body Enrichment

Lamellar bodies were isolated by modifications of the method of (Chander et al., 1983) and (Matsumura et al., 2007). Confluent cells (five 100-mm dishes) were harvested and sonicated in PBS containing 1 M sucrose and a protease inhibitor mixture using a probe sonicator. Sonication was performed with two bursts of 15 s each with a 5 s interval and a 40% maximum power output. The sample was then centrifuged at 1,000 g for 10 min to obtain a post-nuclear supernatant (PNS). A sucrose gradient consisting of 1 ml each of 0.8, 0.7, 0.6, 0.5, 0.4, 0.3 M, and 0.2 M sucrose was layered successively above 3.5 ml of PNS. The gradient was spun in a Beckman SW 55 Ti rotor at 80 000 g for 3 h. After centrifugation, 1 ml of each fraction was collected from the top.

### Immunostaining of MLE-15 Cells

For immunofluorescence, 52,500 SP-B KO ILE-15 cells per well were set up in a 8-well chamber slide and transfected the next day with 0.25 □g of human proSP-B (wild type or L96K/L105E/L140K mutant) using Lipofectamine 3000. Two days after transfection, cells were fixed with 4% formaldehyde, permeabilized with 0.2% saponin and stained with mouse anti-human SP-B (F-2) and rabbit anti-mature SP-B antibodies.

Experiments with Mice

A cocktail of 0.61 pmol/μl of an equimolar mixture of crRNA and tracrRNA, 100 ng/μl Cas9 protein (Aida et al, 2015) and 10 ng/l single-stranded oligodeoxynucleotide (ssODN, IDT) was injected into the pronuclei of E0.5 embryos (C57BL6/Envigo). Post-injection surviving embryos were re-implanted into recipient CD1 pseudo-pregnant females and either allowed to develop to term or harvested at E18.5. For immunoblotting, lungs were cut into small pieces, placed in 1 ml RIPA buffer with protease inhibitors, and homogenized in a bead beater using an equal volume of glass beads. Lysates were cleared by centrifugation first at 1000×g and then at 10,000×g. For electron microscopy, lung pieces were placed in EM fixative and processed as above.

### Lipid Analysis by Mass Spectrometry

Lipids were extracted by adding 50% acetonitrile to purified proteins and analyzed using a Thermo Q Exactive Plus mass spectrometer coupled to a Thermo Ultimate 3000 uHPLC. Lipids were identified using Thermo Lipidsearch 4.1.

### CD Spectroscopy

The CD spectra were acquired on a Jasco J-815 spectropolarimeter equipped with a PFD-425S/15 peletier unit. Spectra were accumulated 5 times from 260-200 nm, using a scanning rate of 50 nm/min. Measurements were performed at 20° C. in a 1 mm Quartz cuvette. Molecular ellipticities were calculated for the mean residue weight (MRW). Protein concentrations were 0.1-0.3 mg/ml.

Enrichment of lipoprotein particles by SEC: ProSP-B (20 μg), purified as above, was mixed with 1.2 mM liposomes containing a LB-like lipid composition (70% DPPC, 30% egg PG) and 1.5% fluorescent NBD-PE and incubated in either 100 mM citrate pH 5.6, 200 mM NaCl or 20 mM Tris/HCl pH 7.5, 200 mM NaCl for 30 min at 37° C. or 0° C. The samples were then applied to a Superose 6 Increase 3.2/300 GL column equilibrated in either pH 5.6 or pH 7.5 buffer. Absorbance was monitored at both 280 nm and 560 nm. Fractions were collected and analyzed by SDS-PAGE and Western blotting.

### Crystallization

Crystal conditions were screened using the hanging drop method in a 96 well format with a Mosquito robot (TTP Labtech). Crystallization trays were incubated at 20° C. with the exception of trays for SP-BN K46E/R51E, which were incubated at 14° C. In all cases, crystals appeared in 1-3 days.

Crystals of wild-type SP-BN were obtained by mixing the protein solution (7-12 mg/mL 1:1 with mother liquor consisting of 100 mM citrate buffer pH 5.4, 150 mM sodium chloride, 17% (w/v) PEG 3,350. Crystals were incubated briefly in cryoprotection solution consisting of mother liquor with 25% glycerol before flash freezing in liquid nitrogen.

Crystals of SP-BN Y59A/H79Δ were obtained by mixing the protein solution (62 mg/mL) 1:1 with mother liquor consisting of 100 mM sodium/potassium phosphate pH 6.2, 200 mM sodium chloride, 47.5% (w/v) PEG 200. Crystals were flash frozen in liquid nitrogen without additional cryoprotection.

Crystals of SP-BN K46E/R51E were obtained by mixing the protein solution (49 mg/mL) 1:1 with mother liquor consisting of 100 mM citrate buffer pH 5.4, 23-25% (w/v) PEG 4,000, 14-15% 2-propanol. Crystals were incubated briefly in cryoprotection solution consisting of 100 mM citrate buffer pH 5.4, 26% (w/v) PEG 4,000, 25% glycerol before flash freezing in liquid nitrogen.

Crystals of SP-BC at high pH were obtained by mixing the protein solution (25 mg/mL) 1:1 with mother liquor consisting of 100 mM Tris pH 8.5, 200 mM lithium sulfate, 1,260 mM ammonium sulfate. Crystals were harvested directly from the 96-well screen, incubated briefly in cryoprotection solution consisting of mother liquor with 25% glycerol, and flash frozen in liquid nitrogen.

Crystals of SP-BC at low pH were obtained by mixing the protein solution (25 mg/mL) 1:1 with mother liquor consisting of 170 mM ammonium sulfate, 21-23% PEG 4,000, 15% glycerol. The pH of the crystallization solution was found to be 4.5. Crystals were incubated briefly in cryoprotection solution consisting of mother liquor with 25% glycerol before flash freezing in liquid nitrogen.

### Structure Determination

Data were collected on beamlines 24-ID-C and 24-ID-E at the Advanced Photon Source (Argonne National Laboratory). Data were processed with XDS (Kabsch, 2010) and analyzed with Aimless (Evans and Murshudov, 2013). Datasets have been deposited at SBgrid (Morin et al., 2013).

Data for wild-type SP-BN were collected at a wavelength of 1.7712 Å. The crystals belonged to space group P212121. Seven datasets collected from three crystals (DOIS: doi:10.15785/SBGRID/757, doi:10.15785/SBGRID/758, doi:10.15785/SBGRID/759, doi:10.15785/SBGRID/760, doi:10.15785/SBGRID/761, doi:10.15785/SBGRID/762, doi: 10.15785/SBGRID/763) were combined using the scale-and-merge program within Phenix (Adams et al., 2010) to produce a merged dataset with a resolution of 2.2 Δ and an anomalous completeness of 95.21%. A thorough sub-structure search yielded initial phases using single-wavelength anomalous dispersion of sulfur atoms with Phenix Autosol (Adams et al., 2010). An initial structure was obtained with Phenix AutoBuild (Terwilliger et al., 2008), and the structure was completed with iterative rounds of manual adjustment in Coot (Emsley and Cowtan, 2004) and refinement in Phenix (Adams et al., 2010). TLS parameters were enabled toward the end of refinement, and a riding hydrogen model was employed. Lipid densities were modeled as phosphatidylethanolamine (16:1/18:1), the most abundant species identified by mass spectrometry of the protein sample. The central lipids were modeled with two alternative conformations (i.e., with the head groups at either end of the central cavity), each with an occupancy of 0.5.

Data for SP-BN Y59A/H79A were collected at a wavelength of 0.9791 Å. The crystals belonged to space group C121. Data used for structure determination (doi: 10.15785/SBGRID/773) were collected from a single crystal, which diffracted to 2.3 Å. Initial phases were obtained by molecular replacement, searching for four copies of a single dimer of wild-type SP-BN. Lipids were removed from the search model to avoid biasing the resulting solution. An initial structure was obtained with Phenix AutoBuild (Terwilliger et al., 2008), and the structure was completed with iterative rounds of manual adjustment in Coot (Emsley and Cowtan, 2004) and refinement in Phenix. TLS parameters were enabled toward the end of refinement, and a riding hydrogen model was employed. Lipid densities were modeled as phosphatidylethanolamine (18:0/18:0), with some acyl chains and head groups truncated during manual adjustment owing to poor density, which was likely reflective of the increased heterogeneity of the bound lipids in this structure.

Data for SP-BN K46E/R51E were collected at a wavelength of 0.9792 Å. The crystals belonged to space group P1211. Four datasets collected from one crystal (DOIs: doi: 10.15785/SBGRID/768, doi: 10.15785/SBGRID/769, doi: 10.15785/SBGRID/770, doi: 10.15785/SBGRID/771) were combined to form a merged dataset with a resolution of 1.88 Å. Initial phases were obtained by molecular replacement, searching for two copies of a single dimer of wild-type SP-BN. Lipids were removed from the search model to avoid biasing the resulting solution. An initial structure was obtained with Phenix AutoBuild (Terwilliger et al., 2008), and the structure was completed with iterative rounds of manual adjustment in Coot (Emsley and Cowtan, 2004) and refinement in Phenix. TLS parameters were enabled toward the end of refinement, and a riding hydrogen model was employed. Lipid densities were modeled as phosphatidylethanolamine (16:1/18:1). The central lipids were modeled with two alternative conformations (i.e., with the head groups at either end of the central cavity). Given the higher resolution of this dataset, alternative conformation occupancies were refined.

Data for SP-BC at high pH were collected at a wavelength of 0.9792 Å. The crystals belonged to space group P212121. Data used for structure determination (doi: 10.15785/SBGRID/766) were collected from a single crystal, which diffracted to 1.75 Å. A solution was obtained using the ab initio phasing software ARCIMBOLDO (Rodriguez et al., 2009), using a search for four helical fragments of fourteen residues each. An initial structure was obtained with Phenix AutoBuild (Terwilliger et al., 2008), and the structure was completed with iterative rounds of manual adjustment in Coot (Emsley and Cowtan, 2004) and refinement in Phenix. A metal ion was observed to participate in crystal contacts and was assigned as zinc in a trigonal bipyramidal configuration coordinated by two histidines, two aspartates, and a water molecule; this assignment was validated with the program CheckMyMetal (Zheng et al., 2017).

Data for SP-BC at low pH were collected at a wavelength of 0.9791 Å. The crystals belonged to space group P1211. Data used for structure determination (doi:10.15785/SBGRID/767) were collected from a single crystal, which diffracted to 1.90 Å. Initial phases were obtained by molecular replacement, searching for eight copies of the two helices of SP-BC at high pH that are connected by a single disulfide bond (i.e., residues A26 to V45 of the C terminal domain). An initial structure was obtained with Phenix AutoBuild (Terwilliger et al., 2008), and the structure was completed with iterative rounds of manual adjustment in Coot (Emsley and Cowtan, 2004) and refinement in Phenix. Three sulfate molecules were placed into strong difference density due to the presence of this ion in the crystallization solution and plausible coordinating residues. The program PISA predicts that the protein forms a tetramer.

### Quantification and Statistical Analysis

All experiments were performed at least three times. The figures show representative experiments.

### Structure Validation Report

TABLE 1
Data collection and refinement statistics.

| | | | SP-BN | | SP-BN | |
|---|---|---|---|---|---|---|
| | SP-BN | | (Y59A/H79A) | | (K46E/R51E) | |
| Wavelength | 1.771 | | 0.979 | | 0.979 | |
| Resolution range | 70.82-2.2 | (2.279-2.2) | 66.46-2.315 | (2.397-2.315) | 59.13-1.88 | (1.947-1.88) |
| Space group | P 21 21 21 | | C 1 2 1 | | P 1 21 1 | |
| Unit cell | 93.707 | | 90.335 | | 49.793 | |
| | 108.156 | | 169.527 | | 66.124 | |
| | 114.741 90 | | 73.714 90 | | 59.62 90 | |
| | 90 90 | | 126.897 90 | | 97.37 90 | |
| Total reflections | 1804081 | (66076) | 263140 | (20487) | 1681104 | (111407) |
| Unique reflections | 112655 | (7105) | 37983 | (3077) | 31237 | (2186) |
| Multiplicity | 16.0 | (6.6) | 6.9 | (6.0) | 53.8 | (36.2) |
| Completeness (%) | 95.15 | (61.16) | 97.45 | (80.13) | 92.19 | (70.58) |
| Mean I/sigma(I) | 24.77 | (0.42) | 13.34 | (0.72) | 40.86 | (2.41) |
| Wilson B-factor | 56.50 | | 77.25 | | 18.87 | |
| R-merge | 0.2901 | (1.905) | 0.08439 | (2.03) | 0.1001 | (1.511) |
| R-meas | 0.2986 | (2.065) | 0.09118 | (2.21) | 0.101 | (1.531) |
| R-pim | 0.06995 | (0.7777) | 0.03419 | (0.8571) | 0.01347 | (0.2406) |
| CC1/2 | 0.994 | (0.236) | 0.995 | (0.518) | 1 | (0.897) |
| CC* | 0.998 | (0.618) | 0.999 | (0.826) | 1 | (0.972) |
| Reflections | 108835 | (7019) | 37541 | (3077) | 28887 | (2186) |
| used in | | | | | | |
| refinement | | | | | | |
| Reflections | 3748 | (230) | 1826 | (156) | 1840 | (133) |
| used for | | | | | | |
| R-free | | | | | | |
| R-work | 0.2237 | (0.4367) | 0.2411 | (0.3947) | 0.1695 | (0.2980) |
| R-free | 0.2380 | (0.4878) | 0.2620 | (0.3926) | 0.2171 | (0.3505) |
| CC(work) | 0.943 | (0.451) | 0.945 | (0.591) | 0.907 | (0.547) |
| CC(free) | 0.935 | (0.453) | 0.967 | (0.530) | 0.874 | (0.340) |

| | | | |
|---|---|---|---|
| Number of | 6069 | 5626 | 3318 |
| non-hydrogen | | | |
| atoms | | | |
| Macromolecules | 5098 | 4992 | 2608 |
| Ligands | 784 | 603 | 392 |
| Solvent | 187 | 31 | 318 |
| Protein residues | 628 | 629 | 320 |
| RMS(bonds) | 0.003 | 0.002 | 0.006 |
| RMS(angles) | 0.69 | 0.64 | 0.70 |
| Ramachandran | 99.02 | 95.43 | 100.00 |
| favored (%) | | | |
| Ramachandran | 0.82 | 4.24 | 0.00 |
| allowed (%) | | | |
| Ramachandran | 0.16 | 0.33 | 0.00 |
| outliers (%) | | | |
| Rotamer outliers | 0.34 | 1.04 | 0.65 |
| (%) | | | |
| Clashscore | 2.53 | 3.86 | 1.61 |
| Average B-factor | 84.04 | 106.28 | 35.02 |
| Macromolecules | 83.22 | 103.82 | 30.63 |
| Ligands | 93.30 | 127.48 | 60.58 |
| Solvent | 67.73 | 90.39 | 39.49 |
| Number of TLS groups | 35 | 33 | 19 |

| | SP-BC | | SP-BC | |
|---|---|---|---|---|
| | (low pH) | | (high pH) | |
| Wavelength | 0.979 | | 0.979 | |
| Resolution range | 51.96-1.9 | (1.968-1.9) | 43.33-1.755 | (1.817-1.755) |
| Space group | P 1 21 1 | | P 21 21 21 | |
| Unit cell | 52.193 | | 38.396 | |
| | 79.008 | | 58.36 | |
| | 71.917 90 | | 64.673 90 | |
| | 106.428 90 | | 90 90 | |
| Total reflections | 150936 | (14934) | 95769 | (7596) |
| Unique reflections | 43545 | (4075) | 14990 | (1393) |
| Multiplicity | 3.5 | (3.5) | 6.4 | (5.4) |
| Completeness (%) | 97.42 | (93.14) | 98.91 | (95.21) |
| Mean I/sigma(I) | 9.54 | (1.09) | 16.52 | (1.35) |
| Wilson B-factor | 36.20 | | 30.31 | |
| R-merge | 0.1057 | (1.102) | 0.09072 | (1.229) |
| R-meas | 0.1252 | (1.301) | 0.09891 | (1.357) |
| R-pim | 0.06629 | (0.687) | 0.03881 | (0.5642) |
| CC1/2 | 0.991 | (0.211) | 0.998 | (0.495) |
| CC* | 0.998 | (0.59) | 1 | (0.814) |
| Reflections | 43042 | (4071) | 14923 | (1392) |
| used in | | | | |
| refinement | | | | |
| Reflections | 2127 | (184) | 703 | (64) |
| used for | | | | |
| R-free | | | | |
| R-work | 0.2105 | (0.3532) | 0.1773 | (0.3236) |
| R-free | 0.2392 | (0.3642) | 0.2121 | (0.3531) |
| CC(work) | 0.947 | (0.580) | 0.963 | (0.788) |
| CC(free) | 0.938 | (0.517) | 0.955 | (0.681) |

| | | |
|---|---|---|
| Number of | 4862 | 1297 |
| non-hydrogen | | |
| atoms | | |
| Macromolecules | 4680 | 1196 |
| Ligands | 15 | 1 |
| Solvent | 167 | 100 |
| Protein residues | 586 | 150 |
| RMS(bonds) | 0.004 | 0.013 |
| RMS(angles) | 0.74 | 1.04 |
| Ramachandran | 97.72 | 97.95 |
| favored (%) | | |
| Ramachandran | 2.11 | 2.05 |
| allowed (%) | | |
| Ramachandran | 0.18 | 0.00 |
| outliers (%) | | |
| Rotamer outliers | 1.55 | 0.76 |
| (%) | | |
| Clashscore | 4.50 | 1.72 |
| Average B-factor | 49.21 | 43.84 |
| Macromolecules | 49.18 | 43.19 |
| Ligands | 77.91 | 33.81 |
| Solvent | 47.58 | 51.75 |
| Number of TLS groups | 30 | 10 |

Statistics for the highest-resolution shell are shown in parentheses.

TABLE 2
Phospholipid analysis of SP-BN and SP-BC purified from E. coli.

| | | | SP-BN | SP-BC |
|---|---|---|---|---|
| Lipid | Neutral Mass | Formula | (ion intensity) | (ion intensity) |
| PE(16:1/18:1) | 715.5152 | C39 H74 O8 N1 P1 | 2.36E+08 | 2.81E+05 |
| PE(18:0/18:2) | 743.5465 | C41 H78 O8 N1 P1 | 2.34E+08 | 2.14E+05 |
| PG(18:0/18:2) | 774.5411 | C42 H79 O10 N0 P1 | 1.45E+08 | 3.75E+03 |
| PE(16:0/16:1) | 689.4996 | C37 H72 O8 N1 P1 | 1.39E+08 | 1.50E+05 |
| PS(18:0/18:0) | 791.5676 | C42 H82 O10 N1 P1 | 1.06E+08 | 2.03E+04 |
| PE(23:0/10:1) | 703.5152 | C38 H74 O8 N1 P1 | 9.68E+07 | 7.43E+04 |
| PG(16:0/18:2) | 746.5098 | C40 H75 O10 N0 P1 | 5.74E+07 | 0.00E+00 |
| PE(17:0/18:2) | 729.5309 | C40 H76 O8 N1 P1 | 5.53E+07 | 1.52E+05 |
| PE(16:0/18:1) | 717.5309 | C39 H76 O8 N1 P1 | 3.51E+07 | 1.50E+04 |
| PS(18:0/16:0) | 763.5363 | C40 H78 O10 N1 P1 | 3.48E+07 | 0.00E+00 |
| PG(16:0/16:1) | 720.4941 | C38 H73 O10 N0 P1 | 3.20E+07 | 0.00E+00 |
| PE(25:1/11:4) | 737.4996 | C41 H72 O8 N1 P1 | 2.68E+07 | 0.00E+00 |
| PG(16:1/17:0) | 734.5098 | C39 H75 O10 N0 P1 | 1.20E+07 | 0.00E+00 |
| PG(17:0/18:2) | 760.5254 | C41 H77 O10 N0 P1 | 6.60E+06 | 0.00E+00 |
| PE(16:1/16:1) | 687.4839 | C37 H70 O8 N1 P1 | 5.01E+06 | 0.00E+00 |
| PE(8:0p/10:0) | 479.3012 | C23 H46 O7 N1 P1 | 2.60E+06 | 1.52E+04 |

TABLE 3
Viability of mice with mutations in the SP-BN domain
(knock-in of the L95K/L104E/V139K mutations)

| Genotype | Number of mice | Phenotype |
|---|---|---|
| INDEL | 2 | dead |
| Knock-in plus 6-bp deletion | 1 | dead |
| Knock-in/INDEL | 2 | dead |

TABLE 4
Viability of mice with mutations in the SP-BN
domain (knock-in of the K105E/R110E mutations)

| Genotype | Number of mice | Phenotype |
|---|---|---|
| WT | 8 | alive |
| INDEL | 2 | dead |
| K105E | 2 | alive |
| K105E/Knock-in | 1 | alive |
| Knock-in | 3 | alive |

SEQUENCES

SEQ ID NO: 1: SP-B (Homo sapiens) amino acid sequence
MAESHLLQWL LLLLPTLCGP GTAAWTTSSL ACAQGPEFWC QSLEQALQCR ALGHCLQEVW
GHVGADDLCQ ECEDIVHILN KMAKEAIFQD TMRKFLEQEC NVLPLKLLMP QCNQVLDDYF
PLVIDYFQNQ TDSNGICMHL GLCKSRQPEP EQEPGMSDPL PKPLRDPLPD PLLDKLVLPV
LPGALQARPG PHTQDLSEQQ FPIPLPYCWL CRALIKRIQA MIPKGALAVA VAQVCRVVPL
VAGGICQCLA ERYSVILLDT LLGRMLPQLV CRLVLRCSMD DSAGPRSPTG EWLPRDSECH
LCMSVTTQAG NSSEQAIPQA MLQACVGSWL DREKCKQFVE QHTPQLLTLV PRGWDAHTTC
QALGVCGTMS SPLQCIHSPD L
SEQ ID NO: 2: SP-BN (Homo sapiens) amino acid sequence
HVGADDLCQ ECEDIVHILN KMAKEAIFQD TMRKFLEQEC NVLPLKLLMP QCNQVLDDYF
PLVIDYFQNQ TDSNGICMHL GLCKSRQ
SEQ ID NO: 3: SP-BM (Homo sapiens) amino acid sequence
FPIPLPYCWL CRALIKRIQA MIPKGALAVA VAQVCRVVPL VAGGICQCLA ERYSVILLDT
LLGRMLPQLV CRLVLRCSM
SEQ ID NO: 4: GST-SPBN-TEV-SPBM (TEV site in bold, Thrombin site is LVPRGS) amino
acid sequence
MSPILGYWKIKGLVQPTRLLLEYLEEKYEEHLYERDEGDKWRNKKFELGLEFPNLPYYIDGDVKL
TQSMAIIRYIADKHNMLGGCPKERAEISMLEGAVLDIRYGVSRIAYSKDFETLKVDFLSKLPEML
KMFEDRLCHKTYLNGDHVTHPDFMLYDALDVVLYMDPMCLDAFPKLVCFKKRIEAIPQIDKYLKS
SKYIAWPLQGWQATFGGGDHPPKSDLEVLFQGPLGSPEFAWTTSSLACAQGPEFWCQSLEQALQC
RALGHCLQEVWGHVGADDLCQECEDIVHILNKMAKEAIFQDTMRKFLEQECNVLPLKLLMPQCNQ
VLDDYFPLVIDYFQNQTDSNGICMHLGLCKSRQPEPEQEPGMSDPLPKPLRDPLPDPLLDKLVLP
VLPGALQARPGPHTQDLSEQQFENLYFQGPIPLPYCWLCRALIKRIQAMIPKGALAVAVAQVCRV
VPLVAGGICQCLAERYSVILLDTLLGRMLPQLVCRLVLRCSM
SEQ ID NO: 5: Tobacco Etch virus (TEV) protease cleavage site amino acid sequence
ENLYFQS
SEQ ID NO: 6: SP-BC (Homo sapiens) amino acid sequence
DDSAGPRSPTG EWLPRDSECH LCMSVTTQAG NSSEQAIPQA MLQACVGSWL DREKCKQFVE
QHTPQLLTLV PRGWDAHTTC QALGVCGTMS SPLQCIHSPD L
SEQ ID NO: 7: SFTPB surfactant protein B [Homo sapiens (human)]
NCBI Gene ID: 6439- Position c85668741-85657307 on human chromosome 2
SEQ ID NO: 8: Homo sapiens surfactant protein B (SFTPB), transcript variant 1, mRNA
NCBI Reference Sequence: NM_000542.5
aggctgcaga ggtgccatgg ctgagtcaca cctgctgcag tggctgctgc tgctgctgcc
cacgctctgt ggcccaggca ctgctgcctg gaccacctca tccttggcct gtgcccaggg
ccctgagttc tggtgccaaa gcctggagca agcattgcag tgcagagccc tagggcattg
cctacaggaa gtctggggac atgtgggagc cgatgaccta tgccaagagt gtgaggacat
cgtccacatc cttaacaaga tggccaagga ggccattttc caggacacga tgaggaagtt
cctggagcag gagtgcaacg tcctcccctt gaagctgctc atgccccagt gcaaccaagt
gcttgacgac tacttccccc tggtcatcga ctacttccag aaccagactg actcaaacgg
catctgtatg cacctgggcc tgtgcaaatc ccggcagcca gagccagagc aggagccagg
gatgtcagac cccctgccca aacctctgcg ggaccctctg ccagaccctc tgctggacaa
gctcgtcctc cctgtgctgc ccggggccct ccaggcgagg cctgggcctc acacacagga
tctctccgag cagcaattcc ccattcctct cccctattgc tggctctgca gggctctgat
caagcggatc caagccatga ttcccaaggg tgcgctagct gtggcagtgg cccaggtgtg
ccgcgtggta cctctggtgg cgggcggcat ctgccagtgc ctggctgagc gctactccgt
catcctgctc gacacgctgc tgggccgcat gctgccccag ctggtctgcc gcctcgtcct
ccggtgctcc atggatgaca gcgctggccc aaggtcgccg acaggagaat ggctgccgcg
agactctgag tgccacctct gcatgtccgt gaccacccag gccgggaaca gcagcgagca
ggccatacca caggcaatgc tccaggcctg tgttggctcc tggctggaca gggaaaagtg
caagcaattt gtggagcagc acacgcccca gctgctgacc ctggtgccca ggggctggga
tgcccacacc acctgccagg ccctcggggt gtgtgggacc atgtccagcc ctctccagtg
tatccacagc cccgaccttt gatgagaact cagctgtcca gctgcaaagg aaaagccaag
tgagacgggc tctgggacca tggtgaccag gctcttcccc tgctccctgg ccctcgccag
ctgccaggct gaaaagaagc ctcagctccc acaccgccct cctcaccgcc cttcctcggc
agtcacttcc actggtggac cacgggcccc cagccctgtg tcggccttgt ctgtctcagc
tcaaccacag tctgacacca gagcccactt ccatcctctc tggtgtgagg cacagcgagg
gcagcatctg gaggagctct gcagcctcca cacctaccac gacctcccag ggctgggctc
aggaaaaacc agccactgct ttacaggaca gggggttgaa gctgagcccc gcctcacacc
cacccccatg cactcaaaga ttggatttta cagctacttg caattcaaaa ttcagaagaa
taaaaaatgg gaacatacag aactctaaaa gatagacatc agaaattgtt aagttaagct
ttttcaaaaa atcagcaatt ccccagcgta gtcaagggtg gacactgcac gctctggcat
gatgggatgg cgaccgggca agctttcttc ctcgagatgc tctgctgctt gagagctatt
gctttgttaa gatataaaaa ggggtttctt tttgtctttc tgtaaggtgg acttccagct
tttgattgaa agtcctaggg tgattctatt tctgctgtga tttatctgct gaaagctcag
ctggggttgt gcaagctagg gacccattcc tgtgtaatac aatgtctgca ccaatgctaa
taaagtccta ttctctttta tgagaaagaa aaagacaccg tcctttaaag tgctgcagta
tggccagacg tggtggctca cacctgcaat cccagcacct taggaggccg aggcaggagg
atccttgagg tcaggagttc gagaccagcc tcgccaacat ggtgaaaccc catttctact
aaaaatacaa aaaattagcc aagtgtggtg gcatatgcct gtaatcccaa ctactcagaa
ggccgaggca ggagaattac ttgaacgcag gagaatcact gcagcccagg aggcagaggt
tgcagtgagc cgagattgca ccactgcact ccagcctggg tgacagagca agactccatc
tcagtaaata aataaataaa taaaaagcgc tgcagtagct gtggcctcac cctgaagtca
gcgggcccag gcctacctca ctctctccct tggcagagaa gcagacgtcc atagctcctc
tccctcacaa gcgctcccag cctgccctcc agctgctgct ctcccctccc agtctctact
cactgggatg aggttaggtc atgaggacac caaaaaccta aaaataaaca aaaagccaaa
caagccttag cttttcttaa agactgaaat gcctggaagt gtccctttat ttataaaata
acttttgtca tatttcttat acatgtttct tgtaagaaat tcagaaacta cagacaaaga
gagtggaaat tacccactgt caggcctctg agcccaagct aagccatcat atcccctgtg
ccctgcacgt atacacccag atggcctgaa gcaactgaag atccacaaaa gaagtgaaaa
tagccagttc ctgccttaac tgatgacatt ccaccattgt gatttgttcc tgccccaccc
taactgatca attgaccttg tgacaataca ccttccccac ccttgagaag gtgctttgta
atattctccc cacccacccc acgcccgcac ccccgcaccc ttaagaaggt attttgtaat
attctctccg ccattgagaa tgtgctttgt aagatccacc ccctgcccac aaaaaattgc
tcctaactcc accgcctatc ccaaacctac aagaactaat gataatccca ccaccctttg
ctgactcttt ttggactcag cccacctgca cccaggtgat taaaaagctt tattgttcac
acaaagcctg tttggtagtc tcttcacagg gaagcatgtg acacccacaa tcccacctag
cccaggagag agctacggca gggtgtgtgt tttgacactg agcttggggc tttttccatc
ttctccccac agcctctggc tccacacctc caccgttcaa gcgccagaaa gagctgtcta
tgcagcctgc tcttgggcct ggggatgaga cacacaattc attggctcct ggattttaag
tagacatttg taaatctata gctaactact gtccttaaag ccattgtttc cattacaaaa
tccaactctc tgagagaaaa gggtgtttta aatttaaaaa aataaaaaca aaaaagtttg
attgagacaa
SEQ ID NO: 9: Homo sapiens surfactant protein B (SFTPB), transcript variant 2, mRNA
NCBI Reference Sequence: NM_198843.3
tgtaaatgct cttctgacta atgcaaacca tgtgtccata gaaccagaag atttttccag
gggaaaagag cccccacgcc ccgcccagct ataaggggcc atgcaccaag cagggtaccc
aggctgcaga ggtgccatgg ctgagtcaca cctgctgcag tggctgctgc tgctgctgcc
cacgctctgt ggcccaggca ctgctgcctg gaccacctca tccttggcct gtgcccaggg
ccctgagttc tggtgccaaa gcctggagca agcattgcag tgcagagccc tagggcattg
cctacaggaa gtctggggac atgtgggagc cgatgaccta tgccaagagt gtgaggacat
cgtccacatc cttaacaaga tggccaagga ggccattttc caggacacga tgaggaagtt
cctggagcag gagtgcaacg tcctcccctt gaagctgctc atgccccagt gcaaccaagt
gcttgacgac tacttccccc tggtcatcga ctacttccag aaccagactg actcaaacgg
catctgtatg cacctgggcc tgtgcaaatc ccggcagcca gagccagagc aggagccagg
gatgtcagac cccctgccca aacctctgcg ggaccctctg ccagaccctc tgctggacaa
gctcgtcctc cctgtgctgc ccggggccct ccaggcgagg cctgggcctc acacacagga
tctctccgag cagcaattcc ccattcctct cccctattgc tggctctgca gggctctgat
caagcggatc caagccatga ttcccaaggg tgcgctagct gtggcagtgg cccaggtgtg
ccgcgtggta cctctggtgg cgggcggcat ctgccagtgc ctggctgagc gctactccgt
catcctgctc gacacgctgc tgggccgcat gctgccccag ctggtctgcc gcctcgtcct
ccggtgctcc atggatgaca gcgctggccc aaggtcgccg acaggagaat ggctgccgcg
agactctgag tgccacctct gcatgtccgt gaccacccag gccgggaaca gcagcgagca
ggccatacca caggcaatgc tccaggcctg tgttggctcc tggctggaca gggaaaagtg
caagcaattt gtggagcagc acacgcccca gctgctgacc ctggtgccca ggggctggga
tgcccacacc acctgccagg ccctcggggt gtgtgggacc atgtccagcc ctctccagtg
tatccacagc cccgaccttt gatgagaact cagctgtcca gaaaaagaca ccgtccttta
aagtgctgca gtatggccag acgtggtggc tcacacctgc aatcccagca ccttaggagg
ccgaggcagg aggatccttg aggtcaggag ttcgagacca gcctcgccaa catggtgaaa
ccccatttct actaaaaata caaaaaatta gccaagtgtg gtggcatatg cctgtaatcc
caactactca gaaggccgag gcaggagaat tacttgaacg caggagaatc actgcagccc
aggaggcaga ggttgcagtg agccgagatt gcaccactgc actccagcct gggtgacaga
gcaagactcc atctcagtaa ataaataaat aaataaaaag cgctgcagta gctgtggcct
caccctgaag tcagcgggcc caggcctacc tcactctctc ccttggcaga gaagcagacg
tccatagctc ctctccctca caagcgctcc cagcctgccc tccagctgct gctctcccct
cccagtctct actcactggg atgaggttag gtcatgagga caccaaaaac ctaaaaataa
acaaaaagcc aaacaagcct tagcttttct taaagactga aatgcctgga agtgtccctt
tatttataaa ataacttttg tcatatttct tatacatgtt tcttgtaaga aattcagaaa
ctacagacaa agagagtgga aattacccac tgtcaggcct ctgagcccaa gctaagccat
catatcccct gtgccctgca cgtatacacc cagatggcct gaagcaactg aagatccaca
aaagaagtga aaatagccag ttcctgcctt aactgatgac attccaccat tgtgatttgt
tcctgcccca ccctaactga tcaattgacc ttgtgacaat acaccttccc cacccttgag
aaggtgcttt gtaatattct ccccacccac cccacgcccg cacccccgca cccttaagaa
ggtattttgt aatattctct ccgccattga gaatgtgctt tgtaagatcc accccctgcc
cacaaaaaat tgctcctaac tccaccgcct atcccaaacc tacaagaact aatgataatc
ccaccaccct ttgctgactc tttttggact cagcccacct gcacccaggt gattaaaaag
ctttattgtt cacacaaagc ctgtttggta gtctcttcac agggaagcat gtgacaccca
caatcccacc tagcccagga gagagctacg gcagggtgtg tgttttgaca ctgagcttgg
ggctttttcc atcttctccc cacagcctct ggctccacac ctccaccgtt caagcgccag
aaagagctgt ctatgcagcc tgctcttggg cctggggatg agacacacaa ttcattggct
cctggatttt aagtagacat ttgtaaatct atagctaact actgtcctta aagccattgt
ttccattaca aaatccaact ctctgagaga aaagggtgtt ttaaatttaa aaaaataaaa
acaaaaaagt ttgattgaga caatgaagag
SEQ ID NO: 10: Homo sapiens surfactant protein B (SFTPB), transcript variant 3, mRNA
NCBI Reference Sequence: NM_001367281.1
aggctgcaga ggtgccatgg ctgagtcaca cctgctgcag tggctgctgc tgctgctgcc
cacgctctgt ggcccaggca ctgctgcctg gaccacctca tccttggcct gtgcccaggg
ccctgagttc tggtgccaaa gcctggagca agcattgcag tgcagagccc tagggcattg
cctacaggaa gtctggggac atgtgggagc cgatgaccta tgccaagagt gtgaggacat
cgtccacatc cttaacaaga tggccaagga ggccattttc caggacacga tgaggaagtt
cctggagcag gagtgcaacg tcctcccctt gaagctgctc atgccccagt gcaaccaagt
gcttgacgac tacttccccc tggtcatcga ctacttccag aaccagactg actcaaacgg
catctgtatg cacctgggcc tgtgcaaatc ccggcagcca gagccagagc aggagccagg
gatgtcagac cccctgccca aacctctgcg ggaccctctg ccagaccctc tgctggacaa
gctcgtcctc cctgtgctgc ccggggccct ccaggcgagg cctgggcctc acacacagga
tctctccgag cagcaattcc ccattcctct cccctattgc tggctctgca gggctctgat
caagcggatc caagccatga ttcccaaggg tgcgctagct gtggcagtgg cccaggtgtg
ccgcgtggta cctctggtgg cgggcggcat ctgccagtgc ctggctgagc gctactccgt
catcctgctc gacacgctgc tgggccgcat gctgccccag ctggtctgcc gcctcgtcct
ccggtgctcc atggatgaca gcgctggccc aaggtcgccg acaggagaat ggctgccgcg
agactctgag tgccacctct gcatgtccgt gaccacccag gccgggaaca gcagcgagca
ggccatacca caggcaatgc tccaggcctg tgttggctcc tggctggaca gggaaaagaa
aaagacaccg tcctttaaag tgctgcagta tggccagacg tggtggctca cacctgcaat
cccagcacct taggaggccg aggcaggagg atccttgagg tcaggagttc gagaccagcc
tcgccaacat ggtgaaaccc catttctact aaaaatacaa aaaattagcc aagtgtggtg
gcatatgcct gtaatcccaa ctactcagaa ggccgaggca ggagaattac ttgaacgcag
gagaatcact gcagcccagg aggcagaggt tgcagtgagc cgagattgca ccactgcact
ccagcctggg tgacagagca agactccatc tcagtaaata aataaataaa taaaaagcgc
tgcagtagct gtggcctcac cctgaagtca gcgggcccag gcctacctca ctctctccct
tggcagagaa gcagacgtcc atagctcctc tccctcacaa gcgctcccag cctgccctcc
agctgctgct ctcccctccc agtctctact cactgggatg aggttaggtc atgaggacac
caaaaaccta aaaataaaca aaaagccaaa caagccttag cttttcttaa agactgaaat
gcctggaagt gtccctttat ttataaaata acttttgtca tatttcttat acatgtttct
tgtaagaaat tcagaaacta cagacaaaga gagtggaaat tacccactgt caggcctctg
agcccaagct aagccatcat atcccctgtg ccctgcacgt atacacccag atggcctgaa
gcaactgaag atccacaaaa gaagtgaaaa tagccagttc ctgccttaac tgatgacatt
ccaccattgt gatttgttcc tgccccaccc taactgatca attgaccttg tgacaataca
ccttccccac ccttgagaag gtgctttgta atattctccc cacccacccc acgcccgcac
ccccgcaccc ttaagaaggt attttgtaat attctctccg ccattgagaa tgtgctttgt
aagatccacc ccctgcccac aaaaaattgc tcctaactcc accgcctatc ccaaacctac
aagaactaat gataatccca ccaccctttg ctgactcttt ttggactcag cccacctgca
cccaggtgat taaaaagctt tattgttcac acaaagcctg tttggtagtc tcttcacagg
gaagcatgtg acacccacaa tcccacctag cccaggagag agctacggca gggtgtgtgt
tttgacactg agcttggggc tttttccatc ttctccccac agcctctggc tccacacctc
caccgttcaa gcgccagaaa gagctgtcta tgcagcctgc tcttgggcct ggggatgaga
cacacaattc attggctcct ggattttaag tagacatttg taaatctata gctaactact
gtccttaaag ccattgtttc cattacaaaa tccaactctc tgagagaaaa gggtgtttta
aatttaaaaa aataaaaaca aaaaagtttg attgagacaa tgaagag
SEQ ID NO: 11: pulmonary surfactant-associated protein B isoform 2 precursor [Homo
sapiens]
NCBI Reference Sequence: NP_001354210.1
MAESHLLQWL LLLLPTLCGP GTAAWTTSSL ACAQGPEFWC QSLEQALQCR ALGHCLQEVW
GHVGADDLCQ ECEDIVHILN KMAKEAIFQD TMRKFLEQEC NVLPLKLLMP QCNQVLDDYF
PLVIDYFQNQ TDSNGICMHL GLCKSRQPEP EQEPGMSDPL PKPLRDPLPD PLLDKLVLPV
LPGALQARPG PHTQDLSEQQ FPIPLPYCWL CRALIKRIQA MIPKGALAVA VAQVCRVVPL
VAGGICQCLA ERYSVILLDT LLGRMLPQLV CRLVLRCSMD DSAGPRSPTG EWLPRDSECH
LCMSVTTQAG NSSEQAIPQA MLQACVGSWL DREKKKTPSF KVLQYGQTWW LTPAIPAP
SEQ ID NO: 12: pulmonary surfactant-associated protein B isoform 1 precursor [Homo
sapiens]
NCBI Reference Sequence: NP_942140.3
MAESHLLQWL LLLLPTLCGP GTAAWTTSSL ACAQGPEFWC QSLEQALQCR ALGHCLQEVW
GHVGADDLCQ ECEDIVHILN KMAKEAIFQD TMRKFLEQEC NVLPLKLLMP QCNQVLDDYF
PLVIDYFQNQ TDSNGICMHL GLCKSRQPEP EQEPGMSDPL PKPLRDPLPD PLLDKLVLPV
LPGALQARPG PHTQDLSEQQ FPIPLPYCWL CRALIKRIQA MIPKGALAVA VAQVCRVVPL
VAGGICQCLA ERYSVILLDT LLGRMLPQLV CRLVLRCSMD DSAGPRSPTG EWLPRDSECH
LCMSVTTQAG NSSEQAIPQA MLQACVGSWL DREKCKQFVE QHTPQLLTLV PRGWDAHTTC
QALGVCGTMS SPLQCIHSPD L
SEQ ID NO: 13: pulmonary surfactant-associated protein C isoform 1 precursor[Homo
sapiens]
NCBI Reference Sequence: NP_003009.2
MDVGSKEVLM ESPPDYSAAP RGRFGIPCCP VHLKRLLIVV VVVVLIVVVI VGALLMGLHM
SQKHTEMVLE MSIGAPEAQQ RLALSEHLVT TATFSIGSTG LVVYDYQQLL IAYKPAPGTC
CYIMKIAPES IPSLEALTRK VHNFQMECSL QAKPAVPTSK LGQAEGRDAG SAPSGGDPAF
LGMAVSTLCG EVPLYYI
SEQ ID NO: 14: pulmonary surfactant-associated protein C isoform 2 [Homo sapiens]
NCBI Reference Sequence: NP_001304707.1
MDVGSKEVLM ESPPDYSAAP RGRFGIPCCP VHLKRLLIVV VVVVLIVVVI VGALLMGLHM
SQKHTEMVLE MSIGAPEAQQ RLALSEHLVT TATFSIGSTG LVVYDYQQLL IAYKPAPGTC
CYIMKIAPES IPSLEALTRK VHNFQAKPAV PTSKLGQAEG RDAGSAPSGG DPAFLGMAVS
TLCGEVPLYY I
SEQ ID NO: 15: proactivator polypeptide-like 1 preproprotein [Homo sapiens]
NCBI Reference Sequence: NP_001078851.1
MLCALLLLPS LLGATRASPT SGPQECAKGS TVWCQDLQTA ARCGAVGYCQ GAVWNKPTAK
SLPCDVCQDI AAAAGNGLNP DATESDILAL VMKTCEWLPS QESSAGCKWM VDAHSSAILS
MLRGAPDSAP AQVCTALSLC EPLQRHLATL RPLSKEDTFE AVAPFMANGP LTFHPRQAPE
GALCQDCVRQ VSRLQEAVRS NLTLADLNIQ EQCESLGPGL AVLCKNYLFQ FFVPADQALR
LLPPQELCRK GGFCEELGAP ARLTQVVAMD GVPSLELGLP RKQSEMQMKA GVTCEVCMNV
VQKLDHWLMS NSSELMITHA LERVCSVMPA SITKECIILV DTYSPSLVQL VAKITPEKVC
KFIRLCGNRR RARAVHDAYA IVPSPEWDAE NQGSFCNGCK RLLTVSSHNL ESKSTKRDIL
VAFKGGCSIL PLPYMIQCKH FVTQYEPVLI ESLKDMMDPV AVCKKVGACH GPRTPLLGTD
QCALGPSFWC RSQEAAKLCN AVQHCQKHVW KEMHLHAGEH A
SEQ ID NO: 16: N- terminal His₆ tagged-GST-SPBN-TEV-SPBM (TEV site in bold,
Thrombin site is LVPRGS) amino acid sequence:
MHHHHHHAGMSPILGYWKIKGLVQPTRLLLEYLEEKYEEHLYERDEGDKWRNKKFELGLEFPNLP
YYIDGDVKLTQSMAIIRYIADKHNMLGGCPKERAEISMLEGAVLDIRYGVSRIAYSKDFETLKVD
FLSKLPEMLKMFEDRLCHKTYLNGDHVTHPDFMLYDALDVVLYMDPMCLDAFPKLVCFKKRIEAI
PQIDKYLKSSKYIAWPLQGWQATFGGGDHPPKSDLEVLFQGPLGSPEFAWTTSSLACAQGPEFWC
QSLEQALQCRALGHCLQEVWGHVGADDLCQECEDIVHILNKMAKEAIFQDTMRKFLEQECNVLPL
KLLMPQCNQVLDDYFPLVIDYFNQTDSNGICMHLGLCKSRQPEPEQEPGMSDPLPKPLRDPLPDP
LLDKLVLPVLPGALQARPGPHTQDLSEQQFLVPRGSPIPLPYCWLCRALIKRIQAMIPKGALAVA
VAQVCRVVPLVAGGICQCLAERYSVILLDTLLGRMLPQLVCRLVLRCSM

What is claimed is:

1. A lung surfactant composition comprising:
a. a polypeptide comprising a surfactant B protein N-terminal domain (SP-BN); and
b. at least one phospholipid.

2. The lung surfactant composition of claim 1, wherein the polypeptide further comprises a surfactant B protein middle domain (SP-BM) and/or surfactant B protein C-terminal domain (SP-BC).

3. A lung surfactant composition comprising:
a. a polypeptide comprising a surfactant B protein middle domain (SP-BM); and
b. at least one phospholipid

4. The lung surfactant composition of claim 3, wherein the polypeptide lacks a surfactant B protein N-terminal domain (SP-BN) and/or a surfactant B protein C-terminal domain (SP-BC).

5. The lung surfactant composition of claim 1, wherein the phospholipid is a glycerophospholipid.

6. The lung surfactant composition of claim 1, wherein the phospholipid is selected from the group consisting of 1,2-dipalmitoyl-sn-glycero-3-phosphocholine (DPPC); phosphatidylglycerol (PG); phosphatidylcholine (PC), phosphatidylethanolamine (PE), phosphatidylserine (PS), and phosphatidylinositol (PI).

7. The lung surfactant composition of claim 1, wherein the phospholipid is DPPC, PG, or a combination thereof.

8. The lung surfactant composition of claim 1, wherein the SP-BN is human, bovine, or mouse SP-BN.

9. The lung surfactant composition of claim 2, wherein the SP-BM is human, bovine, or mouse SP-BM.

10. The lung surfactant composition of claim 1, wherein the composition is formulated with a pharmaceutically acceptable carrier.

11. The lung surfactant composition of claim 1, wherein the composition is formulated for intratracheal delivery.

12. The lung surfactant composition of claim 1, further comprising a Surfactant Protein C (SP-C) or a fragment thereof; and/or an additional lung surfactant.

13. The lung surfactant composition of claim 1, wherein the SP-BN, the SP-BM, or the SP-BC comprises one or more amino acid substitutions or mutations.

14. The lung surfactant composition of claim 13, wherein the SP-BM comprises a C₄₈Δ amino acid substitution.

15. The lung surfactant composition of claim 13, wherein the SP-BN comprises a K46E, P50C, R51E, Y59A, and/or a H79Δ amino acid substitution, or any combination thereof.

16. A fusion protein comprising:
i. a peptide tag;
ii. a surfactant B protein N-terminal domain (SP-BN);
iii. a linker; and
iv. a surfactant B protein middle domain (SP-BM) domain.

17. The fusion protein of claim 16, where the linker comprises a cleavage site.

18. The fusion protein of claim 16, wherein cleavage site is a protease cleavage site.

19. The fusion protein of claim 16, wherein the fusion protein lacks a surfactant B protein C-terminal domain (SP-BC).

20. The fusion protein of claim 16, wherein the fusion protein further comprises a saposin polypeptide between the peptide tag and the SP-BN domain.


SEQUENCE LISTING (27 sequences)

SEQ ID NO: 1 - protein, 381 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 2 - protein, 86 aa, Homo sapiens
HVGADDLCQECEDIVHILNKMAKEAIFQDTMRKFLEQECNVLPLKLLMPQCNQVLDDYFPLVIDYFQNQTDSNGICMHLGLCKSRQ

SEQ ID NO: 3 - protein, 79 aa, Homo sapiens
FPIPLPYCWLCRALIKRIQAMIPKGALAVAVAQVCRVVPLVAGGICQCLAERYSVILLDTLLGRMLPQLVCRLVLRCSM

SEQ ID NO: 4 - protein, 497 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPEFAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFENLYFQGPIPLPYCWLCRALIKRIQAMIPKGALAVAVAQVCRVVPLVAGGICQCLAERYSVILLDTLLGRMLPQLVCRLVLRCSM

SEQ ID NO: 5 - protein, 7 aa, Tobacco etch virus
ENLYFQS

SEQ ID NO: 6 - protein, 102 aa, Homo sapiens
DDSAGPRSPTGEWLPRDSECHLCMSVTTQAGNSSEQAIPQAMLQACVGSWLDREKCKQFVEQHTPQLLTLVPRGWDAHTTCQALGVCGTMSSPLQCIHSPDL

SEQ ID NO: 7 - DNA, 11435 bp, Homo sapiens
tgtaaatgctcttctgactaatgcaaaccatgtgtccatagaaccagaagatttttccaggggaaaaggtaaggaggtggtgagagtgtcctgggtctgcccttccagggcttgccctgggttaagagccaggcaggaagctctcaagagcattgctcaagagtagagggggcctgggaggcccagggaggggatgggaggggaacacccaggctgcccccaaccagatgccctccaccctcctcaacctccctcccacggcctggagaggtgggaccaggtatggaggcttgagagcccctggttggaggaagccacaagtccaggaacatgggagtctgggcagggggcaaaggaggcaggaacaggccatcagccaggacaggtggtaaggcaggcaggagtgttcctgctgggaaaaggtgggatcaagcacctggagggctcttcagagcaaagacaaacactgaggtcgctgccactcctacagagcccccacgccccgcccagctataaggggccatgcaccaagcagggtacccaggctgcagaggtgccatggctgagtcacacctgctgcagtggctgctgctgctgctgcccacgctctgtggcccaggcactggtgagtctcccccagcctcccctctcctaggcagctccaccactcactgagcactgctttgtgctaggcattaacccaagtctgtcctcattttaaagacaaggcagctggggttcagagagggttcagagcttatccaaggtcacacagctggcgggtccaggagcaggtggaacccagagctgtctgacgtccacatgtttaatggcctcacactcccagcaaaactgggtctagagggtgggtgaaatcatgatgccaggtgtgtagcctggatcctgattaaggttgctctggccccaaaccacagctgcctggaccacctcatccttggcctgtgcccagggccctgagttctggtgccaaagcctggagcaagcattgcagtgcagagccctagggcattgcctacaggaagtctggggacatgtgggagccgtgagtaccaccaaggatgcatggcaactgggggtctgaaatgaagggtgctgggtgggctctggatgggcaggaggagagtggagcccccataggggatggatgagatgaaatgggatgagatgaaatgagataggataaaatggaatgggatggatgcgatgggatacgatgacatagaatagatggagtcggatgaatgggatgggatgggatggatgggaggggaagggataggataggatgacatagaataaagatggatgggatgggatgggatgggatgggatgacacagaataaagatggatggattgggatggatgaatagaagagatggatgggataaattgatatggatgagatgggacaagttgggctggtgggcagctgcatgtgccttggagtgctctgttggcctcttcctaagagaacctccccattggagctgggagcctcccccactcatgtgtcctccaccttggggcccctccctccccaggatgacctatgccaagagtgtgaggacatcgtccacatccttaacaagatggccaaggaggccattttccaggtaatgatgcccagatcctggatgaaggttggggcccaagagatgagggacagagcagggaagagctgagccccctaaaggggccatttccaggctgaggaggaggcctgggtgcctgggaagtcccagctcctcctggctgggagcaggtcatggccctgagctcaatagcacagccagagatggtcttccctgaggggaagggcccctacatgtgcccaactacttaactccttggcactcgtgaactccagcaccctgggggattaggggtcagtctgccctggtggggccttgtgtccagggacttgggcggggtagacctcagagaggcccagctgacggccccctctggcctcccaggacacgatgaggaagttcctggagcaggagtgcaacgtcctccccttgaagctgctcatgccccagtgcaaccaagtgcttgacgactacttccccctggtcatcgactacttccagaaccagactgtgagggctgcaagctcacctcctgcctgcctccccacgcaggcccctgtgcccacccatggggagccacacacacagcaccccagccagccagacacacacacacacacacacacacacacagcacccaagccggccagacacaaacacacagcaccccagccagccggacacacacacacacacacacacaacaccccagctggccggacacacacacacacagtaccccagctggccggacacacacacacacagcaccctatccagacacatacacacacacagtaccccagccagctggaaacacacacacacacagcactccatccagacacatacccacacagtaccccagccagccagacacacacacacacacacacacacacacacacacagcacacacacagcaccccagctggccacacacacacacacacacaccctgtccacaaagggcctaggaaactacgtgcccttcagccatgcacccgaccatgggcccccaggttcaggtgcacacggtgggcctgtacgctcacacacccttacaccctcactctcacacacatgcttacacacttattcattctcacatatatgctcatgctcattcacacacaatcccggccacctgccctaaagtccccacacagccctatctttgccttttgtccccccacatagagttctaaaccacagcacccccactaggcctgcttcctcccattccagtggtccctgagcccttgggccggcctgaataggggtgggcttccctcccagaccctaacactcccaccctgtgctgtgccccaggactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggtgagggaggcccccacagccagtaaagtggagatccagagggctagagccacctccgaagcccatgggcactgggccctgggagaggcagagccgggaaggtgataggaagctccaggcagggcctaagggaggagggagagaaagggaggaagagagaggggaggagagcctggaggactcttctcccagcacccagcctggcctccacctgattctttccccaggatctctccgagcagcaattccccattcctctcccctattgctggctctgcagggctctgatcaagcggatccaagccatgattcccaaggtgaggcatccagggcctcacagagcccaggagcacacgcatacctgtagctccctgcagctcccacctctctcccaactcacacccccgtcaggacccagctggctgccagaagttaggaggggagagagccgcttgtgcattgcccccacccaggggaccctggggctcaggctcaggcctggtaggtgccaggcctacagttcatgcaacaaacattaagcccccactgtatggaggtgccacgccaggagccaaagtacaaaaacggacaagacgcagctttgtcctccagcagctcaccatctgatggagaaagatccccagaggtctctgtagaaaggttgctttgatctttcaagaggggaatttccacagatagattccccatccttgcctgagtccaacttggagtcttccagacctgcagtggctattgtccaatggccccgccagcccagggctaccttgcccaaattggggcccaaatgaggaaaggccctgccccctcagcctttcccagatagggttgcgtgggccaccaggggcacaaggcagcaggtgaggttcctgctgaggcaggtggttcacttgagcccaggagttcaagaccagcttgggcaacatggcgaaaccccgtctctactaagaatacaaaaattagccagatgtgacaggtgcctgtagtcccagctactcgggaggctgaggcaggagaatcacttgaacccaggaggcggaggttgcagtgagccgacatcacgccactgtactctagcctgggtgacagagcaagactctgtctcaaaaaaaaagaaagaaggaaagatcactgcagagattgcagtgagaggtgatgggacagggacggagctgagggctggcctggggatgcatttgggaggtgggcccactgctattgggcatggatgggcctggagcgtgaggaccagggaggactccaaagtgacttttacacactggccagagcaaccagccctctgtaatgccagcagctgagatggggagactaaagaagaaaacaggtttgagcaaaaaaacagagagctccctcctggccatgttgagttcaagatgcctgtgtgaagtgcaggagaggagagtcaggcaagcagctgaatcccaagcattgggggaaggtcaggtccaccatgtcagtctgagagtcactagctgtgggccagagcctttggggccagacgtaggtctgaagctggctcctacactcagtgaccctgtgtgagtcccctgcatcccctggactctctgatccccagtgtccttatttgtgaatagccttgccctcccttctagaagagaatgagggaatgcgtaggaagtgcccagctgggtgctgggcagagagtggaggcttgccaagtgaaggtcccatgctggcctctctccgcccccgccccagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaagtgagcccactgcccactccttagcccaatgcctgctctcctcctccccctaccctgccactgcatgaccctctccctctgtggtcccactgcaatgcaccaaggaggacagaaaccaaacacctctgtagggtggccttgcctgctttccccctaatgctcacatctccagggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaaggtatgggctgggcacatggggactcatggtcagggcccgttcaaggcagaaggctgagcccaggaaaggctttgcagccagagacacctaggatgggccagaatggagcacagacaggcagacaggatgtggggcagacaatggtgggactgtaagttagggcagagcctgctaaaggttaggagtcgcctctggacaaagggctgtgggctccagaggaccagcaggccctcttcacgggctgagtgagcaccaggcaagccttcagaggcctggttatctaccaggagatgagtaatgctagggccagttcaagccaggaaagggactagccttctctccagggtcctgatccctttactgcccccacactcctcaaggtgtgactcactcaggacaaacccattggcaaaaggagagggctggacttgaaggtcctagggcccttgccaatactcagtcaatgacaggaaattcccttttttttttttttttttttttttttgagatggagttttgctcttgttgcccaggctggagtgcaatggcacaatcttggctcactgcaacctctgcctccgggttcaggcgattctcctgcctcagcctcttgagtagctgggattacaggcatgtgctaccaggcccggctaatttttgtatttttagtagagacaaggtttcaccatattggtcaggctggtctcgaacccctgacctgaagtgatctgcccgccttggcctcccaaagtgctgggattacaggcataagccactgcacccggacaggaaattcccttcttaaagcgagatcctgtcctgaggaaagccagctgatgctcttcccaggaggcagctgtccacactgtgctccctgctcagcaactcccaagcctcccgactgcccatcacatctggtctcaaggaccagatgaacgttaagtttccttctagaactgaaatggaggtggagggaggggagggtggtggctgagattccacccctctgcctgagtcctccgtctccagtgtcgcctgcttttctgatggaagtcctccatttcagctggctccagtttgttaagggtttcaactgcagccagaggtgttccgtgagggctgatggaggagtcgggagggagccctagagtgatccagagatgtggagaggccaggaccacacgacaggagagtcctgcaaagggaccctccacagctgtgtgtctccttcctcagtgcaagcaatttgtggagcagcacacgccccagctgctgaccctggtgcccaggggctgggatgcccacaccacctgccaggtacacccacccctcccagttggtcctaggacttcccttggctcccagagcccccaccctttgggcccgtgatcctcagaggcctcactcccctgggtccaaggtggtcccaggtgcacgggccagggactgggaggcacccctctctgtttcagtgtaaaaaatcatgagagcatggaaaagggggatgggaagggagggatggcctgaggagtgcggctggatgtccattataggatggggctgtgttccctggccagtgtgtgctggtggggtgggggtacaaagtgggtgttctggagtgaacatctcacctcctcaggctctaaaccctaaggcctgtggctcagggagtggcccgaggggtctacagagtcacactggtagcacccactaggcgggaggtggagtgagtgctgttctttcccggaagagctgggtgtggggagctgagggggcccaggcctcagccctggtgctgtccctgtgacaggccctcggggtgtgtgggaccatgtccagccctctccagtgtatccacagccccgacctttgatgagaactcagctgtccaggtgagtccaggcccccagttgcggggaggtaagggggcaggtcctgaccatcagggcatgggaggcccttctgctccccaagcaggaagaggcggccactcctgccggctgctccatcctccctctcaccgcacagctggaggctcctgagggcttctggctggccatcaggaaaacaccctttccggaccccgagcactgccccgcccagaaccccagtcactgagtgcccaacccccagcttcccccccaaccccccgccctgccctgtcccaggcctccctctcagagcttgccccagggactctctggccctcagggttcaatgtattctgaccaaggccaagctttcctggggctcagggaaaatcacactttgctacccgaagctgtatcccctcagatgccaggaaggccgtgatcatctgactccaccctcctgagacacattctctccctgactgtcctgttctaagtcagcggagcaccttaggatggaggggtggaggcgaggccagatgcagcctctgtgaacaggtgcctggaggctgggaaatgaccctgagagggcaggacacagcaaccgtgggcttaaggtgaccttgagagcaagcttggcccactttacaattctgttcagagccagcccctaacatggtggtcatttattcatttgttccctcattttaaaaaatgtaaggccaggcatggtggctcacgccggtaatcccagcactttgggaggccgaggcaggcagatcacctgaggtcaggagttcgagactagcctggccaacatggcgaaaccctgtctctactaaaaatattttttaaaaattagctgagcatggtggcaggtgcctgtaatcccagctactcaggacgcttaggcaggagaatcacttgaacctgggaggcgaaggttgcggtgtgccgagatcgtgccactgcactctagcctaggcaacagagcacaactctgtctcaggaaaaaaaaaaaaaaaaaaaaggtatttctttgctgggcgcagtggctcacacctgtaatcccagcactttgggagaccgaggcgagtggatcacttgaggtcaggagttcaagaccagccttaccaacatgatgaaaccccgtatctactaaaaaaaaaaaaaaaaaaaaaaaaaattagccagatgtggtggcacacacctgtaatcccagctacttgggaggctgaggaggagaattgcttgaacctgggaggcggagattgcagcgagccaagattgcgcctctgcactccagcctgggtgacagagtgagactccgtctcaaaaaaaaaaaaaaaaaagtagtgggtgcctgtggccaggccacatcctagggtaggggctatggctgagccctgccctcctggagctcacagccaagtccacttcttccatctgaggcggggaagccagccctgttcctgaaaccctgcatcacaagcccctgtgggaggcagtggggaggggaggtcctcccccactcagacctgacccacagggaccagtttaatgtgtccttgccccagtgatgacagctggggatctgggggtggggagtcacccaggacccgggcagtcgcctttccccagctcctagggctcccggccttccctgctgaaacagcaagaccagtgggttggcgtgggaggcctgggcttcaaaccacctctgctatcacctggctgtgggtccccaggcaggacatacacacagtccctctctggccctcatcctcctcagctgcaaaggaaaagccaagtgagacgggctctgggaccatggtgaccaggctcttcccctgctccctggccctcgccagctgccaggctgaaaagaagcctcagctcccacaccgccctcctcaccgcccttcctcggcagtcacttccactggtggaccacgggcccccagccctgtgtcggccttgtctgtctcagctcaaccacagtctgacaccagagcccacttccatcctctctggtgtgaggcacagcgagggcagcatctggaggagctctgcagcctccacacctaccacgacctcccagggctgggctcaggaaaaaccagccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagcttttgattgaaagtcctagggtgattctatttctgctgtgatttatctgctgaaagctcagctggggttgtgcaagctagggacccattcctgtgtaatacaatgtctgcaccaatgctaataaagtcctattctcttttatgagaaagaaaaagacaccgtcctttaaagtgctgcagtatggccagacgtggtggctcacacctgcaatcccagcaccttaggaggccgaggcaggaggatccttgaggtcaggagttcgagaccagcctcgccaacatggtgaaaccccatttctactaaaaatacaaaaaattagccaagtgtggtggcatatgcctgtaatcccaactactcagaaggccgaggcaggagaattacttgaacgcaggagaatcactgcagcccaggaggcagaggttgcagtgagccgagattgcaccactgcactccagcctgggtgacagagcaagactccatctcagtaaataaataaataaataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccactgtcaggcctctgagcccaagctaagccatcatatcccctgtgccctgcacgtatacacccagatggcctgaagcaactgaagatccacaaaagaagtgaaaatagccagttcctgccttaactgatgacattccaccattgtgatttgttcctgccccaccctaactgatcaattgaccttgtgacaatacaccttccccacccttgagaaggtgctttgtaatattctccccacccaccccacgcccgcacccccgcacccttaagaaggtattttgtaatattctctccgccattgagaatgtgctttgtaagatccaccccctgcccacaaaaaattgctcctaactccaccgcctatcccaaacctacaagaactaatgataatcccaccaccctttgctgactctttttggactcagcccacctgcacccaggtgattaaaaagctttattgttcacacaaagcctgtttggtagtctcttcacagggaagcatgtgacacccacaatcccacctagcccaggagagagctacggcagggtgtgtgttttgacactgagcttggggctttttccatcttctccccacagcctctggctccacacctccaccgttcaagcgccagaaagagctgtctatgcagcctgctcttgggcctggggatgagacacacaattcattggctcctggattttaagtagacatttgtaaatctatagctaactactgtccttaaagccattgtttccattacaaaatccaactctctgagagaaaagggtgttttaaatttaaaaaaataaaaacaaaaaagtttgattgagacaatgaagag

SEQ ID NO: 8 - DNA, 3550 bp, Homo sapiens
aggctgcagaggtgccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttgatgagaactcagctgtccagctgcaaaggaaaagccaagtgagacgggctctgggaccatggtgaccaggctcttcccctgctccctggccctcgccagctgccaggctgaaaagaagcctcagctcccacaccgccctcctcaccgcccttcctcggcagtcacttccactggtggaccacgggcccccagccctgtgtcggccttgtctgtctcagctcaaccacagtctgacaccagagcccacttccatcctctctggtgtgaggcacagcgagggcagcatctggaggagctctgcagcctccacacctaccacgacctcccagggctgggctcaggaaaaaccagccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagcttttgattgaaagtcctagggtgattctatttctgctgtgatttatctgctgaaagctcagctggggttgtgcaagctagggacccattcctgtgtaatacaatgtctgcaccaatgctaataaagtcctattctcttttatgagaaagaaaaagacaccgtcctttaaagtgctgcagtatggccagacgtggtggctcacacctgcaatcccagcaccttaggaggccgaggcaggaggatccttgaggtcaggagttcgagaccagcctcgccaacatggtgaaaccccatttctactaaaaatacaaaaaattagccaagtgtggtggcatatgcctgtaatcccaactactcagaaggccgaggcaggagaattacttgaacgcaggagaatcactgcagcccaggaggcagaggttgcagtgagccgagattgcaccactgcactccagcctgggtgacagagcaagactccatctcagtaaataaataaataaataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccactgtcaggcctctgagcccaagctaagccatcatatcccctgtgccctgcacgtatacacccagatggcctgaagcaactgaagatccacaaaagaagtgaaaatagccagttcctgccttaactgatgacattccaccattgtgatttgttcctgccccaccctaactgatcaattgaccttgtgacaatacaccttccccacccttgagaaggtgctttgtaatattctccccacccaccccacgcccgcacccccgcacccttaagaaggtattttgtaatattctctccgccattgagaatgtgctttgtaagatccaccccctgcccacaaaaaattgctcctaactccaccgcctatcccaaacctacaagaactaatgataatcccaccaccctttgctgactctttttggactcagcccacctgcacccaggtgattaaaaagctttattgttcacacaaagcctgtttggtagtctcttcacagggaagcatgtgacacccacaatcccacctagcccaggagagagctacggcagggtgtgtgttttgacactgagcttggggctttttccatcttctccccacagcctctggctccacacctccaccgttcaagcgccagaaagagctgtctatgcagcctgctcttgggcctggggatgagacacacaattcattggctcctggattttaagtagacatttgtaaatctatagctaactactgtccttaaagccattgtttccattacaaaatccaactctctgagagaaaagggtgttttaaatttaaaaaaataaaaacaaaaaagtttgattgagacaa

SEQ ID NO: 9 - DNA, 2850 bp, Homo sapiens
tgtaaatgctcttctgactaatgcaaaccatgtgtccatagaaccagaagatttttccaggggaaaagagcccccacgccccgcccagctataaggggccatgcaccaagcagggtacccaggctgcagaggtgccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttgatgagaactcagctgtccagaaaaagacaccgtcctttaaagtgctgcagtatggccagacgtggtggctcacacctgcaatcccagcaccttaggaggccgaggcaggaggatccttgaggtcaggagttcgagaccagcctcgccaacatggtgaaaccccatttctactaaaaatacaaaaaattagccaagtgtggtggcatatgcctgtaatcccaactactcagaaggccgaggcaggagaattacttgaacgcaggagaatcactgcagcccaggaggcagaggttgcagtgagccgagattgcaccactgcactccagcctgggtgacagagcaagactccatctcagtaaataaataaataaataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccactgtcaggcctctgagcccaagctaagccatcatatcccctgtgccctgcacgtatacacccagatggcctgaagcaactgaagatccacaaaagaagtgaaaatagccagttcctgccttaactgatgacattccaccattgtgatttgttcctgccccaccctaactgatcaattgaccttgtgacaatacaccttccccacccttgagaaggtgctttgtaatattctccccacccaccccacgcccgcacccccgcacccttaagaaggtattttgtaatattctctccgccattgagaatgtgctttgtaagatccaccccctgcccacaaaaaattgctcctaactccaccgcctatcccaaacctacaagaactaatgataatcccaccaccctttgctgactctttttggactcagcccacctgcacccaggtgattaaaaagctttattgttcacacaaagcctgtttggtagtctcttcacagggaagcatgtgacacccacaatcccacctagcccaggagagagctacggcagggtgtgtgttttgacactgagcttggggctttttccatcttctccccacagcctctggctccacacctccaccgttcaagcgccagaaagagctgtctatgcagcctgctcttgggcctggggatgagacacacaattcattggctcctggattttaagtagacatttgtaaatctatagctaactactgtccttaaagccattgtttccattacaaaatccaactctctgagagaaaagggtgttttaaatttaaaaaaataaaaacaaaaaagtttgattgagacaatgaagag

SEQ ID NO: 10 - DNA, 2567 bp, Homo sapiens
aggctgcagaggtgccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagaaaaagacaccgtcctttaaagtgctgcagtatggccagacgtggtggctcacacctgcaatcccagcaccttaggaggccgaggcaggaggatccttgaggtcaggagttcgagaccagcctcgccaacatggtgaaaccccatttctactaaaaatacaaaaaattagccaagtgtggtggcatatgcctgtaatcccaactactcagaaggccgaggcaggagaattacttgaacgcaggagaatcactgcagcccaggaggcagaggttgcagtgagccgagattgcaccactgcactccagcctgggtgacagagcaagactccatctcagtaaataaataaataaataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccactgtcaggcctctgagcccaagctaagccatcatatcccctgtgccctgcacgtatacacccagatggcctgaagcaactgaagatccacaaaagaagtgaaaatagccagttcctgccttaactgatgacattccaccattgtgatttgttcctgccccaccctaactgatcaattgaccttgtgacaatacaccttccccacccttgagaaggtgctttgtaatattctccccacccaccccacgcccgcacccccgcacccttaagaaggtattttgtaatattctctccgccattgagaatgtgctttgtaagatccaccccctgcccacaaaaaattgctcctaactccaccgcctatcccaaacctacaagaactaatgataatcccaccaccctttgctgactctttttggactcagcccacctgcacccaggtgattaaaaagctttattgttcacacaaagcctgtttggtagtctcttcacagggaagcatgtgacacccacaatcccacctagcccaggagagagctacggcagggtgtgtgttttgacactgagcttggggctttttccatcttctccccacagcctctggctccacacctccaccgttcaagcgccagaaagagctgtctatgcagcctgctcttgggcctggggatgagacacacaattcattggctcctggattttaagtagacatttgtaaatctatagctaactactgtccttaaagccattgtttccattacaaaatccaactctctgagagaaaagggtgttttaaatttaaaaaaataaaaacaaaaaagtttgattgagacaatgaagag

SEQ ID NO: 11 - protein, 358 aa, Homo sapiens
MAESHLLQWLLLLLPTLCGPGTAAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFQNQTDSNGICMHLGLCKSRQPEPEQEPGMSDPLPKPLRDPLPDPLLDKLVLPVLPGALQARPGPHTQDLSEQQFPIPLPYCWLCRALIKRIQAMIPKGALAVAVAQVCRVVPLVAGGICQCLAERYSVILLDTLLGRMLPQLVCRLVLRCSMDDSAGPRSPTGEWLPRDSECHLCMSVTTQAGNSSEQAIPQAMLQACVGSWLDREKKKTPSFKVLQYGQTWWLTPAIPAP

SEQ ID NO: 12 - protein, 381 aa, Homo sapiens; identical to SEQ ID NO: 1

SEQ ID NO: 13 - protein, 197 aa, Homo sapiens [sequence omitted]

SEQ ID NO: 14 - protein, 191 aa, Homo sapiens
MDVGSKEVLMESPPDYSAAPRGRFGIPCCPVHLKRLLIVVVVVVLIVVVIVGALLMGLHMSQKHTEMVLEMSIGAPEAQQRLALSEHLVTTATFSIGSTGLVVYDYQQLLIAYKPAPGTCCYIMKIAPESIPSLEALTRKVHNFQAKPAVPTSKLGQAEGRDAGSAPSGGDPAFLGMAVSTLCGEVPLYYI

SEQ ID NO: 15 - protein, 521 aa, Homo sapiens
MLCALLLLPSLLGATRASPTSGPQECAKGSTVWCQDLQTAARCGAVGYCQGAVWNKPTAKSLPCDVCQDIAAAAGNGLNPDATESDILALVMKTCEWLPSQESSAGCKWMVDAHSSAILSMLRGAPDSAPAQVCTALSLCEPLQRHLATLRPLSKEDTFEAVAPFMANGPLTFHPRQAPEGALCQDCVRQVSRLQEAVRSNLTLADLNIQEQCESLGPGLAVLCKNYLFQFFVPADQALRLLPPQELCRKGGFCEELGAPARLTQVVAMDGVPSLELGLPRKQSEMQMKAGVTCEVCMNVVQKLDHWLMSNSSELMITHALERVCSVMPASITKECIILVDTYSPSLVQLVAKITPEKVCKFIRLCGNRRRARAVHDAYAIVPSPEWDAENQGSFCNGCKRLLTVSSHNLESKSTKRDILVAFKGGCSILPLPYMIQCKHFVTQYEPVLIESLKDMMDPVAVCKKVGACHGPRTPLLGTDQCALGPSFWCRSQEAAKLCNAVQHCQKHVWKEMHLHAGEHA

SEQ ID NO: 16 - protein, 504 aa, Artificial Sequence
  Description of Artificial Sequence Synthetic polypeptide
MHHHHHHAGMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSPEFAWTTSSLACAQGPEFWCQSLEQALQCRALGHCLQEVWGHVGADDLCQECEDIVHILNKMAKEAIFQDTMRKFLEQECNVLPLKLLMPQCNQVLDDYFPLVIDYFNQTDSNGICMHLGLCKSRQPEPEQEPGMSDPLPKPLRDPLPDPLLDKLVLPVLPGALQARPGPHTQDLSEQQFLVPRGSPIPLPYCWLCRALIKRIQAMIPKGALAVAVAQVCRVVPLVAGGICQCLAERYSVILLDTLLGRMLPQLVCRLVLRCSM

SEQ ID NO: 17 - protein, 6 aa, Artificial Sequence
  Description of Artificial Sequence Synthetic 6xHis tag
HHHHHH

SEQ ID NO: 18 - protein, 8 aa, Artificial Sequence
  Description of Artificial Sequence Synthetic peptide
DYKDDDDK

SEQ ID NO: 19 - DNA, 65 bp, Artificial Sequence
  Description of Artificial Sequence Synthetic oligonucleotide
gaagcaatcctgttccatgccctagaaccccaaagccatctgcaattgtgggcctgtgcccacgt

SEQ ID NO: 20 - protein, 6 aa, Artificial Sequence
  Description of Artificial Sequence Synthetic peptide
LVPRGS

SEQ ID NO: 21 - protein, 79 aa, Artificial Sequence
  Description of Artificial Sequence Synthetic polypeptide
  MOD_RES (5)..(6): Any amino acid
  MOD_RES (8)..(8): Any amino acid
  MOD_RES (13)..(15): Any amino acid
  MOD_RES (18)..(18): Any amino acid
  MOD_RES (26)..(26): Any amino acid
  MOD_RES (33)..(33): Any amino acid
  MOD_RES (37)..(37): Any amino acid
  MOD_RES (40)..(42): Any amino acid
  MOD_RES (45)..(45): Any amino acid
  MOD_RES (49)..(49): Any amino acid
  MOD_RES (56)..(56): Any amino acid
  MOD_RES (63)..(63): Any amino acid
  MOD_RES (68)..(69): Any amino acid
  MOD_RES (73)..(73): Any amino acid
DDYCXXCXDIVTXXXKMXKKNSTEQXIIVAIEXECSXLPXXXSEXCKQXVDQYSPXILDIIIXEMDPXXVCSXLGICSS

SEQ ID NO: 22 - protein, 76 aa, Homo sapiens
DSECHLCMSVTTQAGNSSEQAIPQAMLQACVGSWLDREKCKQFVEQHTPQLLTLVPRGWDAHTTCQALGVCGTMSS

SEQ ID NO: 23 - protein, 80 aa, Homo sapiens
LPCDICKDVVTAAGDMLKDNATEEEILVYLEKTCDWLPKPNMSASCKEIVDSYLPVILDIIKGEMSRPGEVCSALNLCES

SEQ ID NO: 24 - protein, 78 aa, Homo sapiens
DVCQDCIQMVTDIQTAVRTNSTFVQALVEHVKEECDRLGPGMADICKNYISQYSEIAIQMMMHMQPKEICALVGFCDE

SEQ ID NO: 25 - protein, 79 aa, Homo sapiens
DVYCEVCEFLVKEVTKLIDNNKTEKEILDAFDKMCSKLPKSLSEECQEVVDTYGSSILSILLEEVSPELVCSMLHLCSG

SEQ ID NO: 26 - protein, 80 aa, Homo sapiens
DGGFCEVCKKLVGYLDRNLEKNSTKQEILAALEKGCSFLPDPYQKQCDQFVAEYEPVLIEILVEVMDPSFVCLKIGACPS

SEQ ID NO: 27 - protein, 78 aa, Homo sapiens
PIPLPYCWLCRALIKRIQAMIPKGALAVAVAQVCRVVPLVAGGICQCLAERYSVILLDTLLGRMLPQLVCRLVLRCSM